(19) United States
(12) Patent Application Publication
(10) Pub. No.: US 2006/0013791 A1
(43) Pub. Date: Jan. 19, 2006

(54) Cosmetic composition comprising a defined silicone polymer and a film former

(75) Inventors: Momoko Shimizu, Kawasaki-shi (JP); Xavier Blin, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/181,976

(22) Filed: Jul. 15, 2005

Related U.S. Application Data
(60) Provisional application No. 60/591,927, filed on Jul. 29, 2004.

(30) Foreign Application Priority Data
Jul. 16, 2004 (FR) 04 51559

(51) Int. Cl. A61K 8/894

(52) U.S. Cl. 424/70.12

(57) ABSTRACT

Cosmetic makeup and/or care compositions for application to the skin, lips and/or epidermal derivatives may exhibit improved staying power, satisfactory or improved gloss and improved comfort. The cosmetic compositions may include a film former and a silicon polymer.

### CROSS-REFERENCE TO RELATED APPLICATIONS

This non-provisional application claims the benefit of French Application No. 04 51559 filed on Jul. 16, 2004 and U.S. Provisional Application No. 60/591,927 filed on Jul. 29, 2004.

### BACKGROUND

The present invention includes cosmetic makeup and/or care compositions for application to the skin, lips and/or epidermal derivatives, comprising a particular silicone polymer and a film former.

The disclosed compositions provide improved staying power, satisfactory or even improved gloss and improved comfort.

The cosmetic compositions are, more particularly, makeup and/or care products for application to the skin, lips and/or epidermal derivatives, especially lipsticks; lip balms; lip pencils; liquid foundations or solid foundations, cast in particular as a stick or in a dish; concealer products and skin colouring products; temporary tattoos; eye makeup products, such as eyeliners, in particular in the form of pencils, mascaras, in particular in the form of cakes, and eye shadows.

Generally speaking, the purpose of cosmetic compositions is to impart an aesthetic effect at the time of application and to maintain this aesthetic effect over time. Cosmetic compositions are required, in particular, to withstand the various external factors that are liable to alter their aesthetic effect, such as perspiration or tears, in the case of a foundation, or saliva, in the case of a lipstick.

Cosmetic compositions, such as lipsticks, for example, must not migrate into fine lines or wrinkles, or undergo transfer to a fabric. Cosmetic compositions must also be pleasant to apply and must maintain a sensation of comfort over time, while retaining satisfactory aesthetic properties.

Japanese patent applications JP 6-305933 and JP 7-330547 describe compositions comprising silicone alkylglyceryl ether derivatives. Compositions having an oily base and comprising polyglycerylated silicone derivatives or fluoroalkylpolyglycerylated silicone derivatives have been proposed in JP 6-157236, JP 9-071504 and JP 10-310504. Compositions comprising silicone alkylglycerol derivatives have also been described in EP 0 475 130 and in JP 2-844453 and JP 2-587797. Other compositions comprising silicone derivatives hydroxylated with saccharides, with butylene glycol or with glycerol have been described in JP 5-186596 and JP 6-145023.

### SUMMARY

However, there presently is no completely satisfactory cosmetic composition that has desired aesthetic characteristics and that can provide satisfactory staying power, gloss and comfort.

Exemplary embodiments of the present invention provide anhydrous cosmetic makeup and/or care compositions comprising film formers and silicon polymers that exhibit improved staying power, satisfactory or improved gloss characteristics and improved comfort when applied to the skin, lips and/or epidermal derivatives.

Exemplary anhydrous cosmetic compositions according to the present invention include, in a physiologically acceptable medium, at least one film former and at least one silicone polymer of general formula (I), R¹ₐR²_{b}R³_{c}SiO_{(4-a-b-c)/2}. In general formula (I), a ranges from 1 to 2.5, b and c independently range from 0.001 to 1.5, R¹, which may be identical or different at each occurrence, is a radical chosen from the group consisting of C₁ to C₃₀ alkyl radicals, C₁ to C₃₀ alkyl radicals substituted with one or more fluorine atoms, amino and/or carboxyl groups, aryl and aralkyl radicals, radicals of general formula (II) —C_{d}H_{2d}—O—(C₂H₄O)ₑ(C₃H₆O)_{f}R⁴, R² is a radical represented by the general formula (III) -Q-O—X, and R³ is an organosiloxane group of general formula (IV) 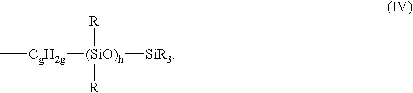
In general formula (II), R⁴ is a C₁ to C₃₀ hydrocarbon radical or a R⁵—(CO)—, in which R⁵ is a C₁ to C₃₀ hydrocarbon radical, d is an integer in a range of from 0 to 15, and e and f are each independently integers in a range of from 0 to 50, and combinations thereof. In general formula (III), Q is a divalent C₂ to C₂₀ hydrocarbon radical that can include at least one ether bond and/or at least one ester bond, and X is a polyhydroxylated hydrocarbon radical. In addition, when R² is a radical of general formula (IIIA): —C₃H₆—O[CH₂CH(OH)CH₂O]ₙH, and n is an integer in a range of from 1 to 5, R¹ is not a C₁₂ alkyl radical. In general formula (IV), each R is an independent radical chosen from C₁ to C₃₀ alkyl radicals, C₁ to C₃₀ alkyl radicals substituted with one or more fluorine atoms, and aryl and aralkyl radicals, and g is an integer ranging from 1 to 5, and h is an integer ranging from 0 to 500.

Exemplary anhydrous cosmetic compositions according to the present invention include, in a physiologically acceptable medium, at least one film former and at least one silicone polymer of general formula (I), R¹ₐR²_{b}R³_{c}SiO_{(4-a-b-c)/2}. In general formula (I), a ranges from 1 to 2.5, b and c independently range from 0.001 to 1.5, R¹, which may be identical or different at each occurrence, is a radical chosen from the group consisting of C₁ to C₃₀ alkyl radicals, C₁ to C₃₀ alkyl radicals substituted with one or more fluorine atoms, amino and/or carboxyl groups, aryl and aralkyl radicals, radicals of general formula (II) —C_{d}H_{2d}—O—(C₂H₄O)ₑ(C₃H₆O)_{f}R⁴, R² is a radical represented by the general formula (III) -Q-O—X, and R³ is an organosiloxane group of general formula (IV) 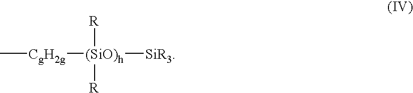
In general formula (II), R⁴ is a C₁ to C₃₀ hydrocarbon radical or a R⁵—(CO)—, in which R⁵ is a C₁ to C₃₀ hydrocarbon radical, d is an integer in a range of from 0 to 15, and e and f are each independently integers in a range of from 0 to 50, and combinations thereof. In general formula (III), Q is a divalent C₂ to C₂₀ hydrocarbon radical that can include at least one ether bond and/or at least one ester bond, and X is a polyhydroxylated hydrocarbon radical. In general formula (IV), each R is an independent radical chosen from C₁ to C₃₀ alkyl radicals, C₁ to C₃₀ alkyl radicals substituted with one or more fluorine atoms, and aryl and aralkyl radicals, and g is an integer ranging from 1 to 5, and h is an integer ranging from 0 to 500. The film former of such exemplary embodiments is present in a weight ratio, relative to the weight of the silicone polymer of general formula (I), of less than 5:1.

Exemplary anhydrous cosmetic compositions according to the present invention include, in a physiologically acceptable medium, at least one non-crystalline film former, at least one silicone compound different from the film former, and at least one silicone polymer of general formula (I), R¹ₐR²_{b}R³_{c}SiO_{(4-a-b-c)/2}. In general formula (I), a ranges from 1 to 2.5, b and c independently range from 0.001 to 1.5, R¹, which may be identical or different at each occurrence, is a radical chosen from the group consisting of C₁ to C₃₀ alkyl radicals, C₁ to C₃₀ alkyl radicals substituted with one or more fluorine atoms, amino and/or carboxyl groups, aryl and aralkyl radicals, radicals of general formula (II) —C_{d}H_{2d}—O—(C₂H₄O)ₑ(C₃H₆O)_{f}R⁴, R² is a radical represented by the general formula (III) -Q-O—X, and R³ is an organosiloxane group of general formula (IV) 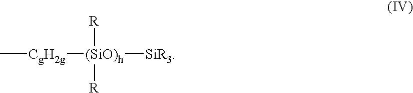
In general formula (II), R⁴ is a C₁ to C₃₀ hydrocarbon radical or a R⁵—(CO)—, in which R⁵ is a C₁ to C₃₀ hydrocarbon radical, d is an integer in a range of from 0 to 15, and e and f are each independently integers in a range of from 0 to 50, and combinations thereof. In general formula (III), Q is a divalent C₂ to C₂₀ hydrocarbon radical that can include at least one ether bond and/or at least one ester bond, and X is a polyhydroxylated hydrocarbon radical. In general formula (IV), each R is an independent radical chosen from C₁ to C₃₀ alkyl radicals, C₁ to C₃₀ alkyl radicals substituted with one or more fluorine atoms, and aryl and aralkyl radicals, and g is an integer ranging from 1 to 5, and h is an integer ranging from 0 to 500.

Exemplary lip makeup cosmetic compositions according to the present invention include, in a physiologically acceptable medium, at least one film former and at least one silicone polymer of general formula (I), R¹ₐR²_{b}R³_{c}SiO_{(4-a-b-c)/2}. In general formula (I), a ranges from 1 to 2.5, b and c independently range from 0.001 to 1.5, R¹, which may be identical or different at each occurrence, is a radical chosen from the group consisting of C₁ to C₃₀ alkyl radicals, C₁ to C₃₀ alkyl radicals substituted with one or more fluorine atoms, amino and/or carboxyl groups, aryl and aralkyl radicals, radicals of general formula (II) —C_{d}H_{2d}—O—(C₂H₄O)ₑ(C₃H₆O)_{f}R⁴, R² is a radical represented by the general formula (III) -Q-O—X, and R³ is an organosiloxane group of general formula (IV) 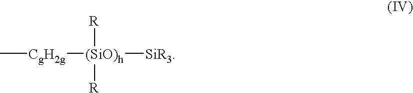
In general formula (II), R⁴ is a C₁ to C₃₀ hydrocarbon radical or a R⁵—(CO)—, in which R⁵ is a C₁ to C₃₀ hydrocarbon radical, d is an integer in a range of from 0 to 15, and e and f are each independently integers in a range of from 0 to 50, and combinations thereof. In general formula (III), Q is a divalent C₂ to C₂₀ hydrocarbon radical that can include at least one ether bond and/or at least one ester bond, and X is a polyhydroxylated hydrocarbon radical. In general formula (IV), each R is an independent radical chosen from C₁ to C₃₀ alkyl radicals, C₁ to C₃₀ alkyl radicals substituted with one or more fluorine atoms, and aryl and aralkyl radicals, and g is an integer ranging from 1 to 5, and h is an integer ranging from 0 to 500.

Exemplary cosmetic compositions according to the present invention include, in a physiologically acceptable medium, at least one hydrocarbon ester containing less than 40 carbon atoms, at least one film former chosen from non-silicone film formers and silicone film formers devoid of acrylic units, and at least one silicone polymer of general formula (I), R¹ₐR²_{b}R³_{c}SiO_{(4-a-b-c)/2}. In general formula (I), a ranges from 1 to 2.5, b and c independently range from 0.001 to 1.5, R¹, which may be identical or different at each occurrence, is a radical chosen from the group consisting of C₁ to C₃₀ alkyl radicals, C₁ to C₃₀ alkyl radicals substituted with one or more fluorine atoms, amino and/or carboxyl groups, aryl and aralkyl radicals, radicals of general formula (II) —C_{d}H_{2d}—O—(C₂H₄O)ₑ(C₃H₆O)_{f}R⁴, R² is a radical represented by the general formula (III)-Q-O—X, and R³ is an organosiloxane group of general formula (IV) 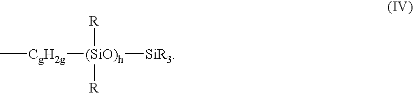
In general formula (II), R⁴ is a C₁ to C₃₀ hydrocarbon radical or a R⁵—(CO)—, in which R⁵ is a C₁ to C₃₀ hydrocarbon radical, d is an integer in a range of from 0 to 15, and e and f are each independently integers in a range of from 0 to 50, and combinations thereof. In general formula (III), Q is a divalent C₂ to C₂₀ hydrocarbon radical that can include at least one ether bond and/or at least one ester bond, and X is a polyhydroxylated hydrocarbon radical. In general formula (IV), each R is an independent radical chosen from C₁ to C₃₀ alkyl radicals, C₁ to C₃₀ alkyl radicals substituted with one or more fluorine atoms, and aryl and aralkyl radicals, and g is an integer ranging from 1 to 5, and h is an integer ranging from 0 to 500.

### DETAILED DESCRIPTION OF EMBODIMENTS

Exemplary embodiments of cosmetic compositions include polyglycerylated silicone polymers and exhibit improved staying power on keratin materials, without detrimental effects on gloss characteristics. In embodiments, exemplary cosmetic compositions exhibit improved gloss characteristics and/or improved comfort.

As used herein, the term “keratin materials” is includes, but is not limited to, the skin, mucosae, such as the lips, the nails and the keratin fibres, as exemplified by the eyelashes and the hair.

Exemplary cosmetic compositions may be applied on the skin and lips.

Use of a silicone polymer of general formula (I):
R¹ₐR²_{b}R³_{c}SiO_{(4-a-b-c)/2} (I)
in which R¹ is an alkyl radical, R² is represented by the general formula (III):
   Q-O—X (III)
in which Q is a divalent hydrocarbon radical and X is a polyhydroxylated hydrocarbon radical, and R³ is an organosiloxane group of general formula (IV): 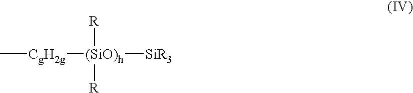
in which the radicals R are each independently an alkyl radical, in combination with at least one film former provides cosmetic compositions that have improved staying power without detriment to the gloss. In some embodiments, such cosmetic compositions can exhibit improved gloss and/or comfort.

In exemplary embodiments, anhydrous cosmetic compositions including, in a physiologically acceptable medium, at least one film former present in a weight ratio, relative to the weight of the silicone polymer of general formula (I), of less than 5:1, in particular less than 1:1, and more particularly less than or equal to 1:2, and at least one silicone polymer of general formula (I):
R¹ₐR²_{b}R³_{c}SiO_{(4a-b-c)/2} (I)
in which:
a is an integer in the range of from 1 to 2.5, and b and c are each independently in the range of from 0.001 to 1.5.

In general formula (I), R¹, which may be the same or different in each occurrence of R¹, is chosen from substituted and unsubstituted C₁ to C₃₀ alkyl radicals in which the substituents may be one or more fluorine atoms, amino and/or carboxyl groups, aryl and aralkyl radicals, and radicals of general formula (II):
—C_{d}H_{2d}—O—(C₂H₄O)ₑ(C₃H₆O)_{f}R⁴ (II).

In general formula (II), R⁴ is a radical that is chosen from C₁ to C₃₀ hydrocarbon radicals and R⁵—(CO)— radicals in which R⁵ is a C₁ to C₃₀ hydrocarbon radical, andd is an integer in the range of from 0 to 15 and e and f are independently chosen integers in the range of from 0 to 50, and combinations thereof,

In general formula (I), R² is a radical represented by the general formula (III):
Q-O—X (III)
in which Q is a divalent C₂ to C₂₀ hydrocarbon radical that may include at least one ether bond and/or at least one ester bond, and X is a polyhydroxylated hydrocarbon radical.

R³, in general formula (I), is an organosiloxane of general formula (IV): 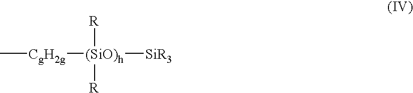
in which each R is independently chosen from substituted and unsubstituted C₁ to C₃₀ alkyl radicals, in which the substituents may be one or more fluorine atoms, and aryl and aralkyl radicals, g is an integer in the range of from 1 to 5 and h is an integer in the range of from 0 to 500.

In additional exemplary embodiments, anhydrous cosmetic compositions include, in a physiologically acceptable medium, at least one non-crystalline film former, a silicone compound other than the film former, and at least one silicone polymer of general formula (I), as defined above.

Additional exemplary embodiments provide cosmetic lip makeup compositions that include, in a physiologically acceptable medium, at least one film former selected from non-silicone film formers and silicone film formers devoid of an acrylic unit, and at least one silicone polymer of general formula (I), as defined above.

Additional exemplary embodiments provide cosmetic compositions that include, in a physiologically acceptable medium, at least one hydrocarbon ester containing less than 40 carbon atoms, at least one film former selected from non-silicone film formers and silicone film formers devoid of an acrylic unit, and at least one silicone polymer of general formula (I), as defined above.

In additional exemplary embodiments, anhydrous cosmetic compositions include, in a physiologically acceptable medium, at least one film former and at least one silicone polymer of general formula (I), as defined above, with the proviso that, when R² is a group of general formula (IIIA):
—C₃H₆—O[CH₂CH(OH)CH₂O]ₙH (IIIA)
in which n is an integer in the range of from 1 to 5, R¹ is not a C₁₂ alkyl radical.

Further exemplary embodiments include cosmetic compositions for making up and/or caring for the lips and/or the skin, and in particular provide a lipstick.

Exemplary embodiments may also provide a synthetic support on which is present, over some or all of its surface, at least one layer of a cosmetic composition according to the invention.

Further exemplary embodiments provide methods of making up and/or caring for keratin materials, such as the skin and/or lips, which include applying to the keratin materials at least one cosmetic composition in accordance with the present invention.

Exemplary embodiments also provide for the use of at least one silicone polymer of general formula (I), as defined above, in combination with at least one film former to prepare a cosmetic composition exhibiting improved staying power, gloss and comfort.

As used herein, “improved staying power” encompasses improved water resistance and/or oil resistance and/or reduced transfer and/or migration.

The cosmetic compositions according to exemplary embodiments of the invention may be in the form of a paste, liquid, gel, cream or solid. In particular embodiments, cosmetic compositions are in cast form, such as in the form of a stick. Cosmetic compositions of exemplary embodiments may also be in the form of simple oil-in-water or water-in-oil emulsions or multiple emulsions, or in the form of anhydrous, solid or flexible gels.

In particular embodiments, cosmetic compositions are in an anhydrous form.

The term “composition in cast form,” as used herein, encompasses solid or semi-solid compositions obtained after cooling compositions that have been introduced into a mold while in a liquid or melt state. These exemplary cosmetic compositions may be cast in the form of a stick or crayon, or in a dish.

In particular exemplary embodiments, cosmetic compositions according to the invention are in cast form, i.e., in solid or semi-solid form, and may be in the form of a stick.

To determine the hardness of a cosmetic composition in accordance with exemplary embodiments, a stick of the composition having a circular section 12.7 mm in diameter may be prepared. The stick may be cast and then kept at a temperature of 20° C. for 24 hours before measurement.

The hardness may be measured by the “cheese wire” method, which consists of cutting the stick transversely by means of a rigid tungsten wire 250 μm in diameter, by advancing the wire relative to the stick at a speed of 100 mm/min. The hardness corresponds to the maximum shearing force exerted by the wire on the stick at 20° C., this force being measured by means of a DFGS2 dynamometer sold by Indelco-Chatillon. The hardness is expressed in grams.

According to this method, the hardness of cosmetic compositions of exemplary embodiments in accordance with the invention that are in stick form may vary from about 50 to about 300 g, such as from about 70 to about 250 g or from about 100 to about 230 g.

Exemplary embodiments of cosmetic compositions according to the present invention may exhibit improved colour permanence, as manifested, for example, by reduced migration or transfer of colour, improved colour fastness to water improved colour fastness to oil, and/or reduced migration during application of the cosmetic compositions.

Exemplary embodiments of cosmetic compositions according to the present invention may also maintain a comfortable sensation and can lack a sticky sensation while exhibiting effective adhesion to the skin.

Further, exemplary embodiments of cosmetic compositions according to the present invention may maintain the aesthetic effect, particularly the gloss effect, over time.

Exemplary embodiments of cosmetic compositions according to the present invention may be able to impart a soft and smooth sensation and to maintain effective moisturizing.

In exemplary embodiments, cosmetic compositions according to the present invention exhibit good staying power with regard to external factors that may modify aesthetic properties, such as perspiration or consumption of a meal, in the case of a lipstick.

Silicone Polymer of General Formula (I)

The silicone polymers in accordance with the silicone polymer of general formula (I) and possible for use in exemplary embodiments of cosmetic compositions according to the present invention are described in detail in EP 1 213 316, which is incorporated by reference in its entirety herein.

The silicone polymers of general formula (I) may be used, in embodiments, as a surfactant and/or as an oily base.

Silicone polymers of general formula (I) that suitable for use in embodiments of the cosmetic compositions in accordance with the invention do not possess surface treatment functionality.

Introduced in sufficient quantity in embodiments, silicone polymers of general formula (I) may improve staying power, gloss and/or comfort of exemplary cosmetic compositions according to the invention.

In particular embodiments, the silicone polymers are represented by the general formula (I) below:
R¹ₐR²_{b}R³_{c}SiO_{(4-a-b-c)/2} (I)
in which:
a) a ranges from 1 to 2.5; and b and c independently of one another range from 0.001 to 1.5;
b) R¹, which may be the same or different for each occurrence, is selected from:
   substituted and unsubstituted C₁ to C₃₀ alkyl radicals in which one or more fluorine atoms and/or amino and/or carboxyl groups may be substituents,
   aryl and aralkyl radicals,
   radicals of general formula (II):
      —C_{d}H_{2d}—O—(C₂H₄O)ₑ(C₃H₆O)_{f}R⁴ (II)
in which:
   R⁴ is a C₁ to C₃₀ hydrocarbon radical or a radical R⁵—(CO)—, and R⁵ is a C₁ to C₃₀ hydrocarbon radical, and
   d is an integer in the range of from 0 to 15 and e and f, independently of one another, are each integers in the range of from 0 to 50, and
   combinations thereof.
c) R² is represented by the general formula (III) below:
   Q-O—X (III)
in which:
   Q is a divalent C₂ to C₂₀ hydrocarbon radical that can include at least one ether bond and/or at least one ester bond, and
   X is a polyhydroxylated hydrocarbon radical.
d) R³ is an organosiloxane group of general formula (IV): 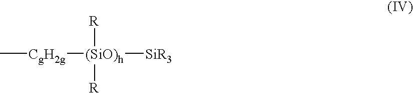
in which each R is independently selected from substituted and unsubstituted C₁ to C₃₀ alkyl radicals in which one or more fluorine atoms may be substituents, and aryl and aralkyl radicals,
   g is an integer in the range of from 1 to 5 and h is an integer in the range of from 0 to 500.

When the radicals R are radicals selected from substituted and unsubstituted C₁ to C₃₀ alkyl radicals in which one or more fluorine atoms may be substituents, and from aryl radicals and aralkyl radicals, R has the same meaning as the radical R¹ as defined above.

It should be noted that the radicals R¹, R² and R³ of the silicone polymers of general formula (I), as defined above, are distributed randomly or statistically; that is, R¹, R² and R³ appear in the structure of the polymer without a determined order. Similarly, R¹, R² and R³ may respectively feature radicals of different kind in a compound of general formula

In a particular exemplary embodiment,
in a):
   a is in the range of from 1.2 to 2.3, and b and c, independently of one another, are each in the range of from 0.05 to 1.
In b):
   when R¹ is an alkyl radical, R¹ may be a C₁ to C₃₀ alkyl radical, for example, a C₁ to C₂₅ alkyl radical, such as a C₁ to C₂₀ alkyl radical, a C₁ to C₁₀ alkyl radical, a C₁ to C₆ alkyl radical, or a C₁, to C₄ alkyl radical. In some embodiments, R¹ may be a methyl, ethyl, n- or isopropyl, n- or iso- or tert-butyl, pentyl, hexyl, heptyl, octyl, nonyl, decyl, undecyl or lauryl radical. R¹ may also be a cycloalkyl radical, such as a cyclopropyl, a cyclobutyl, a cyclopentyl or a cyclohexyl. R¹ may also be a linear or branched, monounsaturated or polyunsaturated alkyl radical. R¹ may also be an alkyl radical substituted by one or more fluorine atoms, such as trifluoropropyl or heptadecafluorodecyl. In embodiments, R¹ may also be an alkyl radical substituted by one or more amino groups, such as 2-aminoethyl, 3-aminopropyl, and (2-ethylamino)-3-aminopropyl, or an alkyl group substituted by one or more carboxyl groups, such as 3-carboxypropyl.
   R¹ may also be an aryl or aralkyl radical, such as the phenyl radical, the tolyl radical, the benzyl radical and the phenethyl radical.
   R¹ may also be an organic group represented by the general formula
      —C_{d}H_{2d}—O—(C₂H₄O)ₑ(C₃H₆O)_{f}R⁴ (II)

According to some exemplary embodiments, R¹ may be a hydroxylated radical or a radical obtained from the addition reaction of a saturated or unsaturated, linear or branched alkenyl ether in which d=0 and which is therefore of formula:
—O—(C₂H₄O)ₑ(C₃H₆O)_{f}R⁴

In this case, when e and fare zero, R¹ is an alkoxy group having from 4 to 30 carbon atoms, for example, a C₄ to C₁₀ lower alkoxy radical, such as butoxy or pentoxy, or a C₁₁ to C₃₀ higher alkoxy radical, such as oleoxy, stearoxy, viz., for example, cetyl alcohol, oleyl alcohol and stearyl alcohol, or a radical obtained from an acid or a fatty acid, such as acetic acid, lactic acid, butyric acid, oleic acid, stearic acid and behenic acid.

When e and fare greater than 1, R¹ is a hydroxyl radical originating from the addition reaction of an alkylene oxide.

When e and f are zero, d may be, in some embodiments, 3, 5 or 11. In such embodiments, R¹ is an allyl ether, pentenyl ether or undecenyl ether radical or an allyl stearyl ether, pentenyl behenyl ether or undecenyl oleyl ether radical, depending on the nature of the substituent R⁴.

When e and/or fare not zero, an alkoxy radical and an ester radical are present via a polyoxyalkylene group in embodiments.

Regardless of the values of e and f, d may be, in embodiments, within the range of from 3 to 5.

According to an exemplary embodiment, the radical R¹ may be any one of the above-defined radicals or a combination of two or more of these radicals.

In exemplary embodiments, R¹ is an alkyl radical selected from the methyl radical, the lauryl radical, and combinations thereof.

In some exemplary embodiments, R¹ represents two or more radicals in a single general formula (I), a methyl radical and a lauryl radical, for example, these radicals appear in the structure at random and with a frequency that is specific to them.

In particular embodiments, methyl radicals are at least 50% of the radicals R¹, at least 70% of the radicals R¹, or 100% of the radicals R¹ are methyl radicals.
In c):
   Q may be a divalent hydrocarbon radical selected from:
   —(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₂—CH(CH₂CH₂CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂—.

In exemplary embodiments, Q may be a divalent radical selected from —(CH₂)₂— and —(CH₂)₃—.
X may be, in exemplary embodiments, a polyhydroxylated hydrocarbon radical containing at least two hydroxyl residues, such as a hydrocarbon group selected from glyceryl derivatives and saccharide derivatives.

The glycerol residues may be compounds having the following formulae, in which Q has the same meaning as in the general formula (III), and s and t are integers in the range of from 1 to 20, for example from 1 to 15, from 1 to 10, or from 1 to 5.

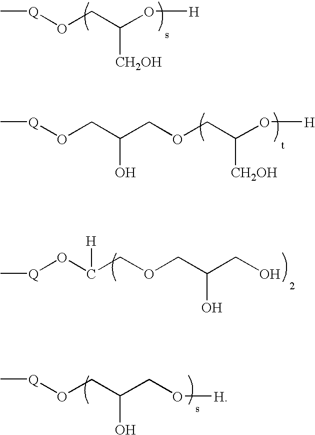

In the above formulae, one or more hydroxyl groups may be replaced by alkoxy groups or ester groups.

The saccharide radicals, which can be used in general formula (III) of embodiments, may be of monosaccharide type, such as glycosyl, mannosyl, galactosyl, ribosyl, arabinosyl, xylosyl or fructosyl groups, of oligosaccharide type, such as maltosyl, cellobiosyl, lactosyl or maltotriosyl, or a polysaccharide type, such as cellulose or starch.

In particular embodiments, the saccharide groups are of monosaccharide or oligosaccharide type.
In d):
   each of the radicals R may represent, independently of one another, a radical chosen from C₁ to C₂₀, such as C₁ to C₁₀, or C₁ to C₆ alkyl radicals, which may be substituted with one or more fluorine atoms. When the radicals R¹ represent a radical chosen from alkyl radicals as defined above, optionally substituted with one or more fluorine atoms, the radicals R have the same meaning as the radical R¹ as defined above.
   g, according to an exemplary embodiment, is 2.
   h, according to an exemplary embodiment, is within the range of from 1 to 50.

However, in certain exemplary embodiments, the silicone polymer of general formula (I) is such that, when the radical R² is represented by the general formula (IIIA):
—C₃H₆O[CH₂CH(OH)CH₂O]ₙH (IIIA)
in which n ranges from 1 to 5, the radical R¹ is not a C₁₂ alkyl radical.

According to an exemplary embodiment, the silicone polymer of general formula (I) is further defined as follows:
a ranges from 1 to 1.4 and b and c, independently of one another, range from 0.02 to 0.03, and
R¹ is a C₁ to C₁₀, such as C₁, to C₆, or C₁ to C₄ alkyl radical.
R² is represented by the formula (IIIA):
   —C₃H₆O[CH₂CH(OH)CH₂O]ₙH (IIIA)
in which:
n is in a range from 1 to 5, and
R³ is represented by the formula (IVA):
   —C₂H₄(CH₃)₂SiO[(CH₃)₂SiO]ₘ Si(CH₃)₃ (IVA)
in which:
m is in a range from 3 to 9.

According to additional exemplary embodiments, the silicone polymer of general formula (I), which can be used in the cosmetic compositions according to embodiments of the present invention, is such that:
a ranges from 1 to 1.4 and b and c, independently of one another, range from 0.02 to 0.04,
R¹ is a methyl radical,
R² is represented by the formula (IIIA) in which n ranges from 1 to 5, and
R³ is represented by the formula (IVA) in which m ranges from 3 to 9.

In exemplary embodiments, the silicone polymer of general formula (I) may be chosen from polyglyceryl-3 polymethylsiloxyethyl dimethicone, laurylpolyglyceryl-3 polymethylsiloxyethyl dimethicone and polyglyceryl-3 disiloxane dimethicone, whose formulae are, respectively:
polyglyceryl-3 polymethylsiloxyethyl dimethicone (formula (V)): 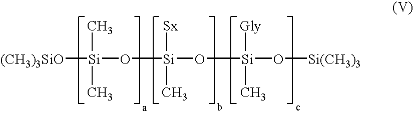
in which:
Sx: —C₂H₄[(CH₃)₂SiO]ₘSi(CH₃)₃
Gly: —C₃H₆O[CH₂—CH(OH)CH₂O]ₙH
with a=1-1.4, b=0.02-0.04, c=0.02-0.04, m=3-9, n=1-5,
lauryl polyglyceryl-3 polymethylsiloxyethyl dimethicone (formula (VI)): 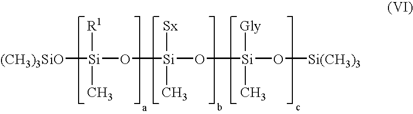
in which Sx, Gly, a, b, c, m and n have the same meaning as above and R¹ is either a methyl radical or a lauryl radical.
Polyglyceryl-3 disiloxane dimethicone (formula (VII)): 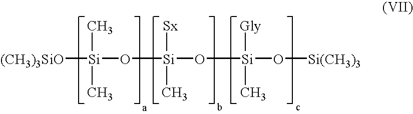
in which Gly, a, b, c, m and n have the same meaning as above, and
Sx: —O(CH₃)₂SiO—Si(CH₃)₃

The silicone polymer of general formula (I) may be present in the cosmetic compositions in accordance with exemplary embodiments of the present invention in a proportion of from about 0.1% to about 40% by weight, such as from 0. about 5% to about 30% by weight, from about 1% to about 25% by weight, from about 5% to about 20% by weight, or from about 7% to about 15% by weight, relative to the total weight of the composition.

The silicone polymer of general formula (I) may be employed in a free form in exemplary embodiments.

As used herein, the term “free form” encompasses forms of silicone polymers of general formula (I) in which the polymer is not employed in a form in which the polymer is combined with or adsorbed on another material, such as, for example, in EP 1 416 016 and EP 1 424 373, where the polymer is present in the form of a coating of a powder or a colorant for the purpose of blocking the surface activity of the particles making up the corresponding powder.

According to an exemplary embodiment, the silicone polymer of general formula (I) may be chosen from polymers sold by SHIN-ETSU under the references KF6100®, KF6104® and KF6105®.

The polymer sold under the reference KF6104® may be particularly suitable for the preparation of exemplary embodiments of the cosmetic compositions in accordance with the invention.

The KF6104®polymer, sold by SHIN-ETSU, may be particularly suitable for preparing exemplary embodiments of cosmetic compositions in accordance with the invention that exhibit improved staying power on keratin materials without detriment to the gloss or that may exhibit improved average gloss.

Physiologically Acceptable Medium

As used herein, a “physiologically acceptable medium” encompasses any non-toxic medium that can be applied to the skin, lips or keratin materials of human beings. The physiologically acceptable medium generally can be adapted to the nature of the substrate to which the composition is to be applied, and also to the form in which the composition is to be packaged.

The physiologically acceptable medium of embodiments may comprise an aqueous phase and/or a fatty phase.

In exemplary embodiments, the aqueous phase or the fatty phase may form the continuous phase of the composition.

Exemplary embodiments of cosmetic compositions in accordance with the present invention may be presented in the form of an emulsion, in which the silicone polymer of general formula (I), as defined above, may have the function of a surfactant.

As used herein, emulsions may contain a lipophilic phase and a hydrophilic phase, the latter not systematically being water.

Thus, the cosmetic compositions of exemplary embodiments in accordance with the invention may be in the form of a water-in-oil, oil-in-water, multiple or anhydrous emulsion.

In particular embodiments, the cosmetic compositions in accordance with the invention may be in the form of an anhydrous emulsion.

In exemplary embodiments, the cosmetic compositions may possess, for example, a continuous fatty phase that may contain less than about 10% by weight of water, for example, less than about 5% by weight of water.

The cosmetic compositions according to exemplary embodiments may be anhydrous: that is, exemplary cosmetic compositions may contain less than about 5%, such as less than about 3%, less than about 2%, or less than about 1% of water relative to the total weight of the composition. Such exemplary cosmetic compositions may then be in the form in particular of oily gels, oily liquids, pastes or sticks or may be in the form of a vesicular dispersion containing ionic and/or nonionic liquids.

Film Formers

As used herein, a “film-forming polymer” encompasses polymers capable of forming, alone or in the presence of an auxiliary film-forming agent, continuous films that adhere to substrates, such as to keratin materials. Such film forming polymers may form cohesive films or, in embodiments, films having cohesion and mechanical properties such that the film can be isolated from the substrate.

Among the film-forming polymers that may be used in the cosmetic compositions of exemplary embodiments of the present invention, mention may be made of synthetic polymers, of free-radical or polycondensate type, polymers of natural origin, and mixtures thereof. As film-forming polymer, mention may be made in particular of acrylic polymers, polyurethanes, polyesters, polyamides, polyureas, and cellulosic polymers, such as nitrocellulose.

In an exemplary embodiment, the film-forming polymer is at least one polymer selected from the group consisting of:
film-forming polymers soluble in the liquid fatty phase, such as fat-soluble polymers, when the liquid fatty phase comprises at least one oil;
film-forming polymers dispersible in the liquid fatty phase, such as polymers in the form of non-aqueous dispersions of polymer particles, for example, dispersions in silicone oils or hydrocarbon oils; in embodiments, non-aqueous polymer dispersions comprise aforementioned surface-stabilized polymer particles;
aqueous dispersions of particles of film-forming polymers, or “latexes”; in such embodiments, the composition must include, in addition to the liquid fatty phase, an aqueous phase;
water-soluble film-forming polymers; in such embodiments, the composition must include, besides the liquid fatty phase, an aqueous phase.

I. Film Former Dispersible in the Liquid Fatty Phase
1) Graft Polymer

The cosmetic compositions according to exemplary embodiments of the invention may comprise, as film former, a dispersion of particles of a graft ethylenic polymer in a liquid fatty phase.

As used herein, the term “ethylenic” polymer encompasses polymers obtained by polymerization of ethylenically unsaturated monomers.

Dispersions of graft ethylenic polymer in embodiments are essentially free of stabilizing polymers that are different from the graft polymer, such as those described in EP 749 747 and described hereinbelow, and the particles of graft ethylenic polymer are therefore not surface-stabilized with such additional stabilizing polymers. The graft polymer is therefore dispersed in the liquid fatty phase in the absence of additional surface stabilizer for the particles.

As used herein, the term “graft” polymer encompasses polymers having skeletons comprising at least one side chain that is pendent or located at the end of a chain.

In exemplary embodiments, the graft ethylenic polymer comprises an ethylenic skeleton that is insoluble in said liquid fatty phase, and side chains covalently bonded to the skeleton, which are soluble in the liquid fatty phase.

The graft ethylenic polymer is, in embodiments, a non-crosslinked polymer. In particular embodiments, the polymer is obtained by polymerization of monomers comprising only one polymerizable group.

According to exemplary embodiments of the invention, the graft ethylenic polymer is a graft acrylic polymer.

The graft ethylenic polymer of exemplary embodiments may be obtained by free-radical polymerization in an organic polymerization medium:
of at least one ethylenic monomer, such as of at least one acrylic monomer and optionally of at least one additional non-acrylic vinyl monomer, to form an insoluble skeleton; and
of at least one macromonomer comprising a polymerizable end group to form the side chains, the macromonomer having a weight-average molecular mass of greater than or equal to about 200 and the content of polymerized macromonomer representing from about 0.05% to about 20% by weight of the polymer.

The liquid fatty phase of embodiments of cosmetic compositions may contain the organic polymerization medium for the graft ethylenic polymer.

The organic liquid dispersion medium, corresponding to the medium in which the graft polymer is supplied, may be identical to the polymerization medium in some exemplary embodiments.

However, the polymerization medium may be totally or partially replaced with another organic liquid medium in some exemplary embodiments. This other organic liquid medium may be added, after polymerization, to the polymerization medium. The polymerization medium is then totally or partially evaporated.

In embodiments, the liquid fatty phase may contain liquid organic compounds other than those present in the dispersion medium. These other compounds are chosen so that the graft polymer remains in dispersed form in the liquid fatty phase.

The organic liquid dispersion medium is present in the liquid fatty phase of the embodiments of the cosmetic composition according to the invention, due to the introduction of the graft polymer dispersion obtained.

The liquid fatty phase comprises, in embodiments, one or more liquid organic compounds (or oils) as defined below. In some embodiments, the liquid fatty phase is predominantly comprised of such liquid organic compounds or oils.

In particular embodiments, the liquid fatty phase is a non-aqueous liquid organic phase that is immiscible with water at room temperature (25° C.).

As used herein, the term “liquid organic compound” encompasses non-aqueous compounds that are in liquid form at room temperature (25° C.) and therefore flow under their own weight.

As used herein, the term “silicone compound” encompasses compounds containing at least one silicon atom.

Among the liquid organic compounds or oils that may be present in the liquid organic dispersion medium of exemplary embodiments, mention may be made of:
liquid organic compounds, for example, silicone or non-silicone compounds, having an overall solubility parameter according to the Hansen solubility space of less than or equal to about 18 (MPa)^{1/2} such as less than or equal to about 17 (MPa)^{1/2},
monoalcohols having an overall solubility parameter according to the Hansen solubility space of less than or equal to about 20 (MPa)^{1/2}, and
mixtures thereof.

The overall solubility parameter 6 according to the Hansen solubility space is defined in the article “Solubility parameter values” by Eric A. Grulke in the book “Polymer Handbook”, 3rd Edition, Chapter VII, pp. 519-559, by the relationship:
δ=(δ_{D}²+δ_{P}²+δ_{H}²)^{1/2}
in which
   δ_{D} represents the London dispersion forces arising from the formation of dipoles induced during molecular impacts,
   δ_{P} represents the Debye interaction forces between permanent dipoles, and
   δ_{H} represents the forces of specific interactions (such as hydrogen bonding, acid/base, donor/acceptor, etc.).

The definition of solvents in the solubility space according to Hansen is described in the article by C. M. Hansen: “The three dimensional solubility parameters”, J. Paint Technol. 39, 105 (1967).

Among the liquid organic compounds having an overall solubility parameter according to the Hansen solubility space of less than or equal to about 18 (MPa)^{1/2}, mention may be made of liquid fatty substances, such as oils, which may be chosen from natural or synthetic oils, carbon oils, hydrocarbon oils, fluoro oils and silicone oils, which are optionally branched, alone or as a mixture.

Among these oils, mention may be made of plant oils formed from fatty acid esters and from polyols, in particular triglycerides, such as sunflower oil, sesame oil or rapeseed oil, or esters derived from acids or alcohols containing a long chain (i.e. a chain containing from 6 to 20 carbon atoms), in particular the esters of formula RCOOR′ in which R represents a higher fatty acid residue containing from 7 to 19 carbon atoms and R′ represents a hydrocarbon chain containing from 3 to 20 carbon atoms, such as palmitates, adipates and benzoates, in particular diisopropyl adipate.

Mention may also be made of linear, branched and/or cyclic alkanes which may be volatile, for example, liquid paraffin, liquid petroleum jelly or hydrogenated polyisobutylene, isododecane or “ISOPARS”, volatile isoparaffins. Mention may also be made of esters, ethers and ketones.

Mention may also be made of silicone oils, such as polydimethylsiloxanes and polymethylphenylsiloxanes, optionally substituted with aliphatic and/or aromatic groups that may be optionally fluorinated, substituted or with functional groups such as hydroxyl, thiol and/or amine groups; and volatile silicone oils, which may be cyclic in embodiments.

In particular embodiments, the oils may be chosen from volatile and/or non-volatile, optionally branched silicone oils.

As non-silicone liquid organic compounds with an overall solubility parameter according to the Hansen solubility space of less than or equal to about 18 (MPa)^{1/2} for inclusion in embodiments, mention may be made in particular of:
linear, branched or cyclic esters containing at least 6 carbon atoms, such as linear, branched or cyclic esters containing 6 to 30 carbon atoms;
ethers containing at least 6 carbon atoms, such as ethers containing 6 to 30 carbon atoms; and
ketones containing at least 6 carbon atoms, such as ketones containing 6 to 30 carbon atoms.

As used herein, “liquid monoalcohols having an overall solubility parameter according to the Hansen solubility space of less than or equal to about 20 (MPa)^{1/2}” encompasses aliphatic fatty liquid monoalcohols containing from 6 to 30 carbon atoms in which the hydrocarbon chain not comprising a substitution group. Monoalcohols according to exemplary embodiments of the invention include oleyl alcohol, decanol, octyldodecanol and linoleyl alcohol.

When the liquid fatty phase of the cosmetic composition according to exemplary embodiments the invention is a non-silicone liquid fatty phase, the macromonomers present in the graft polymer may be carbon macromonomers as described below.

In particular embodiments, when the liquid fatty phase of the cosmetic composition is a non-silicone liquid fatty phase, the graft polymer present in the composition may be a non-silicone graft polymer.

As used herein, the term “non-silicone graft polymer” encompasses graft polymers predominantly containing a carbon macromonomer and optionally containing not more than about 7% by weight of the silicone macromonomer, such as graft polymers containing not more than about 5% by weight of silicone macromonomer, or graft polymers that are free of silicone macromonomer.

When the liquid fatty phase of the cosmetic composition according to embodiments is a silicone liquid fatty phase, the macromonomers present in the graft polymer may be silicone macromonomers as described below.

In particular embodiments, when the liquid fatty phase is a silicone liquid fatty phase, the graft polymer present in the composition may be a silicone graft polymer.

As used herein, the term “silicone graft polymer” encompasses graft polymers predominantly containing a silicone macromonomer and optionally containing not more than 7% by weight of the silicone macromonomer, and graft polymers containing not more than 5% by weight of carbon macromonomer, or graft polymers that are free of carbon macromonomer.

a) Monomers

In embodiments, the choice of monomers constituting the skeleton of the polymer, of macromonomers, the molecular weight of the polymer, and the proportion of the monomers and macromonomers may be made as a function of the liquid organic dispersion medium to obtain a dispersion of particles of graft polymers, such as a stable dispersion.

As used herein, the term “stable dispersion” encompasses dispersions that are not liable to form solid deposits or to undergo liquid/solid phase separation, such as after centrifugation, for example at 4000 rpm for 15 minutes.

The graft ethylenic polymer forming the particles in dispersion may comprise, in exemplary embodiments, comprises a skeleton that is insoluble in the dispersion medium and a portion that is soluble in the dispersion medium.

The graft ethylenic polymer may be a random polymer.

According to exemplary embodiments of the invention, the term “graft ethylenic polymer” encompasses a polymer that may be obtained by free-radical polymerization:
of one or more ethylenic monomer(s);
with one or more macromonomer(s), in an organic polymerization medium.

According to embodiments of the invention, the term “graft acrylic polymer” encompasses polymers that may be obtained by free-radical polymerization:
of one or more acrylic monomer(s), and optionally of one or more additional non-acrylic vinyl monomer(s);
with one or more macromonomer(s), in an organic polymerization medium.

In exemplary embodiments, the acrylic monomers represent from about 50% to about 100% by weight of the silicone macromonomer, such as graft polymers containing and preferably not more than about 5% by weight of silicone macromonomer, or even being graft polymers that are free of silicone macromonomer, such as from about 55% to about 100% by weight, from about 55% to about 95% by weight, from about 60% to about 100% by weight, or about 60% to about 90% by weight.

In particular embodiments, the acrylic monomers may be chosen from monomers whose homopolymer is insoluble in the dispersion medium under consideration, i.e. the homopolymer is in solid (or non-dissolved) form at a concentration of greater than or equal to about 5% by weight at room temperature (20° C.) in the dispersion medium.

According to exemplary embodiments of the invention, “macromonomer containing a polymerizable end group” encompasses any polymer comprising, on only one of its ends, a polymerizable end group capable of reacting during the polymerization reaction with acrylic monomers and optionally or reacting with the additional non-acrylic vinyl monomers constituting the skeleton. The macromonomer enables the formation of side chains of the graft acrylic polymer. The polymerizable group of the macromonomer may be, in embodiments, an ethylenically unsaturated group capable of free-radical polymerization with the monomers constituting the skeleton.

As used herein, the term “carbon macromonomer” encompasses non-silicone macromonomers and oligomeric macromonomers obtained by polymerization of ethylenically unsaturated non-silicone monomer(s), and mainly by polymerization of acrylic and/or non-acrylic vinyl monomers.

As used herein, the term “silicone macromonomer” encompasses organopolysiloxane macromonomers, such as polydimethylsiloxane macromonomers.

In particular embodiments, the macromonomer may be chosen from macromonomers whose homopolymer is soluble in the dispersion medium under consideration, i.e. fully dissolved at a concentration of greater than or equal to about 5% by weight and at room temperature in the dispersion medium.

Thus, the graft acrylic polymer of exemplary embodiments comprises a skeleton (or main chain) consisting of a sequence of acrylic units resulting from the polymerization of one or more acrylic monomers and of side chains (or grafts) that are derived from the reaction of the macromonomers and that are covalently bonded to said main chain.

The skeleton (or main chain) is insoluble in the dispersion medium of exemplary embodiments, whereas the side chains (or grafts) are soluble in said dispersion medium.

As used herein, the term “acrylic monomers” encompasses monomers selected from (meth)acrylic acid, (meth)acrylic acid esters (also known as (meth)acrylates), and (meth)acrylic acid amides (also known as (meth)acrylamides).

As acrylic monomers that may be used to constitute the insoluble skeleton of the polymer of exemplary embodiments, mention may be made, alone or as a mixture, of the following monomers, and also the salts thereof:
(i) the (meth)acrylates of formula (VIII): 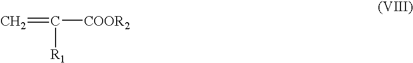
in which:
R₁ denotes a hydrogen atom or a methyl group;
R₂ represents a group chosen from:
a linear or branched alkyl group containing from 1 to 6 carbon atoms, these groups may comprise one or more heteroatoms selected from O, N and S in the alkyl chain; and/or these groups may comprise one or more substituents selected from —OH, halogen atoms (F, Cl, Br or I) and —NR′R″ with R′ and R″, which may be identical or different, chosen from linear or branched C₁-C₄ alkyls; and/or groups substituted with at least one polyoxyalkylene group, such as C₂-C₄ alkylene, for example, polyoxyethylene groups and/or polyoxypropylene groups, said polyoxyalkylene group consisting of a repetition of 5 to 30 oxyalkylene units;
a cyclic alkyl group containing from 3 to 6 carbon atoms, said group optionally comprising one or more heteroatoms selected from O, N and S in the alkyl chain, and/or optionally comprising one or more substituents selected from OH and halogen atoms (F, Cl, Br or I).

Examples of R₂ that may be mentioned include the methyl, ethyl, propyl, butyl, isobutyl, methoxyethyl, ethoxyethyl, methoxypolyoxyethylene (350 OE), trifluoroethyl, 2-hydroxyethyl, 2-hydroxypropyl, dimethylaminoethyl, diethylaminoethyl or dimethylaminopropyl groups;
(ii) the (meth)acrylamides of formula (IX): 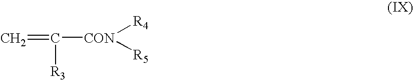
in which:
R₃ denotes a hydrogen atom or a methyl group;
R₄ and R₅, which may be identical or different, represent a hydrogen atom or a linear or branched alkyl group containing from 1 to 6 carbon atoms, which may comprise one or more substituents selected from —OH, halogen atoms (F, Cl, Br or I) and —NR′R″ with R′ and R″, which may be identical or different, chosen from linear or branched C₁-C₄ alkyls; or
R₄ represents a hydrogen atom and R₅ represents a 1,1-dimethyl-3-oxobutyl group.

As examples of alkyl groups that can constitute R₄ and R₅, mention may be made of n-butyl, t-butyl, n-propyl, dimethylaminoethyl, diethylaminoethyl and dimethylaminopropyl;
(iii) (meth)acrylic monomers comprising at least one carboxylic acid, phosphoric acid or sulphonic acid function, such as acrylic acid, methacrylic acid or acrylamidopropanesulphonic acid.

In exemplary embodiments, such acrylic monomers may include methyl, ethyl, propyl, butyl and isobutyl (meth)acrylates; methoxyethyl or ethoxyethyl (meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate; dimethylaminopropylmethacrylamide; and the salts thereof; and mixtures thereof.

In particular embodiments, the acrylic monomers may be chosen from methyl acrylate, methoxyethyl acrylate, methyl methacrylate, 2-hydroxyethyl methacrylate, acrylic acid and dimethylaminoethyl methacrylate, and mixtures thereof.

In exemplary embodiments, such additional non-acrylic vinyl monomers may include:
vinyl esters of the formula below:
   R₆—COO—CH═CH₂
in which:
R₆ represents a linear or branched alkyl group containing from 1 to 6 atoms, a cyclic alkyl group containing from 3 to 6 carbon atoms and/or an aromatic group, for example of benzene, anthracene or naphthalene type;
non-acrylic vinyl monomers comprising at least one carboxylic acid, phosphoric acid or sulphonic acid function, such as crotonic acid, maleic anhydride, itaconic acid, fumaric acid, maleic acid, styrenesulphonic acid, vinylbenzoic acid or vinylphosphoric acid, and the salts thereof;
non-acrylic vinyl monomers comprising at least one tertiary amine function, such as 2-vinylpyridine or 4-vinylpyridine;
and mixtures thereof.

In exemplary embodiments, the acrylic monomers present in the graft polymer comprise at least (meth)acrylic acid and at least one monomer chosen from the (meth)acrylates and (meth)acrylamides described previously in points (i) and (ii). In particular embodiments, the acrylic monomers comprise at least (meth)acrylic acid and at least one monomer chosen from C₁-C₃ alkyl (meth)acrylates. (Meth)acrylic acid may be present in a content of at least about 5% by weight relative to the total weight of the polymer, for of at least about 10% by weight, or of at least about 15% by weight, and may be present in a range of from about 5% to about 80% by weight, such as from about 10% to about 70% by weight, or from about 15% to about 60% by weight, relative to the total weight of the polymer.

Among the salts that may used in exemplary embodiments include salts obtained by neutralization of acid groups with inorganic bases, such as sodium hydroxide, potassium hydroxide or ammonium hydroxide, or organic bases of alkanolamine type, for instance monoethanolamine, diethanolamine, triethanolamine or 2-methyl-2-amino-1-propanol.

In embodiments, the salts include those formed by neutralization of tertiary amine units, for example using a mineral or organic acid. Among the mineral acids that may be used in embodiments are sulphuric acid, hydrochloric acid, hydrobromic acid, hydriodic acid, phosphoric acid or boric acid. Among the organic acids that may be used in embodiments are acids comprising one or more carboxylic, sulphonic or phosphonic groups. Such organic acids may be linear, branched or cyclic aliphatic acids, or alternatively aromatic acids, and may also comprise one or more heteroatoms selected from O and N, for example in the form of hydroxyl groups. Acetic acid or propionic acid, terephthalic acid, and citric acid and tartaric acid may also be mentioned.

According to exemplary embodiments of the invention, the graft ethylenic polymer contains no additional non-acrylic vinyl monomers as described above, and the insoluble skeleton of the graft ethylenic polymer is formed solely from acrylic monomers as described previously.

These non-polymerized acrylic monomers may be soluble in the dispersion medium of embodiments, but the polymer formed with these monomers is insoluble in the dispersion medium.

According to exemplary embodiments of the invention, the graft ethylenic polymer may be obtained by free-radical polymerization in an organic polymerization medium:
of a main acrylic monomer selected from C₁-C₃ alkyl (meth)acrylates, alone or as a mixture, and optionally of one or more additional acrylic monomers chosen from (meth)acrylic acid, methacrylic acid and alkyl (meth)acrylates of formula (X) defined below, and salts thereof, to form said insoluble skeleton; and
of at least one silicone macromonomer comprising a polymerizable end group, as defined previously.

Main acrylic monomers that may be used include in exemplary embodiments methyl acrylate, methyl methacrylate, ethyl acrylate, ethyl methacrylate, n-propyl acrylate, n-propyl methacrylate, isopropyl acrylate and isopropyl methacrylate, and mixtures thereof.

In particular embodiments, main acrylic monomers include methyl acrylate, methyl methacrylate and ethyl methacrylate.

In embodiments additional acrylic monomers may be chosen from:
(meth)acrylic acid and salts thereof,
the (meth)acrylates of formula (X), and salts thereof: 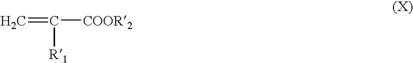
in which:
R′₁ denotes a hydrogen atom or a methyl group;
R′₂ represents
a linear or branched alkyl group containing from 1 to 6 carbon atoms and comprises one or more oxygen atoms in the alkyl chain and/or comprises one or more substituents chosen from —OH, halogen atoms (F, Cl, Br or I) and —NR′R″, with R′ and R″, which may be identical or different, that are chosen from linear or branched C₁-C₃ alkyls;
a cyclic alkyl group containing from 3 to 6 carbon atoms and optionally comprises one or more oxygen atoms and/or optionally comprising one or more substituents selected from OH and halogen atoms (F, Cl, Br or I);
and mixtures thereof.

Examples of R′₂ that may be used in exemplary embodiments include the methoxyethyl, ethoxyethyl, trifluoroethyl; 2-hydroxyethyl, 2-hydroxypropyl, dimethylaminoethyl, diethylaminoethyl and dimethylaminopropyl groups.

In particular embodiments, these additional acrylic monomers may be chosen from (meth)acrylic acid, methoxyethyl or ethoxyethyl (meth)acrylate; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate and 2-hydroxyethyl acrylate, the salts thereof, and mixtures thereof.

Acrylic acid and methacrylic acid may be used in certain embodiments.

b) Macromonomers

The macromonomers of exemplary embodiments comprise, at one of the ends of the molecular chain, a polymerizable end group capable of reacting with acrylic monomers during polymerization and optionally with additional vinyl monomers, to form the side chains of the graft ethylenic polymer. The polymerizable end group may, in particular embodiments, be a vinyl or (meth)acrylate (or (meth)acryloxy) group.

The macromonomers may be selected, in embodiments, from macromonomers whose homopolymer has a glass transition temperature (Tg) of less than or equal to about 25° C., for example ranging from about −100° C. to about 25° C. or from about −80° C. to about 0° C.

In embodiments, macromonomers have a weight-average molecular mass of greater than or equal to about 200, such as greater than or equal to about 300, greater than or equal to about 500 or greater than about 600.

In particular exemplary embodiments, the macromonomers have a weight-average molecular mass (Mw) ranging from about 200 to about 100,000, such as from about 500 to about 50,000, from about 800 to about 20,000, ranging from about 800 to about 10,000, or ranging from about 800 to about 6000.

Herein, the weight-average (Mw) and number-average (Mn) molar masses are determined by liquid gel permeation chromatography (THF solvent, calibration curve established with linear polystyrene standards, refractometric detector).

Carbon macromonomers that may be used in particular embodiments include:
(i) homopolymers and copolymers of linear or branched C₈-C₂₂ alkyl (meth)acrylate, containing a polymerizable end group chosen from vinyl or (meth)acrylate groups, such as poly(2-ethylhexyl acrylate) macromonomers with a mono(meth)acrylate end group; poly(dodecyl acrylate) or poly(dodecyl methacrylate) macromonomers with a mono(meth)acrylate end group; poly(stearyl acrylate) and poly(stearyl methacrylate) macromonomers with a mono(meth)acrylate end group. Such macromonomers are described in particular in EP 895 467 and EP 96 459, and in Gillman K. F., Polymer Letters, Vol. 5, page 477-481 (1967). In particular embodiments, macromonomers based on poly(2-ethylhexyl acrylate) or poly(dodecyl acrylate) with a mono(meth)acrylate end group may be used.
(ii) polyolefins containing an ethylenically unsaturated end group, such as polyolefins containing a (meth)acrylate end group. Examples of such polyolefins that include the following macromonomers that include a (meth)acrylate end group: polyethylene macromonomers, polypropylene macromonomers, macromonomers of polyethylene/polypropylene copolymer, macromonomers of polyethylene/polybutylene copolymer, polyisobutylene macromonomers; polybutadiene macromonomers; polyisoprene macromonomers; polybutadiene macromonomers; poly(ethylene/butylene)-polyisoprene macromonomers. Such macromonomers are described in particular in U.S. Pat. No. 5,625,005, which mentions ethylene/butylene and ethylene/propylene macromonomers containing a (meth)acrylate reactive end group.

Mention may also be made of the poly(ethylene/butylene) methacrylate such as that sold under the name KRATON LIQUID L-1253 by KRATON POLYMERS.

Silicone macromonomers that may be mentioned for use in exemplary embodiments also include polydimethylsiloxanes containing mono(meth)acrylate end groups, such as silicone macromonomers of formula (XI) below: 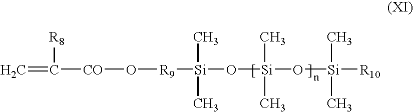
in which:
   R₈ denotes a hydrogen atom or a methyl group;
   R₉ denotes a divalent hydrocarbon group containing from 1 to 10 carbon atoms and optionally contains one or two ether bonds —O—;
   R₁₀ denotes an alkyl group containing from 1 to 10 carbon atoms, such as from 2 to 8 carbon atoms; and
   n denotes an integer in the range of from 1 to about 300, such as an integer in the range of from about 3 to about 200 or an integer in the range of from about 5 to about 100.

Silicone macromonomers that may be used in embodiments include monomethacryloxypropyl polydimethylsiloxanes, such as those sold under the name PS560-K6 by UNITED CHEMICAL TECHNOLOGIES INC. (UCT) or under the name MCR-M17 by GELEST INC.

In some exemplary embodiments, the polymerized macromonomer (constituting the side chains of the graft polymer) represents from about 0.1% to about 15% by weight, such as from about 0.2% to about 10% by weight or from about 0.3% to about 8% by weight, of the total weight of the polymer.

In particular embodiments, the graft ethylenic polymer dispersed in a non-silicone liquid fatty phase is a graft ethylenic polymer that is obtained by polymerization:
of methyl acrylate and of a polyethylene/polybutylene macromonomer containing a methacrylate end group (such as KRATON L-1253), for example, in a solvent chosen from isododecane, isononyl isononanoate, octyldodecanol, diisostearyl malate or a C₁₂-C₁₅ alkyl benzoate (such as Finsolv TN);
of methoxyethyl acrylate and of a polyethylene/polybutylene macromonomer containing a methacrylate end group (such as KRATON L-1253), for example, in isododecane;
of methyl acrylate/methyl methacrylate monomers and of a polyethylene/polybutylene macromonomer containing a methacrylate end group (such as KRATON L-1253), for example in isododecane;
of methyl acrylate/acrylic acid monomers and of a polyethylene/polybutylene macromonomer containing a methacrylate end group (such as KRATON L-1253), for example in isododecane;
of methyl acrylate/dimethylaminoethyl methacrylate monomers and of a polyethylene/polybutylene macromonomer containing a methacrylate end group (such as KRATON L-1253), for example in isododecane;
of methyl acrylate/2-hydroxyethyl methacrylate monomers and of a polyethylene/polybutylene macromonomer containing a methacrylate end group (such as KRATON L-1253), for example in isododecane.

In particular embodiments, the graft acrylic polymer dispersed in a silicone liquid fatty phase is obtained by polymerization:
of methyl acrylate and of the monomethacryloyloxypropyl polydimethylsiloxane macromonomer with a weight-average molecular weight ranging from about 800 to about 6000, for example in decamethylcyclopentasiloxane or phenyl trimethicone;
of methyl acrylate, acrylic acid and the monomethacryloxypropyl polydimethylsiloxane macromonomer with a weight-average molecular weight ranging from about 800 to about 6000, for example in decamethylcyclopentasiloxane or phenyl trimethicone.

The weight-average molecular mass (Mw) of the graft polymer of embodiments may be between about 10,000 and about 300,000, such as between about 20,000 and about 200,000 or between about 25,000 and about 150,000.

By virtue of the abovementioned characteristics, in a given organic dispersion medium, the polymers of exemplary embodiments have the capacity of folding over on themselves to form particles of substantially spherical shape, which have deployed side chains on the periphery that ensure particle stability. Such particles do not aggregate in the dispersion medium, are self-stabilized, and form particularly stable polymer particle dispersions.

In particular embodiments, the graft ethylenic polymers of the dispersion are capable of forming nanometer-sized particles, with a mean size ranging from about 10 to about 400 nm, such as from about 20 to about 200 nm.

As a result of this very small size, the graft polymer particles of embodiments are particularly stable and have little susceptibility to aggregation.

The dispersion of graft polymer in exemplary embodiments may thus be a stable dispersion that does not form sediments when held at room temperature (25° C.) for an extended period (for example 24 hours).

In particular embodiments, the dispersion of graft polymer particles has a solids content (or dry extract) of polymer of from about 40% to about 70% by weight of solids, such as from about 45% to about 65% by weight.

c) Preparation Process

The graft polymer particle dispersion of exemplary embodiments may be prepared via a process that includes a free-radical copolymerization step, in an organic polymerization medium, of one or more acrylic monomers as defined above with one or more macromonomers as defined above.

As mentioned previously, the liquid organic dispersion medium of embodiments may be identical to or different from the polymerization medium.

In exemplary embodiments, copolymerization may be performed, conventionally, in the presence of a polymerization initiator. The polymerization initiators may be free-radical initiators. In general, such a polymerization initiator may be chosen from organic peroxide compounds, such as dilauroyl peroxide, dibenzoyl peroxide or tert-butyl peroxy-2-ethylhexanoate; diazo compounds, such as azobisisobutyronitrile; or azobisdimethylvaleronitrile.

The copolymerization reaction of embodiments may also be initiated using photoinitiators or with radiation such as UV or neutrons, or with plasma.

In general, the copolymerization reaction process of embodiments entails introducing at least a portion of the organic polymerization medium; a portion of the additional acrylic and/or vinyl monomers, which will constitute the insoluble skeleton after polymerization; all of the macromonomer, which will constitute the side chains of the polymer; and a portion of the polymerization initiator into a reactor of a suitable size for the amount of polymer to be prepared. At this stage of introduction, the reaction medium forms a relatively homogeneous medium.

The reaction medium is then stirred and heated to obtain polymerization of the monomers and macromonomers. The initially homogeneous and clear medium leads to a dispersion of milky appearance. A mixture of the remaining portion of monomers and of polymerization initiator is then added. After the mixture is heated with stirring for a period of time, the medium stabilizes in the form of a milky dispersion, which comprises polymer particles, stabilized in the polymerization medium due to the presence of polymer side chains that are soluble in the dispersion medium.

The graft polymer may be present in the composition according to exemplary embodiments of the invention in a solids content (or active material content) ranging from about 1% to about 70% by weight, such as from about 5% to about 60% by weight, from about 6% to about 45% by weight, or from about 8% to about 40% by weight, relative to the total weight of the composition.

2) Block Polymer

The composition according to exemplary embodiments of the invention may contain, as film former, a linear block ethylenic polymer, referred to hereinbelow as a “block polymer”, the particular structure of which being as described below.

As used herein, the term “block” polymer encompasses polymers comprising at least two different blocks, for example polymers that comprise at least three different blocks.

The polymer of exemplary embodiments is a polymer of linear structure. In contrast, a polymer of non-linear structure is, for example, a polymer of branched, star or graft structure, or of other structure.

In exemplary embodiments, the block polymer is free of styrene. As used herein, the term “polymer free of styrene” encompasses polymers containing less than about 10% by weight relative to the total weight of the polymer, such as less than about 5% by weight, less than about 2% by weight, or less than about 1% by weight of styrenic monomers, such as styrene and/or styrene derivatives, such as methylstyrene, chlorostyrene or chloromethylstyrene. In embodiments, the polymer free of styrene contains no styrene monomer.

In particular embodiments, the block polymer comprises at least one first block and at least one second block that have different glass transition temperatures (Tg), and the first and second blocks are linked together via an intermediate block that includes at least one constituent monomer of the first block and at least one constituent monomer of the second block.

As used herein, the term “at least one block” encompasses one or more blocks.

The intermediate block of embodiments is a block comprising at least one constituent monomer of the first block and at least one constituent monomer of the second block of the polymer, allowing these blocks to be “compatibilized”.

As used herein, the terms “first” and “second” blocks do not in any way condition the order of said blocks in the structure of the block polymer. Rather, the terms “first” and “second” blocks merely differentiate the constituent monomers.

In exemplary embodiments, the first and second blocks of the block polymer are mutually incompatible.

As used herein, the term “mutually incompatible blocks” indicates that the mixture formed from the polymer corresponding to the first block and of the polymer corresponding to the second block is not miscible in the organic liquid (the major component by weight) of the liquid fatty phase, at room temperature (25° C.) and atmospheric pressure (10⁵ Pa), for polymer mixture contents greater than or equal to about 5% by weight, relative to the total weight of the mixture (polymers and solvent), for polymer mixtures in which:
i) the polymers corresponding to the first block and polymers corresponding to the second block are present in the mixture in a content in weight ratios ranging from about 10/90 to about 90/10, and that
ii) each of the polymers corresponding to the first and second blocks has an average (weight-average or number-average) molecular mass equal to that of the block polymer ±about 15%.

In embodiments in which the liquid fatty phase comprises a mixture of organic liquids, such as two or more organic liquids present in identical mass proportions, said polymer mixture is immiscible in at least one of them.

In embodiments in which the liquid fatty phase comprises only one organic liquid, this liquid is the predominant organic liquid.

In embodiments, the block polymer comprises no silicon atoms in the polymer skeleton. As used herein, the term “skeleton” indicates the main chain of the polymer, as opposed to the pendent side chains.

In particular embodiments, an active material content of at least about 1% by weight of the block polymer is not soluble in water or in a mixture of water and linear or branched lower monoalcohols containing from 2 to 5 carbon atoms, for instance ethanol, isopropanol or n-propanol, at room temperature (25° C.), unless the pH is modified.

In particular embodiments, the block polymer is a non-elastomeric polymer. As used herein, the term “non-elastomeric polymer” encompasses polymers which stretches when subjected to a strain ((for example, by 30% relative to the initial polymer length), but does not return to a length substantially identical to the initial length when the strain ceases.

a) Recovery Test

More specifically, the term “non-elastomeric polymer” encompasses polymers with an instantaneous recovery Rᵢ<50% and a delayed recovery R₂ₕ<70% after having been subjected to a 30% elongation. In particular embodiments, Rᵢ is <30% and R₂ₕ<50 for the non-elastomeric polymer.

In embodiments, the non-elastomeric nature of the polymer is determined according to the following protocol:

A polymer film is prepared by pouring a solution of the polymer in a Teflon-coated mold, and drying for 7 days in an environment conditioned at 23±5° C. and 50±10% relative humidity.

A film about 100 μm thick is thus obtained, from which are cut rectangular specimens (for example using a punch) 15 mm wide and 80 mm long.

This sample is subjected to a tensile stress using a machine sold under the reference ZWICK, under the same temperature and humidity conditions as for the drying.

The specimens are pulled at a speed of 50 mm/min and the distance between the jaws is 50 mm, which corresponds to the initial length (l₀) of the specimen.

Instantaneous recovery Rᵢ is determined in the following manner:
the specimen is pulled by 30% (εₘₐₓ), (i.e. about 0.3 times its initial length (l₀).
the strain is released by applying a return speed equal to the tensile speed, i.e. 50 mm/min, and the residual elongation of the specimen is measured as a percentage, after returning to zero strain (εᵢ).

The percentage instantaneous recovery (Rᵢ) is given by the following formula:
Rᵢ=(εₘₐₓ−εᵢ)/εₘₐₓ)×100

To determine the delayed recovery, the percentage residual elongation of the specimen (ε₂ₕ) is measured 2 hours after returning to zero strain.

The percentage delayed recovery (R₂ₕ) is given by the following formula:
R₂ₕ=(εₘₐₓ−ε₂ₕ)/εₘₐₓ)×100

For example, in a particular embodiment, the polymer may have an instantaneous recovery Rᵢ of about 10% and a delayed recovery R₂ₕ of about 30%.

In embodiments, the block polymer has a polydispersity index I of greater than about 2, for example, in a range of from about 2 to about 9; of greater than or equal to about 2.5, for example, in a range of from about 2.5 to about 8, or of greater than or equal to about 2.8, for example, in a range of from about 2.8 to about 6.

In embodiments, polydispersity index I of the block polymer is equal to the ratio of the weight-average mass Mw to the number-average mass Mn.

The weight-average molar mass (Mw) and number-average molar mass (Mn) may be determined, in embodiments, by gel permeation liquid chromatography (THF solvent, calibration curve established with linear polystyrene standards, refractometric detector).

In exemplary embodiments, the weight-average mass (Mw) of the block polymer may be less than or equal to about 300 000, such as, for example, in a range of from about 35,000 to about 200,000 or in a range of from about 45,000 to about 150,000.

In exemplary embodiments, the number-average mass (Mn) of the block polymer may be less than or equal to about 70,000, such as, for example, in a range of from about 10,000 to about 60,000 or in a range of from about 12,000 to about 50,000.

Each block or sequence of the block polymer is derived, in embodiments, from one type of monomer or from several different types of monomer. This means that each block for a given embodiment may consist of a homopolymer or a copolymer that may in turn be random or alternating.

In exemplary embodiments, the intermediate block, which includes at least one constituent monomer of the first block and at least one constituent monomer of the second block of the block polymer, is a random polymer.

In particular embodiments, the intermediate block is derived essentially from constituent monomers of the first block and of the second block.

As used herein, the term “essentially” indicates at least about 85%, such as at least about 90%, at least about 95% or about 100%.

In exemplary embodiments, the intermediate block has a glass transition temperature Tg that is between the glass transition temperatures of the first and second blocks.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:
1/Tg=Σᵢ( [symbol] ᵢ/Tgᵢ),
in which [symbol] ᵢ is the mass fraction of the monomer i in the block under consideration and Tgᵢ is the glass transition temperature of the homopolymer of the monomer i.

Unless otherwise indicated, the Tg values for the first and second blocks in embodiments are theoretical Tg values.

The difference between the glass transition temperatures of the first and second blocks of exemplary embodiments is generally greater than about 10° C., such as greater than about 20° C. or greater than about 30° C.

b) Blocks of the Polymer

In particular embodiments, the first block of the block polymer may be selected from:
a) a block with a Tg of greater than or equal to about 40° C.,
b) a block with a Tg of less than or equal to about 20° C.,
c) a block with a Tg of between about 20 and about 40° C.,
and the second block may be selected from a category a), b) or c) different from the first block.

As used herein, the expression “between . . . and . . . ” indicates a range of values for which the limits mentioned are excluded, i.e. a range not including the indicated endpoints, and the expression “from . . . to . . . ” and “ranging from . . . to . . . ” indicates a range of values for which the limits are included, i.e. an inclusive range.

a) Block with a Tg of Greater than or Equal to 40° C.

In exemplary embodiments, the block with a Tg of greater than or equal to about 40° C. has, for example, a Tg ranging from about 40 to about 150° C.; the block with a Tg of greater than or equal to about 50° C. has, for example, a Tg ranging from about 50° C. to about 120° C.; and the block with a Tg of greater than or equal to about 60° C. has, for example, a Tg ranging from about 60° C. to about 120° C.

In embodiments, the block with a Tg of greater than or equal to about 40° C. may be a homopolymer or a copolymer.

In embodiments in which the block having a Tg greater than or equal to about 40° C. is a homopolymer, the block is derived from monomers for which the homopolymers glass transition temperatures of greater than or equal to about 40° C. This first block, in embodiments, may be a homopolymer consisting of only one type of monomer for which the Tg of the corresponding homopolymer is greater than or equal to about 40° C.

In embodiments in which the first block is a copolymer, the first block may be totally or partially derived from one or more monomers, which are chosen to form a copolymer having a Tg greater than or equal to about 40° C. Such an exemplary copolymer may comprise, for example:
monomers that form homopolymers having Tg values of greater than or equal to about 40° C., greater than or equal to about 50° C., or greater than or equal to about 60° C., for example such homopolymers may have a Tg ranging from about 40 to about 150° C., from about 50° C. to about 120° C. or from about 60° C. to about 120° C., and
monomers that form homopolymers having Tg values of less than about 40° C., which may be chosen from monomers with a Tg of between about 20 and about 40° C. and/or monomers with a Tg of less than or equal to 20° C., less than about 15° C. or less than about 10° C., such as, for example, monomers having a Tg ranging from about −100 to about 20° C., from about −80° C. to about 15° C. or from about −50° C. to about 0° C., as described below.

In particular embodiments, monomers that form homopolymers have a glass transition temperature of greater than or equal to about 40° C. are chosen from the following monomers, also known as main monomers:
methacrylates of formula (XII):
   CH₂═C(CH₃)—COOR₁ (XII)
in which R₁ is chosen from linear or branched unsubstituted alkyl groups containing from 1 to 4 carbon atoms, such as a methyl, ethyl, propyl or isobutyl group, and C₄ to C₁₂ cycloalkyl groups,
acrylates of formula (XIII):
   CH₂═CH—COOR₂ (XIII)
   in which R₂ is chosen from C₄ to C₁₂ cycloalkyl groups, such as isobornyl acrylate or tert-butyl groups,
(meth)acrylamides of formula (XIV): 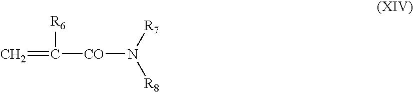
in which:
   R₇ and R₈, which may be identical or different, each are chosen from hydrogen atoms; and linear or branched C₁ to C₁₂ alkyl groups, such as an n-butyl, t-butyl, isopropyl, isohexyl, isooctyl or isononyl groups; in particular embodiments, R₇ is H and R₈ is a 1,1-dimethyl-3-oxobutyl group, and
   R′ denotes H or methyl,
   and mixtures thereof.

Examples of monomers that may be mentioned for use in embodiments include N-butylacrylamide, N-t-butylacrylamide, N-isopropylacrylamide, N,N-dimethylacrylamide and N,N-dibutylacrylamide.

Main monomers that may be used in particular exemplary embodiments are methyl methacrylate, isobutyl (meth)acrylate and isobornyl (meth)acrylate, and mixtures thereof.

b) Block with a Tg of Less than or Equal to 20° C.

In embodiments, the block having a Tg of less than or equal to about 20° C. has, for example, a Tg ranging from about −100 to about 20° C., the block having a Tg of less than or equal to about 15° C. has, for example, a Tg ranging from about −80° C. to about 15° C.; and the block having a Tg of less than or equal to about 110° C. has, for example, a Tg ranging from about −50° C. to about 0° C.

In embodiments, the block having a Tg of less than or equal to about 20° C. may be a homopolymer or a copolymer.

In embodiments in which the block having a Tg of less than or equal to about 20° C. is a homopolymer, the block is derived from monomers that can form homopolymers having glass transition temperatures of less than or equal to about 20° C. This second block, in embodiments, may be a homopolymer consisting of only one type of monomer for which the Tg of the corresponding homopolymer is less than or equal to about 20° C.

In embodiments in which the block having a Tg of less than or equal to about 20° C. is a copolymer, the block may be totally or partially derived from one or more monomers, which are chosen to form a copolymer having a Tg of less than or equal to about 20° C.

In exemplary embodiments, the copolymer block having a Tg of less than or equal to about 20° C. may comprise, for example
one or more monomers that can form a homopolymer having a Tg of less than or equal to about 20° C., less than about 15° C. or less than about 10° C.; for example, the monomers may be able to form a homopolymer having a Tg ranging from about −100° C. to about 20° C., from about −80° C. to about 15° C. or from about −50° C. to about 0° C., and
one or more monomers that can form homopolymer having a Tg of greater than about 20° C., such as monomers with a Tg of greater than or equal to about 40° C., greater than or equal to about 50° C., or greater than or equal to about 60° C., for example, the monomers may be able to form a homopolymer having a Tg ranging from about 40 to about 150° C., from about 50° C. to about 120° C. or from about 60° C. to about 120° C.; and/or monomers with a Tg of between about 20 and about 40° C., as described above.

In particular embodiments, the block having a Tg of less than or equal to about 20° C. is a homopolymer.

The monomers that can form homopolymers having a Tg of less than or equal to about 20° C. may be chosen, in exemplary embodiments, from the following monomers, or main monomers:
acrylates of formula (XV):
   CH₂═CHCOOR₃ (XV)
in which R₃ is chosen from linear or branched C₁ to C₁₂ unsubstituted alkyl group, optionally intercalating one or more heteroatoms chosen from O, N and S; however, R₃ may not be a tert-butyl group;
methacrylates of formula (XVI):
   CH₂═C(CH₃)—COOR₄ (XVI)
in which R₄ is chosen from linear or branched C₆ to Cl₂ unsubstituted alkyl groups, optionally intercalating one or more heteroatoms selected from O, N and S;
vinyl esters of formula (XVII):
   R₅—CO—O—CH═CH₂ (XVII)
in which R₅ is chosen from linear or branched C₄ to C₁₂ alkyl groups;
C₁₁ to C₁₂ vinyl alcohol ethers and C₄ to C₁₂ alcohol ethers,
N—(C₄ to C₁₂ alkyl)acrylamides, such as N-octylacrylamide,
and mixtures thereof.

The main monomers that may be used in particular exemplary embodiments for the block having a Tg of less than or equal to about 20° C. include C₁-C₁₀ alkyl acrylates, such as methyl acrylate, isobutyl acrylate and 2-ethylhexyl acrylate, and mixtures thereof; however, these C₁-C₁₀ alkyl acrylates do not include alkyl acrylates in which the alkyl group is a tert-butyl group.

c) Block with a Tg of Between 20 and 40° C.

In exemplary embodiments, the block having a Tg of between about 20 and about 40° C. may be a homopolymer or a copolymer.

In embodiments in which the block having a Tg of between about 20 and about 40° C. is a homopolymer, the block is derived from monomers, including main monomers, that can form homopolymers having glass transition temperatures of between about 20 and about 40° C. This first block, in embodiments, may be a homopolymer, consisting of only one type of monomer for which the Tg of the corresponding homopolymer ranges from about 20° C. to about 40° C.

In embodiments, monomers that can form homopolymers having a glass transition temperature of between about 20 and about 40° C. may be chosen from n-butyl methacrylate, cyclodecyl acrylate, neopentyl acrylate and isodecylacrylamide, and mixtures thereof.

In embodiments in which the block having a Tg of between about 20 and about 40° C. is a copolymer, the block is totally or partially derived from one or more monomers, including main monomers, which are chosen to form a copolymer having a Tg between about 20 and about 40° C.

In exemplary embodiments, the block having a Tg of between about 20 and about 40° C. is a copolymer totally or partially derived from:
main monomers that may form homopolymers having a Tg of greater than or equal to about 40° C., greater than or equal to about 50° C., or greater than or equal to 60° C., such as, for example, homopolymers having a Tg ranging from about 40° C. to about 150° C., from about 50 to about 120° C. or from about 60° C. to about 120° C., as described above, and/or
main monomers that can form homopolymers having a Tg of less than or equal to about 20° C., less than or equal to about 15° C., or less than or equal to about 10° C., such as, for example, homopolymers having a Tg ranging from about −100 to about 20° C., from about −80° C. to about 15° C. or from about −50° C. to about 0° C., as described above,
said monomers being chosen so that the Tg of the copolymer forming the first block is between about 20 and about 40° C.

In particular exemplary embodiments, the main monomers are chosen, for example, from methyl methacrylate, isobornyl acrylate and methacrylate, butyl acrylate and 2-ethylhexyl acrylate, and mixtures thereof.

In exemplary embodiments, the proportion of the second block having a Tg of less than or equal to about 20° C. may range from about 10% to about 85% by weight, from about 20% to about 70%, or from about 20% to about 50% by weight of the polymer.

However, in embodiments, each of the blocks may contain in small proportion at least one constituent monomer of the other block. Thus, the first block may contain at least one constituent monomer of the second block, and vice versa.

In exemplary embodiments, each of the first and/or second blocks of the block polymer may comprise, in addition to the monomers indicated above, one or more other monomers known as additional monomers, which are different from the main monomers mentioned above.

The nature and amount of this or these additional monomer(s) may be chosen to achieve the desired glass transition temperature for the block in which the additional monomer(s) re included.

c) Additional Monomer

In exemplary embodiments, additional monomers may be chosen, for example, from:
hydrophilic monomers such as:
   ethylenically unsaturated monomers comprising at least one carboxylic or sulphonic acid function, for instance:
      acrylic acid, methacrylic acid, crotonic acid, maleic anhydride, itaconic acid, fumaric acid, maleic acid, acrylamidopropanesulphonic acid, vinylbenzoic acid, vinylphosphoric acid, and salts thereof,
   ethylenically unsaturated monomers comprising at least one tertiary amine function, for instance:
      2-vinylpyridine, 4-vinylpyridine, dimethylaminoethyl methacrylate, diethylaminoethyl methacrylate and dimethylaminopropylmethacrylamide, and salts thereof,
methacrylates of formula (XVIII):
   CH₂═C(CH₃)—COOR₆ (XVIII)
in which R₆ is chosen from substituted and unsubstituted linear or branched alkyl groups containing from 1 to 4 carbon atoms, such as a methyl, ethyl, propyl or isobutyl group, in which one or more optional substituents may be selected from hydroxyl groups (for instance 2-hydroxypropyl methacrylate and 2-hydroxyethyl methacrylate) and halogen atoms (Cl, Br, I or F), such as trifluoroethyl methacrylate,
methacrylates of formula (XIX):
   CH₂═C(CH₃)—COOR₉ (XIX)
in which R₉ is chosen from substituted and unsubstituted linear or branched C₆ to C₁₂ alkyl groups optionally intercalated with one or more heteroatoms selected from O, N and S, and in which one or more optional substituents may be selected from hydroxyl groups and halogen atoms (Cl, Br, I or F);
acrylates of formula (XX):
   CH₂═CHCOOR₁₀ (XX)
in which R₁₀ is chosen from substituted linear or branched C₁ to C₁₂ alkyl groups having one or more substituents chosen from hydroxyl groups and halogen atoms (Cl, Br, I and F), such as 2-hydroxypropyl acrylate and 2-hydroxyethyl acrylate; C₁ to C₁₂ alkyl-O-POEs (polyoxyethylenes) in which the oxyethylene unit repeats 5 to 30 times, for example methoxy-POE; and polyoxyethylenated groups comprising from 5 to 30 ethylene oxide units,
ethylenically unsaturated monomers comprising one or more silicon atoms, such as methacryloxypropyltrimethoxysilane and methacryloxypropyltris(trimethylsiloxy)silane,
and mixtures thereof.

Additional monomers that may be used in exemplary embodiments include acrylic acid, methacrylic acid and trifluoroethyl methacrylate, and mixtures thereof.

According to some embodiments, the block polymer is a non-silicone polymer, i.e. a polymer free of silicon atoms.

In exemplary embodiments, the additional monomer(s) generally represent(s) an amount of less than or equal to about 30% by weight, for example, from about 1% to about 30% by weight, from about 5% to about 20% by weight or from about 7% to about 15% by weight, relative to the total weight of the first and/or second blocks.

In particular embodiments, each of the first and second blocks comprises at least one monomer selected from (meth)acrylic acid esters, and optionally at least one monomer selected from (meth)acrylic acid, and mixtures thereof.

In exemplary embodiments, each of the first and second blocks of the block polymer may be totally derived from at least one monomer chosen from acrylic acid and (meth)acrylic acid esters, and optionally at least one monomer chosen from (meth)acrylic acid, and mixtures thereof.

d) Preparation Process

The block polymer of exemplary embodiments may be prepared by free-radical solution polymerization according to the following preparation process:
a portion of the polymerization solvent is introduced into a suitable reactor and heated until the appropriate temperature for the polymerization is reached (typically between about 60 and about 120° C.),
once the polymerization temperature is reached, the constituent monomers of the first block and part of the polymerization initiator are introduced into the reactor,
after a time T corresponding to a maximum degree of conversion of 90%, the constituent monomers of the second block and the rest of the initiator are introduced,
the mixture is left to react for a time T′ (ranging from 3 to 6 hours), after which the mixture is cooled to room temperature,
the polymer dissolved in the polymerization solvent is obtained.

As used herein, the term “polymerization solvent” encompasses individual solvents and mixtures of solvents. The polymerization solvent may be chosen, in embodiments, from ethyl acetate; butyl acetate; alcohols, such as isopropanol or ethanol; and aliphatic alkanes, such as isododecane; and mixtures thereof. In particular embodiments, the polymerization solvent is a mixture of butyl acetate and isopropanol or isododecane.

According to exemplary embodiments, the block polymer comprises a first block having a Tg of greater than or equal to about 40° C., as described above in a) and a second block having a Tg of less than or equal to about 20° C., as described above in b).

In particular embodiments, the first block having a Tg of greater than or equal to about 40° C. is a copolymer derived from monomers that can form homopolymers having a glass transition temperature of greater than or equal to about 40° C., such as the monomers described above.

In exemplary embodiments, the second block having a Tg of less than or equal to about 20° C. is a homopolymer derived from monomers the can form homopolymers having a glass transition temperature of less than or equal to about 20° C., such as the monomers described above.

In particular embodiments, the proportion of the block having a Tg of greater than or equal to about 40° C. may range from about 20% to about 90%, from about 30% to about 80% or from about 50% to about 70%, by weight of the polymer.

In particular embodiments, the proportion of the block having a Tg of less than or equal to about 20° C. may range from about 5% to about 75%, from about 15% to about 50% or from about 25% to about 45%, by weight of the polymer.

In exemplary embodiments, the block polymer may comprise:
a first block having a Tg of greater than or equal to about 40° C., for example, ranging from about 85 to about 115° C., such as an isobornyl acrylate/isobutyl methacrylate copolymer,
a second block having a Tg of less than or equal to about 20° C., for example, ranging from about −85 to about −55° C., such as a 2-ethylhexyl acrylate homopolymer, and
an intermediate block, such as an isobornyl acrylate/isobutyl methacrylate/2-ethylhexyl acrylate random copolymer.

According to additional exemplary embodiments, the block polymer comprises a first block having a glass transition temperature (Tg) of between about 20 and about 40° C., in accordance with the blocks described in c), and a second block having a glass transition temperature of less than or equal to about 20° C., as described above in b) or a glass transition temperature of greater than or equal to about 40° C., as described above.

In particular embodiments, the proportion of the first block having a Tg of between about 20 and about 40° C. ranges from about 10% to about 85%, such as from about 30% to about 80% or from about 50% to about 70% by weight of the polymer.

In embodiments in which the second block is a block having a Tg of greater than or equal to about 40° C., the second block may be present in a proportion ranging from about 10% to about 85% by weight, such as from about 20% to about 70% or from about 30% to about 70% by weight of the polymer.

In embodiments in which the second block is a block having a Tg of less than or equal to about 20° C., the second block may be present in a proportion ranging from about 10% to about 85% by weight, such as from about 20% to about 70% or from about 20% to about 50% by weight of the polymer.

In particular embodiments, the first block having a Tg of between about 20 and about 40° C. is a copolymer derived from monomers that can form homopolymers having a Tg of greater than or equal to about 40° C., and from monomers that can form homopolymers having a Tg of less than or equal to about 20° C.

In embodiments, the second block having a Tg of less than or equal to about 20° C. or having a Tg of greater than or equal to about 40° C. may be a homopolymer.

According to exemplary embodiments, the block polymer may comprise:
a first block having a Tg of between about 20 and about 40° C., such as a Tg of between about 21 to about 39° C., such as a copolymer comprising isobornyl acrylate/isobutyl methacrylate/2-ethylhexyl acrylate,
a second block having a Tg of less than or equal to about 20° C., for example ranging from about −65 to about −35° C., such as a homopolymer of methyl methacrylate, and
an intermediate block, such as an isobornyl acrylate/isobutyl methacrylate/2-ethylhexyl acrylate random copolymer.

According to additional exemplary embodiments, the block polymer may comprise:
a first block having a Tg of greater than or equal to about 40° C., for example ranging from about 85 to about 115° C., such as an isobornyl methacrylate/isobutyl methacrylate copolymer,
a second block having a Tg of less than or equal to about 20° C., for example ranging from about −35 to about −5° C., such as an isobutyl acrylate homopolymer, and
an intermediate block, such as an isobornyl methacrylate/isobutyl methacrylate/isobutyl acrylate random copolymer.

According to still other exemplary embodiments, the block polymer may comprise:
a first block having a Tg of greater than or equal to about 40° C., for example ranging from about 60 to about 90° C., such as an isobornyl acrylate/isobutyl methacrylate copolymer,
a second block having a Tg of less than or equal to about 20° C., for example ranging from about −35 to about −5° C., such as an isobutyl acrylate homopolymer, and
an intermediate block, such as an isobornyl acrylate/isobutyl methacrylate/isobutyl acrylate random copolymer.

II. Film Former Soluble in the Liquid Fatty Phase

In exemplary embodiments, the film former may be an organic film-forming polymer that is soluble in the liquid fatty phase.

In embodiments in which the liquid fatty phase of the cosmetic composition comprises at least one oil, the film former may be a polymer that is soluble in the oil. In such embodiments, as the polymer is a fat-soluble polymer. Fat-soluble polymers may be of any chemical type and, for example, may be chosen from:
a) fat-soluble, amorphous homopolymers and copolymers of olefins, of cycloolefins, of butadiene, of isoprene, of styrene, of vinyl ethers, vinyl esters or vinyl amides, or of (meth)acrylic acid esters or amides comprising a linear, branched or cyclic C₄₋₅₀ alkyl group and which may be amorphous. In particular embodiments, fat-soluble homopolymers and copolymers are obtained from monomers selected from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof. Examples of such polymers for use in embodiments include the alkyl acrylate/cycloalkyl acrylate copolymer sold by PHOENIX CHEM. under the name GIOVAREZ AC-5099 ML, and vinylpyrrolidone (VP) copolymers, such as copolymers of C₂-C₃₀ alkenes (such as C₃ to C₂₂ alkenes), and combinations thereof, may be used. As examples of VP copolymers that may be used in embodiments, mention may be made of copolymers of VP/vinyl laurate, VP/vinyl stearate, butylated polyvinylpyrrolidone (PVP), VP/hexadecene, VP/triacontene and VP/acrylic acid/lauryl methacrylate.

Particular fat-soluble copolymers that may be used in embodiments include:
i) acrylic-silicone graft polymers containing a silicone skeleton and acrylic grafts or containing an acrylic skeleton and silicone grafts, such as the product sold under the name SA 70.5 by 3M and described in U.S. Pat. Nos. 5,725,882; 5,209,924; 4,972,037; 4,981,903; 4,981,902; 5,468,477; 5,219,560; and in EP 0 388 582;
ii) fat-soluble polymers belonging to one of the classes described above and bearing fluoro groups, in particular FOMBLIN, those described in U.S. Pat. No. 5,948,393 and the alkyl (meth)acrylate/perfluoroalkyl (meth)acrylate copolymers described in EP 0 815 836 and U.S. Pat. No. 5,849,318;
iii) polymers or copolymers resulting from the polymerization or copolymerization of an ethylenic monomer, comprising one or more ethylenic bonds, which are preferably conjugated (or diene). As polymers or copolymers resulting from the polymerization or copolymerization of an ethylenic monomer, vinyl, acrylic or methacrylic copolymers may be used in embodiments.

In embodiments, the film former may be a block copolymer comprising at least one block consisting of styrene units or styrene derivatives (for example methylstyrene, chlorostyrene or chloromethylstyrene). The copolymer comprising at least one styrene block may be a diblock or triblock copolymer, or even a multiblock copolymer, in starburst or radial form. The copolymer comprising at least one styrene block may also comprise, for example, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks. The copolymer comprising at least one block consisting of styrene units or styrene derivatives may be a diblock or triblock copolymer, and, in particular embodiments, may comprise at least one block of the polystyrene/polyisoprene or polystyrene/polybutadiene type, such as those sold or manufactured under the name “LUVITOL HSB” by BASF and those of the polystyrene/copoly(ethylene-propylene) type, or alternatively of the polystyrene/copoly(ethylene-butylene) type, such as those sold or manufactured under the brand name “KRATON” by SHELLE CHEMICAL Co. or GELLED PERMETHYL 99A by PENRECO.

Examples of film formers that may be used in embodiments include KRATON G1650 (SEBS), KRATON G1651 (SEBS), KRATON G1652 (SEBS), KRATON G1657X (SEBS), KRATON G1701X (SEP), KRATON G1702X (SEP), KRATON G1726X (SEB), KRATON D-1101 (SBS), KRATON D-1102 (SBS), KRATON D-1107 (SIS), GELLED PERMETHYL 99A-750, GELLED PERMETHYL 99A-753-58 (blend of triblock and of starburst block polymer), GELLED PERMETHYL 99A-753-59 (blend of triblock and of starburst block polymer), VERSAGEL 5970 and VERSAGEL 5960 from PENRECO (blend of triblock and of starburst polymer in isododecane).

Styrene-methacrylate copolymers, such as the polymers sold under the references OS 129880, OS 129881 and OS 84383 from LUBRIZOL (styrene-methacrylate copolymer) may also be used in embodiments.

In embodiments, the film former may be chosen from copolymers of a vinyl ester (in which the vinyl group is directly attached to the oxygen atom of the ester group and the vinyl ester includes a saturated, linear or branched hydrocarbon radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (the alkyl group of which contains from 2 to 18 carbon atoms) or an allylic or methallylic ester (containing a saturated, linear or branched hydrocarbon radical of 1 to 19 carbon atoms, linked to the carbonyl of the ester group).

These copolymers may be partially crosslinked in embodiments, using crosslinking agents, which may be either of the vinyl type or of the allylic or methallylic type, such as tetraallyloxyethane, divinylbenzene, divinyl octanedioate, divinyl dodecanedioate, and divinyl octadecanedioate.

Examples of these copolymers that may be used in embodiments include: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Fat-soluble film-forming polymers that may also be used in embodiments include fat-soluble copolymers, and fat-soluble film-forming polymers resulting from the copolymerization of vinyl esters containing from 9 to 22 carbon atoms or of alkyl acrylates or methacrylates, the alkyl radicals containing from 10 to 20 carbon atoms.

Such fat-soluble copolymers may be chosen, in embodiments, from copolymers of polyvinyl stearate, polyvinyl stearate crosslinked with divinylbenzene, with diallyl ether or with diallyl phthalate, polystearyl (meth)acrylate copolymers, polyvinyl laurate and polylauryl (meth)acrylate. Such poly(meth)acrylates may be cross-linked with ethylene glycol dimethacrylate or tetraethylene glycol dimethacrylate, in embodiments.

The fat-soluble copolymers defined above are known and described in FR-A-2 232 303; such fat-soluble copolymers, for use in embodiments, may have a weight-average molecular weight ranging from about 2000 to about 500,000 and preferably from about 4000 to about 200,000.

As examples of fat-soluble polymers that may be used in exemplary embodiments, mention may be made of polyalkylenes and C₂-C₂₀ alkene copolymers, for example polybutene.
b) amorphous and fat-soluble polycondensates, such as those that do not include groups having hydrogen donating interactions, aliphatic polyesters containing C₄₋₅₀ alkyl side chains, polyesters resulting from the condensation of fatty acid dimers, or polyesters comprising a silicone segment in the form of a block, graft or end group, as defined in FR 0 113 920, and
c) amorphous and fat-soluble polysaccharides comprising alkyl (ether or ester) side chains, for example, alkylcelluloses containing a saturated or unsaturated, linear or branched C₁ to C₈ alkyl radical, such as ethylcellulose and propylcellulose.

The film-forming polymer of embodiments may be chosen from cellulose-based polymers, such as nitrocellulose, cellulose acetate, cellulose acetobutyrate, cellulose acetopropionate or ethylcellulose; polyurethanes; acrylic polymers; vinyl polymers; polyvinyl butyrals; alkyd resins; resins derived from aldehyde condensation products, such as arylsulphonamide-formaldehyde resins, for example toluenesulphonamide-formaldehyde resin; and arylsulphonamide epoxy resins.

Film-forming polymers that may be used in embodiments include nitrocellulose RS ⅛ sec.; RS ¼ sec.; ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; SS 5 sec., sold by HERCULES; the toluenesulphonamide-formaldehyde resins “KETJENTFLEX MS80” from AKZO or “SANTOLITE MHP” and “SANTOLITE MS80” from FACONNIER or “RESIMPOL 80” from PAN AMERICANA, the alkyd resin “BECKOSOL ODE 230-70-E” from DAINIPPON, the acrylic resin “ACRYLOID B66” from ROHM & HAAS, and the polyurethane resin “TRIXENE PR 4127” from BAXENDEN.
d) silicone resins, which are generally soluble or swellable in silicone oils, including cross-linked polyorganosiloxane polymers.

In the nomenclature of silicone resins, “MDTQ” resins are described as a function of the various siloxane monomer units included in the resins, with each of the letters “MDTQ” indicating a type of unit.

The letter M represents the monofunctional unit of formula (CH₃)₃SiO_{1/2}, the silicon atom being linked to only one oxygen atom in the polymer comprising this unit.

The letter D denotes a difunctional unit (CH₃)₂SiO_{2/2} in which the silicon atom is linked to two oxygen atoms.

The letter T represents a trifunctional unit of formula (CH₃)SiO_{3/2}.

In the units M, D and T defined above, at least one of the methyl groups may be substituted with a group R other than the methyl group, such as a hydrocarbon radical (especially alkyl) containing from 2 to 10 carbon atoms or a phenyl group, or alternatively a hydroxyl group.

The letter Q represents a tetrafunctional unit SiO_{4/2} in which the silicon atom is linked to four oxygen atoms, which are linked to the rest of the polymer.

Various resins with different properties may be obtained from these various units, the properties of these polymers varying as a function of the type of monomers (or units), the type and number of substituted radicals, the length of the polymer chain, the degree of branching and the size of the pendent chains.

Examples of such silicone resins that may be used in embodiments include:
siloxysilicates, such as trimethylsiloxysilicates of formula (XXI):
   [(CH₃)₃—Si—O]ₓ—(SiO_{4/2})_{y} (XXI)
(units MQ), in which x and y are integers ranging from 50 to 80,
polysilsesquioxanes of formula (CH₃SiO_{3/2})ₓ (units T), in which x is greater than 100 and at least one of the methyl radicals of which may be substituted with a group R as defined above,
the polymethylsilsesquioxanes, such as polysilsesquioxanes in which none of the methyl radicals is substituted with another group. Such polymethylsilsesquioxanes are described in U.S. Pat. No. 5,246,694, which is entirely incorporated herein by reference.

In embodiments, commercially available polymethylsilsesquioxane resins may be included, such as those sold:
by WACKER under the reference RESIN M, such as BELSIL PMS MK: polymer comprising CH₃SiO_{3/2} repeating units (units T), which may also comprise up to 1% by weight of (CH₃)₂SiO_{2/2} units (units D) and having an average molecular weight of about 10 000,
by SHIN-ETSU under the references KR-220L, which are composed of units T of formula CH₃SiO_{3/2} and contain Si—OH (silanol) end groups, under the reference KR-242A, which comprise 98% of units T and 2% of dimethyl units D and contain Si—OH end groups, or under the reference KR-251, comprising 88% of units T and 12% of dimethyl units D and contain Si—OH end groups.

Siloxysilicate resins that may be used in embodiments include trimethyl siloxysilicate resins (TMS) optionally in the form of powders. Such resins are sold under the reference SR1000 by GENERAL ELECTRIC or under the reference TMS 803 by WACKER. Mention may also be made of trimethyl siloxysilicate resins sold in a solvent such as cyclomethicone, such as those sold under the name “KF-7312J” by SHIN-ETSU or “DC 749” and “DC 593” by DOW CORNING.
e) Silicone polyamides of the polyorganosiloxane type, such as those described in U.S. Pat. Nos. 5,874,069; 5,919,441; 6,051,216 and 5,981,680.

According to exemplary embodiments, silicone polymers may belong to the following two families:
1) polyorganosiloxanes comprising at least two groups, located in the polymer chain, that are capable of establishing hydrogen interactions; and/or
2) polyorganosiloxanes comprising at least two groups, located on grafts or branches, that are capable of establishing hydrogen interactions.

In embodiments, polymers comprising, in the polymer chain, two groups capable of establishing hydrogen interactions may be polymers comprising at least one unit corresponding to the formula (XXII): 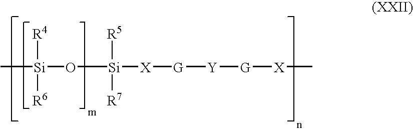
in which:
1) R⁴, R⁵, R⁶ and R⁷, which may be identical or different, are each independently a group chosen from:
   linear, branched or cyclic, saturated or unsaturated, C₁ to C₄₀ hydrocarbon groups, optionally containing one or more oxygen, sulphur and/or nitrogen atoms in the hydrocarbon chain, and optionally being partially or totally substituted with fluorine atoms,
   C₆ to C₁₀ aryl groups, optionally substituted with one or more C₁ to C₄ alkyl groups,
   polyorganosiloxane chains optionally containing one or more oxygen, sulphur and/or nitrogen atoms,
2) the groups X, which may be identical or different, may be chosen from linear or branched C₁ to C₃₀ alkylenediyl groups, optionally including one or more oxygen and/or nitrogen atoms in the chain;
3) Y is chosen from saturated or unsaturated, C₁ to C₅₀ divalent linear or branched alkylene, arylene, cycloalkylene, alkylarylene or arylalkylene groups, which optionally include comprising one or more oxygen, sulphur and/or nitrogen atoms, and/or having a substituent chosen from fluorine, hydroxyl groups, C₃ to C₈ cycloalkyl groups, C₁ to C₄₀ alkyl groups, C₅ to C₁₀ aryl groups, phenyl groups that are optionally substituted with 1 to 3 C₁ to C₃ alkyl groups, C₁ to C₃ hydroxyalkyl groups and C₁ to C₆ aminoalkyl groups; or
4) Y is a group corresponding to the formula (XXIII): 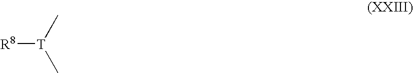
in which
   T is chosen from linear or branched, saturated or unsaturated, C₃ to C₂₄ trivalent or tetravalent hydrocarbon groups that may be optionally substituted with a polyorganosiloxane chain, and may optionally one or more atoms selected from O, N and S; and trivalent atoms selected from N, P and Al, and
   R⁸ is chosen from linear or branched C₁ to C₅₀ alkyl groups and polyorganosiloxane chains, which optionally comprise one or more ester, amide, urethane, thiocarbamate, urea, thiourea and/or sulphonamide groups, that may optionally be linked to another chain of the polymer;
5) the groups G, which may be identical or different, are divalent groups chosen from: 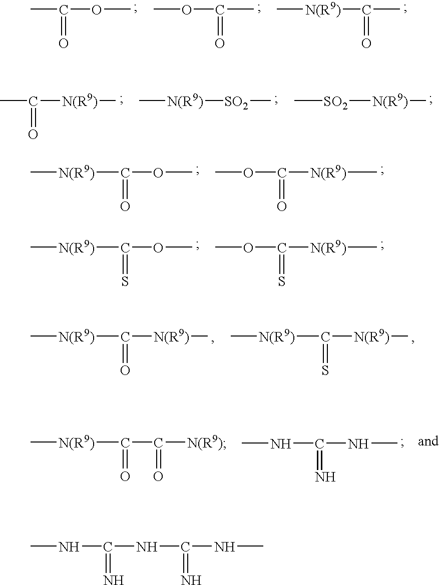
in which R⁹ is chosen from hydrogen and linear or branched C₁ to C₂₀ alkyl groups, and at least 50% of the groups R⁹ of the polymer are hydrogen atoms and at least two of the groups G of the polymer are groups other than: 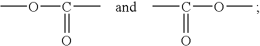
6) n is an integer in a range of from about 2 to about 500, for example from about 2 to about 200, and m is an integer in a range of from about 1 to about 1000, for example from about 1 to about 700 or from about 6 to about 200.

According to exemplary embodiments, about 80% of the groups R⁴, R⁵, R⁶ and R⁷ of the polymer may be chosen from methyl, ethyl, phenyl and 3,3,3-trifluoropropyl groups.

According to exemplary embodiments, Y can represent various divalent groups, optionally comprising one or two free valencies to establish bonds with other units of the polymer or copolymer. In some embodiments, Y represents a group chosen from:
a) linear C₁ to C₂₀ alkylene groups, for example, linear C₁ to C₁₀ alkylene groups,
b) branched C₃₀ to C₅₆ alkylene groups that optionally include rings and unconjugated unsaturations,
c) C₅-C₆ cycloalkylene groups,
d) phenylene groups that may be optionally substituted with one or more C₁ to C₄₀ alkyl groups,
e) C₁ to C₂₀ alkylene groups that include from 1 to 5 amide groups,
f) C₁ to C₂₀ alkylene groups that include one or more substituents chosen from hydroxyl, C₃ to C₈ cycloalkane, C₁ to C₃ hydroxyalkyl and C₁ to C₆ alkylamine groups,
g) polyorganosiloxane chains of formula (XXIV): 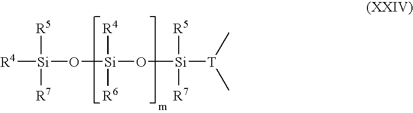
in which R⁴, R⁵, R⁶, R⁷, T and m are as defined above, and
h) polyorganosiloxane chains of formula (XXV):

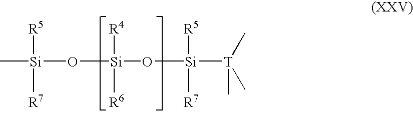

The polyorganosiloxanes of the second family, in embodiments, may be polymers that include at least one unit corresponding to formula (XXVI): 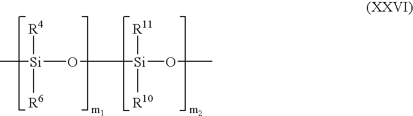
in which
   R⁴ and R⁶, which are identical or different, are as defined above for formula (XXII),
   R¹⁰ is chosen from groups as defined above for R⁴ and R⁶, or is chosen from groups of formula —X-G-R¹², in which X and G are as defined above for formula (XXII), and R¹² is chosen from hydrogen atoms, linear, branched or cyclic, saturated or unsaturated, C₁ to C₅₀ hydrocarbon groups that may optionally include one or more atoms chosen from O, S and N in the hydrocarbon chain, which may optionally be further substituted with one or more fluorine atoms and/or one or more hydroxyl groups; and phenyl groups that may be optionally substituted with one or more C₁ to C₄ alkyl groups,
   R¹¹ is chosen from groups of formula —X-G-R¹² in which X, G and R¹² are as defined above,
   m₁ is an integer ranging from 1 to 998, and
   m₂ is an integer ranging from 2 to 500.

According to exemplary embodiments, the polymer used may be a homopolymer, which includes several identical units, such as units of formula (XXII) or of formula (XXVI).

According to exemplary embodiments, polymers may be used that include copolymers including several different units of formula (XXII), such as polymers in which at least one of the groups R⁴, R⁵, R⁶, R⁷, X, G, Y, m and n is different in one of the units, and copolymers formed from several units of formula (XXVI), in which at least one of the groups R⁴, R⁶, R¹⁰, R¹¹, m₁ and m₂ is different in at least one of the units.

In embodiments, copolymers may be used that include at least one unit of formula (XXII) and at least one unit of formula (XXVI), the units of formula (XXII) and the units of formula (XXVI) possibly being identical to or different from each other.

Some embodiments may incorporate copolymers that further include at least one hydrocarbon unit having two groups capable of establishing hydrogen interactions, selected from ester, amide, sulphonamide, carbamate, thiocarbamate, urea, urethane, thiourea, oxamido, guanidino and biguanidino groups, and combinations thereof.

The copolymers of embodiments may be block copolymers or graft copolymers.

III. Film Former Insoluble in the Fatty Phase

According to exemplary embodiments, the film-forming polymer may be a solid that is insoluble in the fatty phase of the cosmetic composition at room temperature, for example at approximately 25° C. The polymer of such embodiments may also be insoluble in the fatty phase at the polymer softening point, unlike a wax, such as a polymeric wax, which is soluble in the liquid organic phase (or fatty phase) at the wax melting point. In this sense, the polymer is not a wax.

1) Polymers

The cosmetic compositions according to embodiments may comprise at least one stable dispersion of essentially spherical polymer particles of one or more polymers, in a physiologically acceptable fatty phase.

Such dispersions of particular embodiments may be in the form of polymer nanoparticles in stable dispersions in the liquid organic phase. The nanoparticles may have a mean size of between about 5 and about 800 nm, such as between about 50 and about 500 nm. However, polymer particles of some embodiments may have range in size up to about 1 μm.

In particular embodiments, the polymer particles in dispersion are insoluble in water-soluble alcohols, for instance ethanol.

The polymers in dispersion that may be used in the cosmetic compositions of embodiments may have a molecular weight of from about 2000 to about 10,000,000 g/mol and a Tg of from about −100° C. to about 300° C., such as from about −50° C. to about 100° C. or from about −10° C. to about 50° C.

In embodiments, the cosmetic compositions may include film-forming polymers having a low Tg, of less than or equal to skin temperature, for example, less than or equal to about 40° C.

Among the film-forming polymers that may be used in embodiments are acrylic or vinyl free-radical homopolymers or copolymers, such as those having a Tg of less than or equal to about 40° C., and in particular embodiments, those having a Tg ranging from about −10° C. to about 30° C., used alone or as a mixture.

As used herein, the term “free-radical polymer” encompasses polymers obtained by polymerization of unsaturated monomers, such as ethylenically unsaturated monomers, in which each monomer may homopolymerize (unlike polycondensates). The free-radical polymers of embodiments may be vinyl polymers or copolymers, for example, acrylic polymers.

The acrylic polymers of embodiments may result from the polymerization of ethylenically unsaturated monomers containing at least one acid group and/or esters of these acid monomers and/or amides of these acids.

Monomers bearing an acid group that may be used in embodiments include α,β-ethylenic unsaturated carboxylic acids such as acrylic acid, methacrylic acid, crotonic acid, maleic acid or itaconic acid. (Meth)acrylic acid and crotonic acid are used in some embodiments, and some embodiments include (meth)acrylic acid.

The acid monomer esters of embodiments may be chosen from (meth)acrylic acid esters (also known as (meth)acrylates), for instance alkyl (meth)acrylates, of C₁-C₂₀ or C₁-C₈ alkyl, aryl (meth)acrylates; of C₆-C₁₀ aryl, and hydroxyalkyl (meth)acrylates, such as of a C₂-C₆ hydroxyalkyl (meth)acrylate. Alkyl (meth)acrylates that may be used in embodiments include methyl, ethyl, butyl, isobutyl, 2-ethylhexyl and lauryl (meth)acrylate. Hydroxyalkyl (meth)acrylates that may also be included in embodiments include hydroxyethyl (meth)acrylate and 2-hydroxypropyl (meth)acrylate. Aryl (meth)acrylates that may be used in embodiments include benzyl or phenyl acrylates.

The (meth)acrylic acid esters that may be suitable for exemplary embodiments of cosmetic compositions are the alkyl (meth)acrylates.

Free-radical polymers that are used in exemplary embodiments include copolymers of (meth)acrylic acid and of alkyl (meth)acrylate, such as of a C₁-C₄ alkyl (meth)acrylate. Methyl acrylates optionally copolymerized with acrylic acid may be used for particular embodiments.

Amides of the acid monomers that may be used in embodiments include (meth)acrylamides, such as N-alkyl(meth)acrylamides, of C₂-C₁₂ alkyls, such as N-ethylacrylamide, N-t-butylacrylamide and N-octylacrylamide; N-di(C₁-C₄)alkyl(meth)acrylamides.

In embodiments, acrylic polymers may also result from the polymerization of ethylenically unsaturated monomers containing at least one amine group, in free form or in partially or totally neutralized form, or alternatively in partially or totally quaternized form. Such monomers may be, for example, dimethylaminoethyl (meth)acrylate, dimethylaminoethylmethacrylamide, vinylamine, vinylpyridine or diallyldimethylammonium chloride.

The vinyl polymers of embodiments may also result from the homopolymerization or copolymerization of at least one monomer selected from vinyl esters and styrene monomers. Such monomers may be polymerized with acid monomers and/or esters thereof and/or amides thereof, such as those mentioned previously. Examples of vinyl esters that may be mentioned for use in embodiments include vinyl acetate, vinyl propionate, vinyl neodecanoate, vinyl pivalate, vinyl benzoate and vinyl t-butylbenzoate. Styrene monomers that may be mentioned in embodiments include styrene and α-methylstyrene.

The list of monomers given is not limitative, and embodiments may use any monomer known to those skilled in the art and included in the categories of acrylic and vinyl monomers (including monomers modified with a silicone chain).

As other vinyl monomers that may be used in embodiments, mention may also be made of:
N-vinylpyrrolidone, N-vinylcaprolactam, vinyl-N-(C₁-C₆)alkylpyrroles, vinyloxazoles, vinylthiazoles, vinylpyrimidines and vinylimidazoles, and
olefins, such as ethylene, propylene, butylene, isoprene or butadiene.

The vinyl polymer of embodiments may be crosslinked with one or more difunctional monomers, for example, difunctional monomers that include at least two ethylenic unsaturations, such as ethylene glycol dimethacrylate or diallyl phthalate.

In exemplary embodiments, the polymers in dispersion may be chosen from the non-limiting groups of polymers or copolymers that includes: polyurethanes, polyurethane-acrylics, polyureas, polyurea-polyurethanes, polyester-polyurethanes, polyether-polyurethanes, polyesters, polyesteramides, alkyds; acrylic and/or vinyl polymers or copolymers; acrylic-silicone copolymers; polyacrylamides; silicone polymers, for instance silicone polyurethanes or silicone acrylics; and fluoro polymers; and mixtures thereof.

The polymer(s) in dispersion in the fatty phase of embodiments may represent from about 5% to about 40% of the weight of solids in the cosmetic composition, such as from about 5% to about 35% or from about 8% to about 30% of the weight of solids in the composition.

2) Stabilizer

According to embodiments, the polymer particles in dispersion are surface-stabilized with a stabilizer that is solid at room temperature. In such embodiments, the amount of solids in the dispersion represents the total amount of polymer and of stabilizer, and the amount of polymer present cannot be less than about 5% in these embodiments.

The polymer particles are, in particular embodiments, surface-stabilized by means of a stabilizer that may be a block polymer, a graft polymer and/or a random polymer, alone or as a mixture. The stabilization may take place by any known means, such as, for example, by direct addition of the stabilizing polymer during the polymerization.

In some embodiments, the stabilizer may also be present in the mixture before polymerization of the polymer. However, stabilizer may also be added continuously in embodiments, such as in embodiments in which the monomers are also added continuously.

In exemplary embodiments, the stabilizer may be used in amounts ranging from about 2 to about 30% by weight, such as amounts ranging from about 5 to about 20% by weight, relative to the initial monomer mixture.

In embodiments in which a graft polymer and/or a block polymer is used as stabilizer, the synthesis solvent may be chosen from solvents in which at least some of the grafts or blocks of said polymer-stabilizer are soluble, while other grafts or blocks may be insoluble therein. The polymer-stabilizer used during the polymerization of embodiments should be soluble, or dispersible, in the synthesis solvent. In some further embodiments, a stabilizer that includes insoluble blocks or grafts having an affinity for the polymer formed during the polymerization may be chosen as the stabilizer.

Among the graft polymers that may be mentioned for use in embodiments are silicone polymers grafted with a hydrocarbon chain, and hydrocarbon polymers grafted with a silicone chain.

In embodiments, the cosmetic compositions may include graft-block or block copolymers that include at least one block of polyorganosiloxane type and at least one block of a free-radical polymer, for instance graft copolymers of acrylic/silicone type. Graft copolymers of acrylic/silicone types may be used in embodiments in which the non-aqueous medium contains silicone.

In some embodiments, graft-block or block copolymers comprising at least one block of polyorganosiloxane type and at least one block of a polyether may be used. The polyorganopolysiloxane block of embodiments may be a polydimethylsiloxane or a poly(C₂-C₁₈)alkylmethylsiloxane; and the polyether block of embodiments may be a poly(C₂-C₁₈)-alkylene, such polyoxyethylene and/or polyoxypropylene. In particular embodiments, dimethicone copolyols or (C₂-C₁₈)alkyl dimethicone copolyols such as those sold under the name “DOW CORNING 3225C” by DOW CORNING, and lauryl methicones such as those sold under the name “DOW CORNING Q2-5200” by DOW CORNING, may be used.

Graft-block or block copolymers that may also be used in embodiments include those comprising at least one block resulting from the polymerization of at least one ethylenic monomer containing one or more optionally conjugated ethylenic bonds, for instance ethylene or dienes such as butadiene and isoprene, and of at least one block of a vinyl polymer, such as a styrene polymer. In embodiments in which the ethylenic monomer includes several optionally conjugated ethylenic bonds, the residual ethylenic unsaturations after the polymerization are generally hydrogenated. For example, the polymerization of isoprene leads, after hydrogenation, to the formation of an ethylene-propylene block, and the polymerization of butadiene leads, after hydrogenation, to the formation of an ethylene-butylene block. Among these polymers that may be used in embodiments are block copolymers, such as those of “diblock” or “triblock” type, for example polystyrene/polyisoprenes (SI), polystyrene/polybutadienes (SB) such as those sold under the name “LUVITOL HSB” by BASF; block copolymers of the type such as polystyrene/copoly(ethylene-propylene) (SEP) such as those sold under the name “KRATON” by SHELL CHEMICAL CO.; and block copolymers of the type such as polystyrene/copoly(ethylene-butylene) (SEB). KRATON G1650 (SEBS), KRATON G1651 (SEBS), KRATON G1652 (SEBS), KRATON G1657X (SEBS), KRATON G1701X (SEP), KRATON G1702X (SEP), KRATON G1726X (SEB), KRATON D-1101 (SBS), KRATON D-1102 (SBS) and KRATON D-1107 (SIS). These polymers are generally known as hydrogenated or non-hydrogenated diene copolymers.

GELLED PERMETHYL 99A-750, 99A-753-59 and 99A-753-58 (mixture of triblock and of star polymer), VERSAGEL 5960 from PENRECO (triblock+star polymer); OS129880, OS129881 and OS84383 from LUBRIZOL (styrene/methacrylate copolymer) may also be used in certain embodiments.

In embodiments, the graft-block or block copolymers comprising at least one block resulting from the polymerization of at least one ethylenic monomer containing one or more ethylenic bonds and of at least one block of an acrylic polymer, may be chosen from poly(methyl methacrylate)/polyisobutylene diblock or triblock copolymers and graft copolymers containing a poly(methyl methacrylate) skeleton and polyisobutylene grafts.

In embodiments, the graft-block or block copolymers comprising at least one block resulting from the polymerization of at least one ethylenic monomer containing one or more ethylenic bonds and of at least one block of a polyether such as a C₂-C₁₈ polyalkylene (especially polyethylene and/or polyoxypropylene), may be chosen from polyoxyethylene/polybutadiene or polyoxyethylene/polyisobutylene diblock and/or triblock copolymers.

In embodiments in which the stabilizer is a random polymer, the stabilizer includes a sufficient amount of groups to impart solubility in the synthesis solvent.

Copolymers based on alkyl acrylates or methacrylates derived from C₁-C₄ alcohols and on alkyl acrylates or methacrylates derived from C₈-C₃₀ alcohols may be used as the stabilizer of certain embodiments, and stearyl methacrylate/methyl methacrylate copolymers may be included in specific embodiments.

In embodiments having an apolar synthesis solvent, a polymer may be selected as a stabilizer in order to provide the fullest possible coverage of the particles, in which several polymer-stabilizer chains can be adsorbed onto a polymer particle obtained by polymerization.

In such embodiments, the stabilizer may be either a graft polymer or a block polymer, so as to have better interfacial activity. For example, blocks or grafts that are insoluble in the synthesis solvent provide bulkier coverage at the surface of the particles.

In embodiments in which the synthesis solvent includes at least one silicone oil, the stabilizer may be chosen from the group consisting of graft-block or block copolymers including at least one block of polyorganosiloxane type and at least one block of a free-radical polymer and graft-block or block copolymers of a polyether or of a polyester, for instance polyoxypropylenated and/or oxyethylenated blocks.

In embodiments in which the synthesis solvent does not include silicone oils, the stabilizer may be chosen from the group consisting of graft-block or block copolymers comprising at least one block of polyorganosiloxane type and at least one block of a free-radical polymer or of a polyether or a polyester, copolymers of alkyl acrylates or methacrylates derived from C₁-C₄ alcohols and of alkyl acrylates or methacrylates derived from C₈-C₃₀ alcohols, graft-block or block copolymers comprising at least one block resulting from the polymerization of at least one ethylenic monomer containing conjugated ethylenic bonds, and at least one block of a vinyl or acrylic polymer or of a polyether or of a polyester, or mixtures thereof.

Diblock polymers may be used as stabilizers in some embodiments.

In exemplary embodiments, the film-forming polymer that is fat-soluble or is in dispersion in a fatty phase may also be used in an amount ranging from about 0.01% to about 20% (as active material), for instance from about 1% to about 10%, where appropriate, relative to the total weight of the composition.

IV. Film Former Dispersible in the Aqueous Phase

According to other exemplary embodiments, in which the cosmetic composition includes an aqueous phase, the film-forming polymer may be chosen from aqueous dispersions of polymer particles.

The aqueous dispersion comprising one or more film-forming polymers, in embodiments, may be prepared by a person skilled in the art on the basis of his or her general knowledge, and, in particular embodiments, may be prepared by emulsion polymerization or by dispersion of the preformed polymer.

Among the film-forming polymers that may be used in the composition according to exemplary embodiments, mention may be made of synthetic polymers, of polycondensate type or of free-radical type, polymers of natural origin and mixtures thereof.

1) Polycondensates

Among the polycondensates that may be used as film-forming polymers in embodiments, mention may be made of anionic, cationic, nonionic or amphoteric polyurethanes, polyurethane-acrylics, polyurethane-polyvinylpyrrolidones, polyester-polyurethanes, polyether-polyurethanes, polyureas, polyurea/polyurethanes, and mixtures thereof.

The polyurethanes of embodiments may be, for example, an aliphatic, cycloaliphatic or aromatic polyurethane, polyurea/polyurethane or polyurea copolymer, containing, alone or as a mixture:
at least one block of linear or branched aliphatic and/or cycloaliphatic and/or aromatic polyester origin, and/or
at least one block of aliphatic and/or cycloaliphatic and/or aromatic polyether origin, and/or
at least one substituted or unsubstituted, branched or unbranched silicone block, for example polydimethylsiloxane or polymethylphenylsiloxane, and/or
at least one block comprising fluoro groups.

The polyurethanes used in embodiments may be obtained from branched or unbranched polyesters or from alkyds containing mobile hydrogens, which are modified by means of a polyaddition with a diisocyanate and a difunctional organic co-reactive compound (for example dihydro, diamino or hydroxyamino), also containing either a carboxylic acid or carboxylate group, or a sulphonic acid or sulphonate group, or alternatively a neutralizable tertiary amine group or a quaternary ammonium group.

In some embodiments, the film-forming polymer may be chosen from polyesters, polyesteramides, fatty-chain polyesters, polyamides and epoxy ester resins.

The polyesters of embodiments may be obtained, in a known manner, by polycondensation of aliphatic or aromatic diacids with aliphatic or aromatic diols or with polyols. Succinic acid, glutaric acid, adipic acid, pimelic acid, suberic acid or sebacic acid may be used as aliphatic diacids. Terephthalic acid or isophthalic acid, or alternatively a derivative such as phthalic anhydride, may be used as aromatic diacids. Ethylene glycol, propylene glycol, diethylene glycol, neopentyl glycol, cyclohexanedimethanol and 4,4-N-(1-methylpropylidene)bisphenol may be used as aliphatic diols. Glycerol, pentaerythritol, sorbitol and trimethylolpropane may be used as polyols.

The polyesteramides of embodiments may be obtained in a similar manner to the polyesters, by polycondensation of diacids with diamines or amino alcohols. Ethylenediamine, hexamethylenediamine or meta- or para-phenylenediamine may be used as diamine. Monoethanolamine may be used as amino alcohol.

In embodiments, monomer bearing an anionic group that may be used during the polycondensation may be chosen from, for example, dimethylolpropionic acid, trimellitic acid or a derivative such as trimellitic anhydride, the sodium salt of pentanediol-3-sulphonic acid and the sodium salt of 5-sulpho-1,3-benzenedicarboxylic acid. The fatty-chain polyesters may be obtained using fatty-chain diols during the polycondensation. The epoxy ester resins may be obtained by polycondensation of fatty acids with a condensate at the α,ω-diepoxy ends.

The free-radical polymers may, in particular embodiments, be acrylic and/or vinyl polymers or copolymers. Anionic radical polymers may be included in some embodiments. In exemplary embodiments, monomer bearing an anionic group that may be used during the free-radical polymerization may be chosen from acrylic acid, methacrylic acid, crotonic acid, maleic anhydride or 2-acrylamido-2-methylpropanesulphonic acid.

The acrylic polymers of embodiments may result from the copolymerization of monomers chosen from the esters and/or amides of acrylic acid or of methacrylic acid. As examples of monomers of ester type, mention may be made of methyl methacrylate, ethyl methacrylate, butyl methacrylate, isobutyl methacrylate, 2-ethylhexyl methacrylate and lauryl methacrylate. As examples of monomers of amide type, mention may be made of N-t-butylacrylamide and N-t-octylacrylamide.

Acrylic polymers obtained by copolymerization of ethylenically unsaturated monomers containing hydrophilic groups, including nonionic groups, such as hydroxyethyl acrylate, 2-hydroxypropyl acrylate, hydroxyethyl methacrylate and 2-hydroxypropyl methacrylate, are used in particular embodiments.

In some embodiments, vinyl polymers may result from the homopolymerization or copolymerization of monomers chosen from vinyl esters, styrene or butadiene. As examples of vinyl esters, mention may be made of vinyl acetate, vinyl neodecanoate, vinyl pivalate, vinyl benzoate and vinyl t-butylbenzoate.

Acrylic/silicone copolymers or even nitrocellulose/acrylic copolymers may be used in particular embodiments.

2) Free-Radical Polymer

In exemplary embodiments, polymers may be included that result from the free-radical polymerization of one or more free-radical monomers inside and/or partially at the surface of preexisting particles of at least one polymer chosen from the group consisting of polyurethanes, polyureas, polyesters, polyesteramides and/or alkyds. These polymers are generally referred to as “hybrid polymers”.

In embodiments including an aqueous dispersion of polymer particles, the solids content of the aqueous dispersion may be from about 3% to about 60%, such as from about 10% to about 50%, by weight.

The size of the polymer particles in aqueous dispersion in embodiments may be between about 10 and about 500 nm, for example, between about 20 and about 150 nm, allowing the production of a film of noteworthy gloss. However, particle sizes ranging up to about 1 micron may be used.

Aqueous dispersions of film-forming polymers that may be used in embodiments include the acrylic dispersions sold under the names “NEOCRYL XK-90®”, “NEOCRYL A-1070®”, “NEOCRYL A-1090®”, “NEOCRYL BT-62®”, “NEOCRYL A-1079®” and “NEOCRYL A-523®” by AVECIA-NEORESINS; “DOW LATEX 432®” by DOW CHEMICAL, “DAITOSOL 5000 AD®;” “DAITOSOL 5000 SJ” by DAITO KASEY KOGYO; “SYNTRAN 5760” by INTERPOLYMER; aqueous dispersions of polyurethane sold under the names “NEOREZ R-981®” and “NEOREZ R-974®” by AVECIA-NEORESINS; “AVALURE UR-405®”, “AVALURE UR-410®”, “AVALURE UR-425®”, “AVALURE UR-450®”, “SANCURE 875®”, “SANCURE 861®”, “SANCURE 878®” and “SANCURE 2060®” by GOODRICH; “IMPRANIL 85®” by BAYER and “AQUAMERE H-1511®” by HYDROMER; the sulphopolyesters sold under the brand name “EASTMAN AQ®” by EASTMAN CHEMICAL PRODUCTS; vinyl dispersions, for instance MEXOMER PAM; aqueous dispersions of polyvinyl acetate, for instance “VINYBRAN®” from NISSHIN CHEMICAL, or those sold by the company UNION CARBIDE; aqueous dispersions of terpolymer of vinylpyrrolidone, dimethylaminopropylmethacrylamide and lauryldimethylpropylmethacrylamidoammonium chloride, such as STYLEZE W from ISP; aqueous dispersions of polyurethane/polyacrylic hybrid polymers, such as those sold under the references “HYBRIDUR®” by AIR PRODUCTS or “DUROMER®” from NATIONAL STARCH; dispersions of core/shell type, for example, those sold by ATOFINA under the reference KYNAR (core: fluoro-shell: acrylic) or those described in U.S. Pat. No. 5,188,899 (core: silica-shell: silicone); and mixtures thereof.

In embodiments in which the composition comprises an aqueous phase, the film-forming polymer may be a water-soluble polymer. The water-soluble polymer may be dissolved in the aqueous phase of the composition.

Among the water-soluble film-forming polymers that may be included in embodiments are the following cationic polymers:
1) acrylic polymers or copolymers, such as polyacrylates or polymethacrylates, which may also contain one or more units derived from comonomers that may be selected from the family of acrylamides, methacrylamides, diacetoneacrylamides, acrylamides and methacrylamides substituted on the nitrogen with lower alkyls, acrylic or methacrylic acids or esters thereof, vinyllactams such as vinylpyrrolidone or vinylcaprolactam, or vinyl esters.

As copolymers of the family (1), particular embodiments may include:
copolymers of acrylamide and of dimethylaminoethyl methacrylate, quaternized with dimethyl sulphate or with a dimethyl halide, such as the product sold under the name HERCOFLOC by HERCULES,
copolymers of acrylamide and of methacryloyloxyethyltrimethylammonium chloride described, for example, in EP-A-080 976 and sold under the name BINA QUAT P 100 by CIBA GEIGY,
copolymers of acrylamide and of methacryloyloxyethyltrimethylammonium methosulphate sold under the name RETEN by HERCULES,
quaternized or non-quaternized copolymers of vinylpyrrolidone/dialkylaminoalkyl acrylate or methacrylate, such as the products sold under the name “GAFQUAT” by ISP, for instance “GAFQUAT 734” or “GAFQUAT 755”, and the products denoted as “COPOLYMER 845, 958 and 937,” these polymers are described in detail in French patents 2 077 143 and 2 393 573,
terpolymers of dimethylaminoethyl methacrylate/vinylcaprolactam/vinylpyrrolidone, such as the product sold under the name GAFFIX VC 713 by ISP, and
quaternized copolymers of vinylpyrrolidone/-dimethylaminopropylmethacrylamide, such as the product sold under the name “GAFQUAT HS 100” by ISP.
2) quaternized polysaccharides, such as those described in U.S. Pat. Nos. 3,589,578 and 4,031,307, and including guar gums containing trialkylammonium cationic groups. Such products are sold in particular under the trade names JAGUAR C13S, JAGUAR C15 and JAGUAR C17 by MEYHALL.
3) quaternary copolymers of vinylpyrrolidone and of vinylimidazole;
4) chitosans or salts thereof;
5) cationic cellulose derivatives, such as copolymers of cellulose or of cellulose derivatives grafted with a water-soluble monomer including a quaternary ammonium, and described in U.S. Pat. No. 4,131,576, including, for example, hydroalkylcelluloses, for instance hydroxymethyl-, hydroxyethyl- or hydroxypropylcelluloses grafted in particular with a methacryloyloxyethyltrimethylammonium, methacrylamidopropyltrimethylammonium or dimethyldiallylammonium salt. Such cationic cellulose derivatives include the products sold under the name “CELQUAT L 200” and “CELQUAT H 100” by the NATIONAL STARCH COMPANY.

Among the film-forming water-soluble polymers that may be included in embodiments are the following amphoteric polymers:
1) polymers resulting from the copolymerization of (1) a monomer derived from a vinyl compound bearing a carboxylic group such as, acrylic acid, methacrylic acid, maleic acid, α-chloroacrylic acid, and (2) a basic monomer derived from a substituted vinyl compound containing at least one basic atom, such as, a dialkylaminoalkyl methacrylate and acrylate, and (3) a dialkylaminoalkylmethacrylamide and -acrylamide. Such compounds are described in U.S. Pat. No. 3,836,537,
2) polymers comprising units derived from:
a) at least one monomer chosen from acrylamides and methacrylamides substituted on the nitrogen with an alkyl radical,
b) at least one acidic comonomer containing one or more reactive carboxylic groups, and
c) at least one basic comonomer, such as esters containing primary, secondary, tertiary and quaternary amine substituents of acrylic and methacrylic acids, and the products of quaternization of dimethylaminoethyl methacrylate with dimethyl or diethyl sulphate,
d) crosslinked alkylpolyaminoamides totally or partially derived from polyaminoamides,
3) polymers comprising zwitterionic units,
4) chitosan-derived polymers,
5) polymers derived from the N-carboxyalkylation of chitosan, such as N-carboxymethylchitosan or N-carboxybutylchitosan sold under the name “EVALSAN” by JAN DEKKER,
6) (C₁-C₅)alkyl vinyl ether/maleic anhydride copolymers, partially modified by a semi-amidation with an N,N-dialkylaminoalkylamine, such as N,N-dimethylaminopropylamine or by a semi-esterification with an N,N-dialkanolamine. These copolymers may also comprise other vinyl comonomers, such as vinylcaprolactam.

The water-soluble film-forming polymers of embodiments may be chosen from the group consisting of:
proteins, for instance proteins of plant origin such as wheat proteins and soybean proteins, proteins of animal origin such as keratin, for example keratin hydrolysates and sulphonic keratins;
anionic, cationic, amphoteric or nonionic chitin or chitosan polymers;
cellulosic polymers, such as hydroxyethylcellulose, hydroxypropylcellulose, methylcellulose, ethylhydroxyethylcellulose and carboxymethylcellulose, and quaternized cellulose derivatives;
acrylic polymers or copolymers, such as polyacrylates or polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate;
copolymers of vinylpyrrolidone and of caprolactam; polyvinyl alcohols;
polymers of natural origin, which are optionally modified, such as:
gum arabic, guar gum, xanthan derivatives, karaya gum;
alginates and carrageenans;
glycosaminoglycans, hyaluronic acid and derivatives thereof;
shellac resin, sandarac gum, dammar resins, elemi gums and copal resins;
deoxyribonucleic acid;
mucopolysaccharides such as hyaluronic acid and chondroitin sulphate, and mixtures thereof.

These polymers will be used in particular embodiments in which an appreciable removal of the film by water is desired.

In order to improve the film-forming nature of an oily or aqueous polymer, a coalescer, chosen from known coalescers, may be added to the polymer system of embodiments.

V. Silicone Film Former

1) Skeleton Polymer

According to exemplary embodiments, the film-forming polymer may be chosen from polymers with a non-silicone organic skeleton grafted with monomers containing a polysiloxane. These polymers may be fat-soluble, fat-dispersible, water-soluble or dispersible in aqueous medium, where appropriate.

In embodiments, such polymers containing a non-silicone organic skeleton grafted with monomers containing a polysiloxane consist of an organic main chain formed from organic monomers not comprising silicone, onto which is grafted, within said chain and also optionally on at least one of its ends, at least one polysiloxane macromer.

As used herein, and in accordance with generally accepted usage, the expression “polysiloxane macromer” encompasses any monomer containing a polysiloxane-type polymer chain in its structure.

In embodiments, the non-silicone organic monomers constituting the main chain of the graft silicone polymer can be selected from free-radical-polymerizable monomers containing ethylenic unsaturation, polycondensation-polymerizable monomers, such as those forming polyamides, polyesters or polyurethanes, and ring-opening monomers, such as those of the oxazoline or caprolactone type.

The polymers containing a non-silicone organic skeleton grafted with monomers containing a polysiloxane, in accordance with embodiments, can be obtained according to any means known to those skilled in the art, such as by reaction between (i) a starting polysiloxane macromer that is functionalized on the polysiloxane chain and (ii) one or more non-silicone organic compounds, that are functionalized with a function capable of reacting with the functional group(s) borne by the silicone, forming a covalent bond. A classic example of such a reaction is the free-radical reaction between a vinyl group borne on one of the ends of the silicone with a double bond of a monomer containing ethylenic unsaturation in the main chain.

The polymers containing a non-silicone organic skeleton grafted with monomers containing a polysiloxane, in accordance with embodiments, may be chosen from those described in U.S. Pat. Nos. 4,693,935; 4,728,571 and 4,972,037 and in patent applications EP-A-0 412 704, EP-A-0 412 707 and EP-A-0 640 105, and international patent application publication WO 95/00578. These are copolymers obtained by free-radical polymerization starting with monomers containing ethylenic unsaturation and monomers having a vinyl end group, or alternatively copolymers obtained by reaction of a polyolefin comprising functionalized groups and a polysiloxane macromer having a terminal function which is reactive with said functionalized groups.

One particular family of graft silicone polymers that is suitable for some embodiments consists of graft silicone polymers comprising:
a) from about 0 to about 98% by weight of at least one free-radical-polymerizable lipophilic monomer (A) of low lipophilic polarity containing ethylenic unsaturation;
b) from about 0 to about 98% by weight of at least one polar hydrophilic monomer (B) containing ethylenic unsaturation, which is copolymerizable with the monomer(s) of the type (A);
c) from about 0.01% to about 50% by weight of at least one polysiloxane macromer (C) of general formula (XXVII):
   X(Y)ₙSi(R)₃₋ₘZₘ (XXVII)
in which:
   X denotes a vinyl group that is copolymerizable with the monomers (A) and (B);
   Y denotes a group having a divalent bond;
   R denotes hydrogen, C₁-C₆ alkyl or alkoxy, or C₆-C₁₂ aryl;
   Z denotes a monovalent polysiloxane unit with a number-average molecular weight of at least 500;
   n is 0 or 1 and m is an integer ranging from 1 to 3; the percentages being calculated relative to the total weight of the monomers (A), (B) and (C).

In embodiments, such polymers have a number-average molecular weight ranging from about 10,000 to about 2,000,000 and may also have a glass transition temperature Tg or a crystalline melting temperature Tm of at least about −20° C.

In embodiments, lipophilic monomers (A) may be chosen from acrylic or methacrylic acid esters of C₁-C₁₈ alcohols; methacrylic acid esters of C₁₂-C₃₀ alcohols, styrene; polystyrene macromers; vinyl acetate; vinyl propionate; α-methylstyrene; tert-butylstyrene; butadiene; cyclohexadiene; ethylene; propylene; vinyltoluene; acrylic or methacrylic acid esters of 1,1-dihydroperfluoroalkanols or of homologues thereof; acrylic or methacrylic acid esters of ω-hydrofluoroalkanols; acrylic or methacrylic acid esters of fluoroalkylsulphonamido alcohols; acrylic or methacrylic acid esters of fluoroalkyl alcohols; acrylic or methacrylic acid esters of fluoroether alcohols; or mixtures thereof. In particular embodiments, monomers (A) are chosen from the group consisting of n-butyl methacrylate, isobutyl methacrylate, tert-butyl acrylate, tert-butyl methacrylate, 2-ethylhexyl methacrylate, methyl methacrylate, 2-(N-methylperfluorooctanesulphonamido)ethyl acrylate and 2-(N-butylperfluorooctanesulphonamido)ethyl acrylate, or mixtures thereof.

In embodiments, polar monomers (B) may be chosen from acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and hemiesters thereof, hydroxyalkyl (meth)acrylates, diallyldimethylammonium chloride, vinylpyrrolidone, vinyl ethers, maleimides, vinylpyridine, vinylimidazole, heterocyclic vinyl polar compounds, styrene sulphonate, allyl alcohol, vinyl alcohol and vinylcaprolactam, or mixtures thereof. In some embodiments, monomers (B) are chosen from the group consisting of acrylic acid, N,N-dimethylacrylamide, dimethylaminoethyl methacrylate, quaternized dimethylaminoethyl methacrylate and vinylpyrrolidone, and mixtures thereof.

In particular embodiments, monomer (A) may be chosen from the product KP 561 or KP 562 sold by SHIN-ETSU from esters of a C₁₈-C₂₂ alcohol and of methacrylic acid.

The polysiloxane macromers (C) of formula (XXVII) may be, in embodiments, chosen from macromers corresponding to the general formula (XXVIII) below: 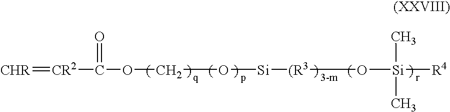
in which:
   R¹ is hydrogen or —COOH;
   R² is hydrogen, methyl or —CH₂COOH;
   R³ is C₁-C₆ alkyl, alkoxy, or alkylamino, C₆-C₁₂ aryl or hydroxyl;
   R⁴ is C₁-C₆ alkyl, alkoxy or alkylamino, C₆-C₁₂ aryl or hydroxyl;
   q is an integer ranging from 2 to 6, and in particular embodiments, q is 3;
   p is 0 or 1;
   r is an integer ranging from 5 to 700;
   m is an integer ranging from 1 to 3, and in particular embodiments, m is 1.

In embodiments, macromer (C) may be chosen from polysiloxane macromers of formula (XXIX): 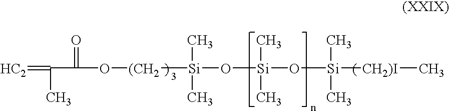
in which n ranges ranging from 5 to 700 and I is an integer between 0 and 3.

In particular embodiments, the copolymer may be obtained by free-radical polymerization of a monomer mixture consisting of:
a) about 60% by weight of tert-butyl acrylate;
b) about 20% by weight of acrylic acid;
c) about 20% by weight of silicone macromer of formula (XXX): 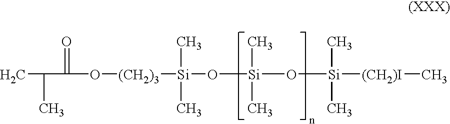
in which n ranges from 5 to 700, I is an integer between 0 and 3, and the weight percentages are calculated relative to the total weight of the monomers.

Particular embodiments of the invention also may include copolymers obtained by free-radical polymerization of a monomer mixture consisting of:
a) about 80% by weight of tert-butyl acrylate;
b) about 20% by weight of silicone macromer of formula (XXXI): 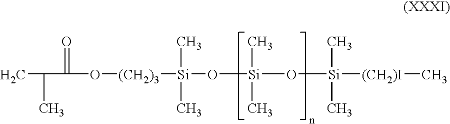
in which n ranges from 5 to 700, I is integer between 0 and 3, and the weight percentages are calculated relative to the total weight of the monomers.

Another family of graft silicone polymers with a non-silicone organic skeleton that may be used in exemplary embodiments includes graft silicone copolymers that may be obtained by reactive extrusion-molding of a polysiloxane macromer with a reactive terminal function on a polyolefin-type polymer that includes reactive groups capable of reacting with the terminal function of the polysiloxane macromer to form a covalent bond for grafting the silicone onto the main polyolefin chain. These polymers are described, along with a process for their preparation, in WO 95/00578.

The reactive polyolefins for use in embodiments may be chosen from polyethylenes and polymers of ethylene-derived monomers such as propylene, styrene, alkylstyrene, butylene, butadiene, (meth)acrylates, vinyl esters or equivalents, comprising reactive functions capable of reacting with the terminal function of the polysiloxane macromer. Reactive polyolefins of particular embodiments may be chosen from copolymers of ethylene or of ethylene derivatives and of monomers having a carboxylic function such as (meth)acrylic acid; copolymers of ethylene or of ethylene derivatives and of monomers having an acid anhydride function such as maleic anhydride; copolymers of ethylene or of ethylene derivatives and of monomers having an acid chloride function such as (meth)acryloyl chloride; copolymers of ethylene or of ethylene derivatives and of monomers having an ester function such as (meth)acrylic acid esters; and copolymers of ethylene or of ethylene derivatives and of monomers having an isocyanate function.

The silicone macromers of exemplary embodiments may be chosen from polysiloxanes including a functionalized group, at the end of the polysiloxane chain or close to the end of said chain, chosen from alcohols, thiols, epoxy groups and primary and secondary amines, and from silicone macromers of general formula (XXXII):
T-(CH₂)₆—Si—[—(OSiR⁵R⁶)ₜ—R⁷]_{y} (XXXII)
in which T is chosen from the group consisting of NH₂, NHRN, epoxy, OH, or SH function; R⁵, R⁶, R⁷ and RN are independently chosen from C₁-C₆ alkyl, phenyl, benzyl, C₆-C₁₂ alkylphenyl and hydrogen; s ranges from 2 to 100; t ranges from 0 to 1000 and y ranges from 1 to 3. These silicone macromers may have a number-average molecular weight ranging from 5000 to 300,000, such as from 8000 to 200,000 or from 9000 to 40,000.

According to particular embodiments, the film-forming polymer may be purchased from the MINNESOTA MINING AND MANUFACTURING COMPANY under the trade name “SILICONE PLUS” polymers. For example, poly(isobutyl methacrylate-co-methyl FOSEA)-g-poly(dimethylsiloxane) is sold under the trade name SA 70-5 IBMMF.

2) Silicone Skeleton Polymer

In exemplary embodiments, the film-forming polymer may be chosen from silicone polymers grafted with non-silicone organic monomers. Such silicone polymers may be fat-soluble, fat-dispersible, water-soluble or dispersible in aqueous medium.

In embodiments, graft silicone polymer(s) containing a polysiloxane skeleton grafted with non-silicone organic monomers including silicone (or polysiloxane (/SiO—)ₙ) main chains having at least one organic group that does not include silicone may be grafted within the main chain and optionally on at least one end.

The polymers containing a polysiloxane skeleton grafted with non-silicone organic monomers, according to embodiments, may be existing commercial products or alternatively can be obtained by any means known to those skilled in the art, such as by reaction between (i) a starting silicone that is functionalized on one or more of these silicon atoms, and (ii) a non-silicone organic compound that is functionalized to be capable of reacting with the functional group(s) borne by the silicone, forming a covalent bond. Classic examples of such reactions are the hydrosilylation reaction between /Si—H groups and vinyl groups CH₂═CH—, and the reaction between thio functional groups —SH with vinyl groups.

Examples of polymers containing a polysiloxane skeleton grafted with non-silicone organic monomers that are suitable for use in embodiments, are described in EP-A-0 582 152, WO 93/23009 and WO 95/03776, the teachings of which are incorporated in their entirety herein. These references also describe the preparation of these polymers.

According to some embodiments, the silicone polymer containing a polysiloxane skeleton grafted with non-silicone organic monomers includes the result of a free-radical copolymerization between (1) at least one non-silicone anionic organic monomer containing ethylenic unsaturation and/or a non-silicone hydrophobic organic monomer containing ethylenic unsaturation, and (2) a silicone containing in its chain at least one, and in certain embodiments two or more, functional group(s), such as thio functional groups, that are capable of reacting with said ethylenic unsaturations of said non-silicone monomers, to form a covalent bond.

According to embodiments, anionic monomers containing ethylenic unsaturation may be chosen, alone or as mixtures, from linear or branched, unsaturated carboxylic acids, optionally partially or totally neutralized in the form of a salt, such as, acrylic acid, methacrylic acid, maleic acid, itaconic acid, fumaric acid and crotonic acid. The suitable salts may include alkali metal salts, alkaline-earth metal salts and ammonium salts. In embodiments, the anionic organic group of the final graft silicon polymer includes the result of the free-radical (homo)polymerization of at least one anionic monomer of unsaturated carboxylic acid type and can, after reaction, be post-neutralized with a base (sodium hydroxide, aqueous ammonia, etc.) to form a salt.

According to embodiments, the hydrophobic monomers containing ethylenic unsaturation may be chosen, alone or as a mixture, from acrylic acid esters of alkanols and/or methacrylic acid esters of alkanols. The alkanols may be C₁-C₃₀ alkanols, such as C₁-C₂₂ alkanols. The hydrophobic monomers of particular embodiments may be chosen from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof.

One family of silicone polymers containing a polysiloxane skeleton grafted with non-silicone organic monomers that may be used in embodiments includes silicone polymers having the unit of formula (XXXIII) in the polymer structure.

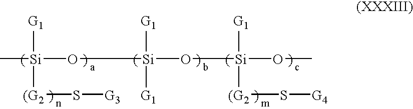

In formula (XXX), the radicals G₁, which may be identical or different, are chosen from hydrogen, C₁-C₁₀ alkyl radicals and phenyl radicals; the radicals G₂, which are identical or different, are chosen from C₁-C₁₀ alkylene groups; G₃ are chosen from polymer residues resulting from the (homo)polymerization of at least one anionic monomer containing ethylenic unsaturation; G₄ are chosen from polymer residues resulting from the (homo)polymerization of at least one hydrophobic monomer containing ethylenic unsaturation; m and n are equal to 0 or 1; a is an integer ranging from 0 to 50; b is an integer that may be between 10 and 350, c is an integer ranging from 0 to 50; and a and c is not 0.

In certain embodiments, the unit of formula (XXXIII) at least one of the following characteristics:
the radicals G₁ are chosen from alkyl radicals, such as methyl radicals;
n is not 0, and the radicals G₂ are chosen from divalent C₁-C₃ radicals, such as propylene radicals;
G₃ is chosen from polymer radicals resulting from the (homo)polymerization of at least one monomer of the carboxylic acid type containing ethylenic unsaturation, preferably acrylic acid and/or methacrylic acid; and/or
G₄ is chosen from polymer radicals resulting from the (homo)polymerization of at least one monomer of the (C₁-C₁₀) alkyl (meth)acrylate type, preferably isobutyl or methyl (meth)acrylate.

Examples of silicone polymers corresponding to formula (XXXIII) are, in embodiments, polydimethylsiloxanes (PDMSs) onto which are grafted, via a thiopropylene-type secondary bond, mixed polymer units of the poly(meth)acrylic acid type and of the polyalkyl (meth)acrylate type.

Other examples of silicone polymers corresponding to formula (XXXIII) are, in embodiments, polydimethylsiloxanes (PDMSs) onto which are grafted, via a thiopropylene-type secondary bond, polymer units of the polyisobutyl (meth)acrylate type.

Such polymers include, in some embodiments, polymers having at least one group of formula (XXXIV): 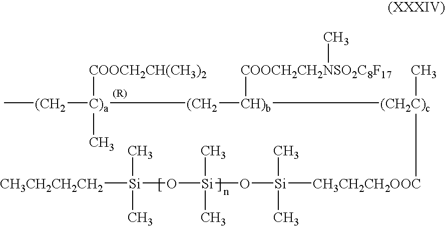
in which a, b and c, which may be identical or different, each range from 1 to 100,000; and the end groups, which may be identical or different, are each chosen from linear C₁-C₂₀ alkyl groups, branched-chain C₃-C₂₀ alkyl groups, C₃-C₂₀ aryl groups, linear C₁-C₂₀ alkoxy groups and branched C₃-C₂₀ alkoxy groups.

Such exemplary polymers are disclosed in U.S. Pat. Nos. 4,972,037; 5,061,481; 5,209,924; 5,849,275; and 6,033,650 and in international patent application publications WO 93/23446 and WO 95/06078.

Another family of silicone polymers having a polysiloxane skeleton grafted with non-silicone organic monomers, which may be used in exemplary embodiments, consists of silicone polymers including the unit of formula (XXXV) below: 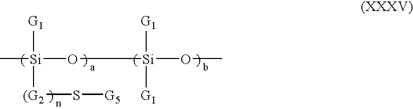
in which the radicals G₁ and G₂ have the same meaning as above; G₅ is chosen from polymer residues resulting from the (homo)polymerization of at least one ethylenically unsaturated hydrophobic monomer or from the copolymerization of at least one ethylenically unsaturated anionic monomer and of at least one ethylenically unsaturated hydrophobic monomer; n is equal to 0 or 1; a is an integer ranging from 0 to 50; b is an integer that may be between 10 and 350; and a is not 0.

In embodiments, the unit of formula (XXXV) has at least one of the following characteristics:
the radicals G₁ are chosen from alkyl radicals, such as methyl radicals; and
n is not zero, and the radicals G₂ are chosen from C₁-C₃ divalent radicals, such as radicals.

The number-average molecular mass of the silicone polymers with a polysiloxane skeleton grafted with non-silicone organic monomers of embodiments may be in a range of from about 10,000 to about 1,000,000, such as from about 10,000 to about 100,000.

According to particular embodiments, a film-forming silicone polymer may be a copolymer including carboxylate groups and polydimethylsiloxane groups.

As used herein, the expression “copolymer comprising carboxylate groups and polydimethylsiloxane groups” encompasses copolymers obtained from (a) one or more carboxylic (acid or ester) monomers, and (b) one or more polydimethylsiloxane (PDMS) chains.

As used herein, the term “carboxylic monomer” encompasses carboxylic acid monomers and carboxylic acid ester monomers. Thus, the monomer (a) may be chosen, for example, from acrylic acid, methacrylic acid, maleic acid, fumaric acid, itaconic acid, crotonic acid, esters thereof and mixtures of these monomers. Esters that may be used in particular embodiments include the following monomers: acrylate, methacrylate, maleate, fumarate, itaconate and/or crotonate. The monomers in ester form may be chosen, in embodiments, from linear or branched alkyl acrylates and methacryates, such as C₁-C₂₄ and C₁-C₂₂ alkyl acrylates and mixtures thereof. In particular embodiments, the alkyl radical may be chosen from methyl, ethyl, stearyl, butyl and 2-ethylhexyl radicals, and mixtures thereof.

The copolymer of embodiments may comprise as carboxylate groups at least one group chosen from acrylic acid and methacrylic acid, and methyl, ethyl, stearyl, butyl or 2-ethylhexyl acrylates or methacrylates, and mixtures thereof.

As used herein, and in accordance with generally accepted usage, the term “polydimethylsiloxanes” (or organopolysiloxanes and abbreviated as PDMS) encompasses any organosilicon polymer or oligomer of linear structure, of variable molecular weight, obtained by polymerization and/or polycondensation of suitably functionalized silanes, and consisting essentially of a repetition of main units in which the silicon atoms are linked together via oxygen atoms (siloxane bond ≡Si—O—Si≡), in which the linear organosilicon polymer or oligomer includes trimethyl radicals directly linked via a carbon atom to the silicon atoms. The PDMS chains that may be used to obtain the copolymer may include at least one polymerizable radical group, which may be located on at least one of the ends of the chain; that is, the PDMS may contain, for example, a polymerizable radical group on the two ends of the chain or one polymerizable radical group on one end of the chain and one trimethylsilyl end group on the other end of the chain. The polymerizable radical group may be an acrylic or methacrylic group, and in particular embodiments may be a group CH₂═CR₁—CO—O—R₂, in which R₁ is chosen from hydrogen or methyl groups and R₂ is chosen from —CH₂—, —(CH₂)ₙ— with n=3, 5, 8 or 10, —CH₂—CH(CH₃)—CH₂—, —CH₂—CH₂—O—CH₂—CH₂—, —CH₂—CH₂—O—CH₂—CH₂—CH(CH₃)—CH₂—, —CH₂—CH₂—O—CH₂—CH₂—O—CH₂—CH₂—CH₂—.

The copolymers used in embodiments are generally obtained according to usual methods of polymerization and grafting, for example by free-radical polymerization (A) of a PDMS comprising at least one polymerizable radical group (for example on one of the ends of the chain or on both ends) and (B) of at least one carboxylic monomer, as described, for example, in U.S. Pat. Nos. 5,061,481 and 5,219,560.

The copolymers of embodiments generally have a molecular weight ranging from about 3000 to about 200,000 and preferably from about 5000 to about 100,000.

The copolymer of exemplary embodiments may be in its native form or in dispersed form in a solvent, such as lower alcohols containing from 2 to 8 carbon atoms, for instance isopropyl alcohol, or oils, such as volatile silicone oils, for instance cyclopentasiloxane.

Copolymers that may be used in embodiments include, for example, of copolymers of acrylic acid and of stearyl acrylate containing polydimethylsiloxane grafts, copolymers of stearyl methacrylate containing polydimethylsiloxane grafts, copolymers of acrylic acid and of stearyl methacrylate containing polydimethylsiloxane grafts, copolymers of methyl methacrylate, butyl methacrylate, 2-ethylhexyl acrylate and stearyl methacrylate containing polydimethylsiloxane grafts. In particular embodiments, the copolymer may be chosen from the copolymers sold by SHIN-ETSU under the names KP-561 (CTFA name: acrylates/dimethicone), KP-541 in which the copolymer is dispersed at 60% by weight in isopropyl alcohol (CTFA name: acrylates/dimethicone and isopropyl alcohol), KP-545 in which the copolymer is dispersed at 30% in cyclopentasiloxane (CTFA name: acrylates/dimethicone and cyclopentasiloxane). According to some embodiments, KP561 is used and is not dispersed in a solvent, but is in waxy form, its melting point being about 30° C.

More generally, the total amount of polymer used in embodiments will be an amount sufficient to form on the skin and/or the lips a cohesive film capable of following the movements of the skin and/or the lips without becoming detached or cracking.

In embodiments in which the polymer has a glass transition temperature that is too high for the desired use, a plasticizer may be incorporated into the polymer to lower the glass temperature of the mixture used. The plasticizer may be chosen, in embodiments, from the plasticizers usually used in the field of application, and may include compounds that are able to be solvents for the polymer.

In cosmetic compositions in accordance with exemplary embodiments, the amount of film formers may vary from about 0.1% to about 20% by weight relative to the total weight of the composition, such as from about 0.5% to about 20% by weight, or from about 1% to about 20% by weight.

In exemplary embodiments of cosmetic compositions, the film former may be present in a weight ratio, relative to the weight of the silicone polymers of general formula (I), of less than about 5:1, such as less than about 1:1, or less than or equal to about 1:2.

Silicone Compounds

As used herein, the term “silicone compound” encompasses compounds, other than the silicon polymer of general formula (I), that include at least one silicon atom.

Compositions according to exemplary embodiments may include, in addition to the polymer of general formula (I), a silicone compound chosen from the aforementioned silicone oils, silicone gums, aforementioned silicone resins, silicone elastomers, and mixtures thereof.

According to particular embodiments, the compositions may include a non-volatile fluid silicone compound.

In embodiments in which the silicone compound is a silicone oil, the silicone oils may be as defined above.

In embodiments in which the silicone compound is a silicone resin, the silicone resins may be as defined above with respect to film formers.

The cosmetic compositions of embodiments may also include a silicone gum.

Silicone gums, in embodiments, are fluid or solid compounds having a weight-average molecular mass of greater than or equal to 200,000 g/mol, such as from about 200,000 to about 4,000,000 or from about 200,000 to about 2,000,000 g/mol.

The viscosity of fluid silicone gums used in embodiments may vary in the range from about 1000 to about 10,000,000 cSt, such as from about 100,000 to about 1,000,000 cSt, or from about 300,000 to about 700,000 cSt, as measured in accordance with the standard ASTM D-445.

The silicone gum of embodiments may be an ungrafted polymer, i.e. a polymer obtained by polymerizing at least one monomer without subsequent reaction of the side chains with another chemical compound. The silicone gum may be chosen, in embodiments, from dimethiconols, fluorosilicones, dimethicones and mixtures thereof. The silicone gum of particular embodiments may be a homopolymer.

In some embodiments, the silicone gum may be representable by the formula below: 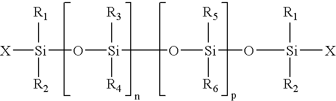
in which:
R₁, R₂, R₅ and R₆ are, independently of one another, chosen from alkyl radicals having 1 to 6 carbon atoms that are each optionally substituted by at least one fluorine atom,
R₃ and R₄ are, together or separately, chosen from alkyl radicals having 1 to 6 carbon atoms and aryl radicals,
X is chosen from alkyl radicals having 1 to 6 carbon atoms, hydroxyl radicals, vinyl or allyl radicals, and alkoxy radicals having 1 to 6 carbon atoms,
n and p are chosen to provide a silicone compound having a weight-average molecular mass of greater than or equal to 200,000 g/mol.
p may be 0 in some embodiments.

Such exemplary polymers of the above formula include dimethiconols, which are polymers in which R₁ to R₆ are methyl groups and X is a hydroxyl group. Examples include polymers of formula (IX) in which p=0 and n is between about 2000 and about 40 000, such as between about 3000 and about 30 000. Exemplary polymers may also have a molecular mass of between about 1,500,000 and about 2,000,000 g/mol.

According to embodiments, the silicone gum may be the dimethiconol sold by DOW CORNING in a polydimethylsiloxane (5 cSt) under reference D2-9085, the viscosity of the mixture being 1550 cSt, the dimethiconol sold by DOW CORNING in a polydimethylsiloxane (5 cSt) under reference DC 1503, or the dimethiconol (with a molecular weight of 1,770,000 g/mol) sold by DOW CORNING under reference Q2-1403 or Q2-1401, the viscosity of the mixture being 4000 cSt.

Silicone gums that can be used in accordance with embodiments include silicone gums in which:
substituents R₁ to R₆ and X are methyl groups, such as that sold under the name SE30 by GENERAL ELECTRIC, and that sold under the name AK 500 000 by WACKER,
substituents R₁ to R₆ and X are methyl groups and p and n are such that the molecular weight is 250,000 g/mol, such as that sold under the name SILBIONE 70047 V by RHODIA,
substituents R₁ to R₆ are methyl groups, and X is a hydroxyl group, such as that sold under the name Q2-1401 or Q2-1403 by DOW CORNING,
substituents R₁, R₂, R₅, R₆ and X are methyl groups, substituents R₃ and R₄ are aryl groups, and n and p are such that the molecular weight of the polymer is 600,000 g/mol, such as that sold under the name 761 by RHODIA-CHIMIE.

Silicone gum can be introduced into the composition according to embodiments in the form of a mixture with a silicone oil, the viscosity of said oil varying from about 0.5 to about 10,000 cSt, such as from about 0.5 to about 500 cSt, or from about 1 to about 10 cSt.

In embodiments, the silicone oil in a mixture with the silicone gum may be chosen from polyalkylsiloxanes, polyarylsiloxanes, polyalkylarylsiloxanes and mixtures thereof. The liquid silicone may be a volatile silicone such as a cyclic polydimethylsiloxane containing 3 to 7 —(CH₃)₂SiO— units.

The silicone oil of embodiments may also be a non-volatile polydimethylsiloxane silicone, such as those having a viscosity of between 8 and 10 000 cSt, for instance 10 cSt, for example the silicone sold under reference DC 200 by DOW CORNING.

The proportion of the silicone gum in the gum/oil mixture may be between about 10/90 and about 20/80 in embodiments. The viscosity of the gum/oil mixture may be between about 1000 and about 10,000 cSt in embodiments.

High molecular weight dimethicones according to embodiments include the dimethicones described in U.S. Pat. No. 4,152,416 and are sold, for example, under references SE30, SE33, SE 54 and SE 76.

Dimethicones according to embodiments may also be compounds of the above formula in which R₁ to R₆ and X are methyls and p=0. The molecular weight of such compounds may be between about 200,000 and about 300,000, such as between about 240,000 and about 260,000 g/mol.

Dimethicones according to embodiments also may include polydimethylsiloxanes, (polydimethylsiloxane)-(methylvinylsiloxane) copolymers, poly(dimethyl-siloxane)(diphenyl)(methylvinylsiloxane) copolymers, and mixtures thereof.

The fluorosilicone gums of high molecular weight according to embodiments may have molecular weights varying from about 200,000 to about 300,000 g/mol, such as from about 240,000 to about 260,000 g/mol.

The silicone compound that can be used in the cosmetic compositions of embodiments may also be a silicone elastomer. The silicone elastomer may be, in embodiments, a polyglycerolated silicone elastomer, which is different from the silicone polymer of general formula (I).

The polyglycerolated silicone elastomer that can be used in formulating the compositions according to exemplary embodiments may be an elastomeric crosslinked organopolysiloxane obtainable by crosslinking addition reaction of a diorganopolysiloxane containing at least one hydrogen bonded to the silicon and of polyglycerolated compounds having ethylenically unsaturated groups, optionally in the presence of a platinum catalyst.

In particular embodiments, the elastomeric crosslinked organopolysiloxane is obtained by crosslinking addition reaction (A) of a diorganopolysiloxane containing at least two hydrogens each bonded to a silicon, and (B) of glycerolated compounds having at least two ethylenically unsaturated groups, optionally in the presence (C) of a platinum catalyst.

In further particular embodiments, the organopolysiloxane may be obtained by reacting a polyglycerolated compound containing dimethylvinylsiloxy end groups and a methylhydropolysiloxane containing trimethylsiloxy end groups, in the presence of a platinum catalyst.

Compound (A) is the base reactant for the formation of elastomeric organopolysiloxane and the crosslinking is effected by addition reaction of the compound (A) with the compound (B) in the presence of the catalyst (C).

Compound (A) may be an organopolysiloxane having at least two hydrogen atoms bonded to different silicon atoms in each molecule.

Compound (A) may have any molecular structure, for example, a linear chain structure, a branched chain structure or a cyclic structure.

Compound (A) may have a viscosity at 25° C. ranging from 1 to 50,000 centistokes, in order to be well miscible with the compound (B), in embodiments.

The organic groups bonded to silicon atoms of the compound (A) in embodiments may be alkyl groups having 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl and 3,3,3-trifluoro-propyl; aryl groups such as phenyl, tolyl and xylyl; substituted aryl groups such as phenylethyl; and substituted monovalent hydrocarbon groups such as an epoxy group, a carboxylate ester group or a mercapto group. In particular embodiments, the organic group is chosen from methyl, phenyl and lauryl groups.

Compound (A) may thus be chosen in embodiments from methylhydropolysiloxanes containing trimethylsiloxy end groups, dimethylsiloxane-methylhydrosiloxane copolymers containing trimethylsiloxy end groups, cyclic dimethylsiloxane-methylhydrosiloxane copolymers and dimethylsiloxane-methylhydrosiloxane-laurylmethyl-siloxane copolymers containing trimethylsiloxy end groups.

Compound (B) may be a polyglycerolated compound corresponding to the formula (B′) below:
CₘH₂ₘ₋₁—O—[Gly]n-CₘH₂ₘ₋₁ (B′)
in which m is an integer ranging from 2 to 6; n is an integer ranging from 2 to 200, for example from 2 to 100, from 2 to 50, from 2 to 20, ranging from 2 to 10, or ranging from 2 to 5; and in particular embodiments, n=3; Gly denotes:
   —CH₂—CH(OH)—CH₂—O— or —CH₂—CH(CH₂OH)—O—

In exemplary embodiments, the sum of the number of ethylenic groups per molecule of compound (B) and of the number of hydrogen atoms bonded to silicon atoms per molecule of the compound (A) is at least 4.

Compound (A), in embodiments, may be added in an amount such that the molecular ratio between the total amount of hydrogen atoms bonded to silicon atoms in the compound (A) and the total amount of all of the ethylenically unsaturated groups in the compound (B) is within the range from 1/1 to 20/1.

Compound (C) is the catalyst of the crosslinking reaction of embodiments, and may be chosen from chloroplatinic acid, chloroplatinic acid-olefin complexes, chloroplatinic acid-alkenylsiloxane complexes, chloroplatinic acid-diketone complexes, platinum black, and platinum on a support.

The catalyst (C) by be added, in embodiments, in amounts of from about 0.1 to about 1000 parts by weight, such from about 1 to about 100 parts by weight, in terms of platinum metal proper, per 1000 parts by weight of the total amount of compounds (A) and (B).

The polyglycerolated silicone elastomer of embodiments may be conveyed in the form of a gel in at least one hydrocarbon oil and/or one silicone oil. In such gels the polyglycerolated elastomer may be based on non-spherical particles.

In embodiments, the polyglycerolated silicone elastomers may be chosen from those sold under the names “KSG-710”, “KSG-810”, “KSG-820”, “KSG-830”, “KSG-840” by SHIN-ETSU.

The polyglycerolated silicone elastomer may be present in the exemplary embodiments of compositions in an amount ranging from about 0.1% to about 50% by weight, relative to the total weight of the composition, such as from about 0.1% to about 40% by weight, from about 0.5% to about 30% by weight, from about 0.5% to about 20% by weight, or from about 1% to about 10% by weight.

The silicone elastomer that can be used in the cosmetic compositions in accordance with embodiments may also be chosen from emulsifying silicone elastomers.

The silicone elastomer that can be used in the cosmetic compositions in accordance with embodiments may also be chosen from non-emulsifying silicone elastomers.

As used herein, the term “non-emulsifying” defines organopolysiloxane elastomers that do not contain a hydrophilic chain, such as polyoxyalkylenated or polyglycerolated units.

The spherical non-emulsifying silicone elastomer of embodiments may be chosen from elastomeric crosslinked organopolysiloxanes that may be obtained by (1) crosslinking addition reaction of a diorganopolysiloxane containing at least one hydrogen bonded to the silicon and of a diorganopolysiloxane having ethylenically unsaturated groups bonded to the silicon, optionally in the presence of a platinum catalyst; (2) dehydrogenation crosslinking condensation reaction between a diorganopolysiloxane containing hydroxyl end groups and a diorganopolysiloxane containing at least one hydrogen bonded to the silicon, optionally in the presence of an organotin compound; (3) crosslinking condensation reaction of a diorganopolysiloxane containing hydroxyl end groups and of a hydrolysable organopolysiloxane; (4) thermal crosslinking of an organopolysiloxane, optionally in the presence of an organic peroxide catalyst; or (5) crosslinking of an organopolysiloxane by means of high-energy radiation, such as gamma rays, ultraviolet rays or electron beams.

The elastomeric crosslinked organopolysiloxane of exemplary embodiments may be obtained by crosslinking addition reaction (A2) of a diorganopolysiloxane containing at least two hydrogens each bonded to a silicon, and (B2) of a diorganopolysiloxane having at least two ethylenically unsaturated groups bonded to the silicon, optionally in the presence (C2) of a platinum catalyst, as described in EP-A-295886.

In particular embodiments, the organopolysiloxane may be obtained by reacting a dimethylpolysiloxane containing dimethylvinylsiloxy end groups and a methylhydropolysiloxane containing trimethylsiloxy end groups, in the presence of a platinum catalyst.

The compound (A2) is the base reactant for the formation of elastomeric organopolysiloxanes of embodiments, and crosslinking is effected by an addition reaction of the compound (A2) with the compound (B2) in the presence of the catalyst (C2).

The compound (A2) may be chosen, in embodiments, from diorganopolysiloxanes having at least two lower alkenyl groups (C2-C4, for example); the lower alkenyl group may be chosen from vinyl, allyl and propenyl groups. These lower alkenyl groups may be situated in any position of the organopolysiloxane molecule, but, in some embodiments, the lower alkenyl groups may be situated at the ends of the organopolysiloxane molecule. In embodiments, the organopolysiloxane (A2) may have a branched-chain, linear-chain, cyclic or network structure; and in particular embodiments, the organopolysiloxane has the linear-chain structure. The compound (A2) may have a viscosity ranging from the liquid state to the gum state, in embodiments, such as a viscosity of at least 100 centistokes at 25° C.

The organopolysiloxanes (A2) of embodiments may be chosen from methylvinylsiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxanes containing dimethylvinylsiloxy end groups, and dimethylsiloxane-methyl(3,3,3-trifluoropropyl)siloxane copolymers containing dimethylvinylsiloxy end groups.

The compound (B2) may be, in embodiments, an organopolysiloxane having at least two hydrogens bonded to the silicon in each molecule, and may act as the crosslinker of the compound (A2).

In exemplary embodiments, the sum of the number of ethylenic groups per molecule of the compound (A2) and the number of hydrogen atoms bonded to the silicon per molecule of the compound (B2) is at least 4.

The compound (B2) may be in any molecular structure, and in particular embodiments has a linear-chain, branched-chain or cyclic structure.

The compound (B2) may have a viscosity at 25° C. ranging from about 1 to about 50 000 centistokes, in embodiments, so as to be well miscible with the compound (A).

In embodiments, compound (B2) may be added in an amount such that the molecular ratio between the total amount of hydrogen atoms bonded to the silicon in the compound (B2) and the total amount of all of the ethylenically unsaturated groups in the compound (A2) is in the range from about 1/1 to about 20/1.

The compound (B2) of embodiments may be chosen from methylhydropolysiloxanes containing trimethylsiloxy end groups, dimethylsiloxane-methylhydrosiloxane copolymers containing trimethylsiloxy end groups, and cyclic dimethylsiloxane-methylhydrosiloxane copolymers.

The compound (C2) is, in embodiments, the catalyst of the crosslinking reaction, and may be chosen from chloroplatinic acid, chloroplatinic acid-olefin complexes, chloroplatinic acid-alkenylsiloxane complexes, chloroplatinic acid-diketone complexes, platinum black and platinum on a support.

The catalyst (C2) may be added at from about 0.1 to about 1000 parts by weight, such as about 1 to about 100 parts by weight, in terms of platinum metal proper, per 1000 parts by weight of the total amount of the compounds (A2) and (B2).

In exemplary embodiments, additional organic groups may be bonded to the silicon in the organopolysiloxanes (A2) and (B2) described above, and include, for example, alkyl groups such as methyl, ethyl, propyl, butyl and octyl; substituted alkyl groups such as 2-phenylethyl, 2-phenylpropyl and 3,3,3-trifluoropropyl; aryl groups such as phenyl, tolyl and xylyl; substituted aryl groups such as phenylethyl; and substituted monovalent hydrocarbon groups such as an epoxy group, a carboxylate ester group or a mercapto group.

The elastomeric crosslinked organopolysiloxane particles of embodiments may be conveyed in the form of a gel composed of an elastomeric organopolysiloxane included in at least one hydrocarbon oil and/or silicone oil. Within these gels, the organopolysiloxane particles may be non-spherical particles.

The elastomeric crosslinked organopolysiloxane particles of embodiments may also be in powder form, such as in the form of a spherical powder.

Spherical non-emulsifying elastomers are described in JP-A-61-194009, EP-A-242219, EP-A-285886, EP-A-765656, the entire content of which is incorporated herein by reference.

Spherical non-emulsifying elastomers of embodiments may include those sold under the names “DC 9040”, “DC 9041”, “DC 9509”, “DC 9505” and “DC 9506” by DOW CORNING.

In embodiments, the spherical non-emulsifying silicone elastomer may also be present in the form of an elastomeric crosslinked organopolysiloxane powder coated with silicone resin, such as with silsesquioxane resin, as described for example in U.S. Pat. No. 5,538,793, the entire content of which is incorporated herein by reference. Elastomers of this kind are sold under the names “KSP-100”, “KSP-101”, “KSP-102”, “KSP-103”, “KSP-104” and “KSP-105” by SHIN-ETSU.

Other elastomeric crosslinked organopolysiloxanes in the form of spherical powders that may be included in embodiments may be hybrid silicone powders functionalized with fluoroalkyl groups, such as those sold under the name “KSP-200” by Shin Etsu; and hybrid silicone powders functionalized with phenyl groups, such as those sold under the name “KSP-300” by Shin Etsu.

In embodiments, the non-emulsifying spherical silicone elastomer may be present in the composition in an amount ranging from about 0.1% to about 95% by weight, relative to the total weight of the composition, such as from about 0.5% to about 75% by weight, from about 1% to about 50% by weight, from about 1% to about 40% by weight, or from about 1% to about 30% by weight.

Short Hydrocarbon Ester

Exemplary embodiments of cosmetic compositions may also include a short ester, in addition to the silicon polymer and the film former.

As used herein, the term “short hydrocarbon ester” encompasses hydrocarbon esters containing less than 40 carbon atoms.

The cosmetic compositions of embodiments may include at least one short hydrocarbon ester.

The esters in accordance with embodiments may be monoesters, diesters or polyesters. In particular embodiments, the esters are monoesters, which have only one ester function. These esters may be linear, branched or cyclic and may be saturated or unsaturated. In particular embodiments, the esters may be branched and saturated. Esters of embodiments may also be volatile or non-volatile.

In particular embodiments, the hydrocarbon esters may correspond to the formula RCOOR′ in which RCOO represents a fatty acid residue containing 2 to 28 carbon atoms and R′ represents a hydrocarbon chain containing 1 to 28 carbon atoms. In some embodiments, the groups R and R′ are chosen so that the ester is non-volatile.

In exemplary embodiments, the mono-, di- or polyester hydrocarbon esters contain less than 40 carbon atoms and more than 10 carbon atoms.

These non-volatile esters of embodiments may be C₁₀ to C₂₅ or C₁₄ to C₂₂ esters. The esters may be chosen, in embodiments, from esters of C₂ to C₁₈ acids, of C₂ to C₂₀ alcohols or C₂ to C₈ polyols, and of mixtures thereof.

The esters of embodiments may contain less than 22 carbon atoms.

According to particular embodiments, the hydrocarbon ester containing less than 22 carbon atoms is not a volatile oil.

In exemplary embodiments, the esters may be chosen from a non-limitative list comprising esters of neopentanoic acid, for instance isodecyl neopentanoate, isotridecyl neopentanoate, isostearyl neopentanoate and octyldodecyl neopentanoate; esters of isononanoic acid, for instance isononyl isononanoate, octyl isononanoate, isodecyl isononanoate, isotridecyl isononanoate and isostearyl isononanoate; esters of isopropyl alcohol, such as isopropyl myristate, isopropyl palmitate, isopropyl stearate or isostearate, cetyl octanoate, tridecyl octanoate, 2-ethylhexyl 4-diheptanoate and palmitate, alkyl benzoate, polyethylene glycol diheptanoate, propylene glycol di-2-ethylhexanoate; and mixtures thereof. The esters of embodiments may also be chosen from synthetic esters, for instance synthetic esters of fatty acids, such as purcellin oil, isopropyl myristate, ethyl palmitate and octyl stearate; hydroxylated esters such as isostearyl lactate, octyl hydroxystearate, diisopropyl adipate, fatty alcohol heptanoates, octanoates and decanoates, and mixtures thereof. Particular embodiments may include esters chosen from isononyl isononanoate, isotridecyl isononanoate, stearyl heptanoate and mixtures thereof. According to particular embodiments, the short hydrocarbon ester may be a mixture of isononyl isononanoate, isotridecyl isononanoate and stearyl heptanoate.

The hydrocarbon ester(s) of embodiments may be included in a proportion of from about 5% to about 90%, such as from about 10% to about 60% or from about 20% to about 50% by weight, relative to the total weight of the composition.

Fatty Phase

The cosmetic compositions in accordance with embodiments may include a fatty phase having oils and fats that are solid at ambient temperature (20-25° C.) and atmospheric pressure.

An oil, as used herein, encompasses any fatty substance that is a liquid at ambient temperature (20-25° C.) and at atmospheric pressure. The liquid fatty phase may also contain additional compounds dissolved in the oils, such as gelling and/or structuring agents.

The cosmetic composition according to embodiments may include at least one oil(s), and particular embodiments may include at least two oils.

The oil or oils of embodiments may be present in a proportion of from about 0.1% to about 99% by weight, such as from about 1% to about 90% by weight, from about 5% to about 70% by weight, from about 10% to about 60% by weight, or from about 20% to about 50% by weight, relative to the total weight of the cosmetic composition.

The oils suitable for preparing cosmetic compositions according to embodiments may be volatile or non-volatile, silicone or non-silicone oils.

As used herein, “volatile oil” encompasses oils (or non-aqueous medium) that are capable of evaporating on contact with the skin in less than one hour at ambient temperature and atmospheric pressure. The volatile oil of embodiments may be a volatile cosmetic oil that are liquid at ambient temperature, and that may have a non-zero vapour pressure, at ambient temperature and atmospheric pressure. In particular embodiments, the volatile oil may have a vapour pressure ranging from about 0.13 Pa to about 40,000 Pa (about 10⁻³ to about 300 mmHg), such as from about 1.3 Pa to about 13,000 Pa (about 0.01 to about 100 mmHg), or from about 1.3 Pa to about 1300 Pa (about 0.01 to about 10 mmHg).

As used herein, “non-volatile oil” encompasses oils having a vapor pressure of less than about 0.13 Pa. The volatile or non-volatile oils of embodiments may be hydrocarbon oils, such as those of animal or plant origin, synthetic oils, silicone oils, fluoro oils, or mixtures thereof.

As used herein, “silicone oil” encompasses oils containing at least one silicon atom, such as those containing at least one Si—O group.

As used herein, “hydrocarbon oil” encompasses oils containing principally hydrogen and carbon atoms and optionally oxygen, nitrogen, sulphur and/or phosphorous atoms.

The volatile hydrocarbon oils of embodiments may be chosen from hydrocarbon oils having 8 to 16 carbon atoms, such as branched C₈-C₁₆ alkanes (also known as isoparaffins), for instance isododecane (also known as 2,2,4,4,6-pentamethylheptane), isodecane, isohexadecane, and, for example, the oils sold under the trade names ISOPARS® or PERMETHYLS®.

As volatile oils of embodiments, volatile silicones may be used. Such volatile silicones include, for example, volatile linear or cyclic silicones, such as those having a viscosity ≦8 centistokes (8×10⁻⁶ m²/s), and those having 2 to 10 silicon atoms, for example, silicones having 2 to 7 silicon atoms, and silicones optionally containing alkyl or alkoxy groups having 1 to 10 carbon atoms. In embodiments, volatile silicone oils may be chosen from, for example, dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

Volatile fluoro oils, such as nonafluoromethoxybutane or perfluoromethylcyclopentane, and mixtures thereof, may also be used in embodiments.

The fatty phase of the cosmetic compositions according to embodiments may also comprise at least one volatile oil.

According to particular embodiments, volatile oil may be present in amounts that are less than 30% by weight, such as less than about 15%, less than about 10%, or less than about 5%, by weight, relative to the total weight of the composition.

In other embodiments, the cosmetic compositions are free from volatile oils.

The fatty phase of the cosmetic compositions according to exemplary embodiments may also comprise at least one non-volatile oil.

The non-volatile oils of embodiments may be chosen from hydrocarbon oils, which are optionally fluorinated, and/or non-volatile silicone oils.

The non-volatile hydrocarbon oil of embodiments may be chosen from, for example:
hydrocarbon oils of animal origin;
hydrocarbon oils of plant origin, such as phytostearyl esters, for instance phytostearyl oleate, phytostearyl isostearate and lauroyl/octyldodecyl/phytostearyl glutanate (AJINOMOTO, ELDEW PS203), triglycerides consisting of fatty acid-glycerol esters, in which the fatty acids may have varied linear or branched and saturated or unsaturated chain lengths from C₄ to C₂₄; and in embodiments, the hydrocarbon oils of plant origin may be chosen from heptanoic or octanoic triglycerides, wheatgerm oil, sunflower oil, grapeseed oil, sesame oil, corn oil, apricot oil, castor oil, shea oil, avocado oil, olive oil, soya oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; karite butter; or else caprylic/capric acid triglycerides such as those sold by STEARINERIES DUBOIRS or those sold under the names MIGLYOL 810®, 812® and 818® by DYNAMIT NOBEL;
synthetic ethers having 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters like those described above;
polyol esters and pentaerythritol esters, such as dipentaerythritol tetrahydroxystearate/-tetraisostearate;
esters of diol dimers and of diacid dimers, such as the products LUSPLAN DD-DA5® and LUSPLAN DD-DA7®, sold by NIPPON FINE CHEMICAL and described in FR 0302809, the content of which is incorporated herein by reference;
fatty alcohols that are liquid at ambient temperature, containing a branched and/or unsaturated carbon chain having 12 to 26 carbon atoms, such as 2-octyldodecanol, isostearyl alcohol, oleyl alcohol, 2-hexyldecanol, 2-butyloctanol and 2-undecylpenta-decanol;
higher fatty acids, such as oleic acid, linoleic acid, linolenic acid and mixtures thereof; and
dialkyl carbonates, the two alkyl chains possibly being identical or different, such as the dicaprylyl carbonate sold under the name CETIOL CC®, by COGNIS.

The non-volatile silicone oils that can be used in compositions according to embodiments may be chosen from non-volatile polydimethylsiloxanes (PDMS), polydimethylsiloxanes containing pendent alkyl or alkoxy groups and/or alkyl or alkoxy groups that are at the ends of a silicone chain, these groups each having 2 to 24 carbon atoms, phenyl silicones, for instance phenyltrimethicones, phenyldimethicones, phenyl trimethylsiloxy diphenylsiloxanes, diphenyl dimethicones, diphenyl methyldiphenyl trisiloxanes, and 2-phenylethyl trimethylsiloxysilicates, and mixtures thereof.

In exemplary embodiments, the oil may be chosen from hydrogenated polyisobutene, isostearyl heptanoate, isononyl isononanoate, isotridecyl isononanoate, diisostearyl malate, dipentaerythritol tetrahydroxystearate/-tetraisostearate, 2-octyldodecanol, and mixtures thereof.

According to particular embodiments, the oil may be a mixture of hydrogenated polyisobutene, isostearyl heptanoate, isononyl isononanoate, isotridecyl isononanoate, diisostearyl malate, dipentaerythritol tetrahydroxystearate/-tetraisostearate and 2-octyldodecanol.

The non-volatile oils of embodiments may be present amounts ranging from about 20% to about 99% by weight, such as from about 30% to about 80% by weight or from about 40% to about 80% by weight, relative to the total weight of the composition.

According to particular embodiments, the liquid fatty phase of the cosmetic compositions is a silicone oil present in an amount ranging from about 0 to about 90% by weight, such as from about 0.1% to about 80% by weight or from about 2% to about 80% by weight, relative to the total weight of the composition.

According to embodiments, the silicone oil may be present in the cosmetic compositions in a weight ratio, relative to the silicone polymer of general formula (I), of from about 80:1, such as from about 60:1 or from about 40:1.

The liquid fatty phase of some embodiments may be thickened, gelled or structured by incorporation therein of a gelling agent for the fatty phase, as described in WO 2004/55080, which is incorporated herein by reference.

The compositions according to embodiments may also include at least one compound chosen from waxes, pasty fatty substances and mixtures thereof.

The wax of exemplary embodiments may be solid at ambient temperature (25° C.), may have a reversible solid/liquid state change, may have a melting temperature of greater than 30° C. such as up to 200° C., may have a hardness of more than 0.5 MPa, and may exhibit in the solid state an anisotropic crystalline organization. In embodiments, the wax may be hydrocarbon-, fluorine- and/or silicone-based and may be animal, vegetable, mineral or synthetic in origin. The wax of embodiments may be chosen, for example, from beeswax, carnauba wax, candelilla wax, paraffin waxes, hydrogenated castor oil, synthetic waxes such as polyethylene waxes (including those having a molecular weight of between 400 and 600) or Fischer-Tropsch waxes, silicone waxes such as alkyl- or alkoxy-dimethicones having 16 to 45 carbon atoms, ceresines or ozokerites, such as, for example, isoparaffins whose melting point is less than 40° C., such as EMW-0003, sold by NIPPON SEIROU, α-olefin oligomers, such as the PERFORMA V® polymers 825, 103 and 260, sold by NEW PHASE TECHNOLOGIES; ethylene-propylene copolymers, such as PERFORMALENE® EP 700, and microcrystalline waxes whose melting point is greater than 85° C., such as the HI-MIC® products 1070, 1080, 1090 and 3080, sold by NIPPON SEIROU, and mixtures thereof.

In exemplary embodiments, the wax is chosen from polyethylene waxes, candelilla wax and mixtures thereof.

According to particular embodiments, the cosmetic compositions include a mixture of polyethylene wax and candelilla wax.

According to particular embodiments, the wax used in the cosmetic compositions in accordance with embodiments is present in an amount varying from about 1.5% to about 20%, such as from about 3% to about 15%, from about 5% to about 10%, or from about 6.5% to about 8.5% by weight relative to the total weight of the composition.

The cosmetic compositions in accordance with embodiments may also include at least one pasty compound.

As used herein, the term “pasty” indicates fatty compounds that exhibit reversible solid/liquid state change and at a temperature of 23° C. have liquid and solid fractions. For example, polyvinyl laurate is pasty.

The pasty compound of embodiments may exhibit a hardness at 20° C. ranging from about 0.001 to about 0.5 MPa, such as from about 0.002 to about 0.4 MPa.

Pasty compounds that can be used in embodiments include, for example, lanolins and lanolin derivatives such as acetylated lanolins, oxypropylenated lanolins or isopropyl lanolate, and mixtures thereof; esters of fatty alcohols or acids, for example those having 20 to 65 carbon atoms, such as triisostearyl citrate or cetyl citrate; arachidyl propionate; polyvinyl laurate; cholesterol esters such as triglycerides of vegetable origin, for instance hydrogenated vegetable oils or hydrogenated castor oil derivatives, such as THIXINR® from Rheox; viscous polyesters; and mixtures thereof.

In some embodiments, polyesters resulting from the esterification of a carboxylic acid and an aliphatic hydroxycarboxylic acid ester may be included, such as, for example, RISOCAST® da-l (ester obtained from the esterification reaction of hydrogenated castor oil with dilinoleic acid in proportions of 2 to 1) and RISOCAST® da-h (ester resulting from the esterification of hydrogenated castor oil with isostearic acid in proportions of 4 to 3), which are sold by the Japanese company KOKYU ALCOHOL KOGYO.

Hydrogenated cocoglycerides may also be used as pasty compounds of embodiments.

In addition, the pasty compounds of embodiments may be chosen from pasty silicone compounds such as the high molecular weight polydimethylsiloxanes (PDMS), such as those having pendent chains of the alkyl or alkoxy type having 8 to 24 carbon atoms, and a melting point of 20-55° C., such as stearyldimethicones, including those sold by DOW CORNING under the trade names DC2503® and DC25514®; and mixtures thereof.

Aqueous Phase

In exemplary embodiments, cosmetic compositions may include at least one aqueous medium, constituting an aqueous phase, which may form the continuous phase of the composition.

The aqueous phase may be composed essentially of water.

The aqueous phase may be composed, in embodiments, of a mixture of water and a water-miscible organic solvent (with a miscibility in water of more than 50% by weight at 25° C.). The water-miscible organic solvent of embodiments, may be chosen from lower monoalcohols having 1 to 5 carbon atoms, for instance ethanol, isopropanol; glycols having 2 to 8 carbon atoms, for instance propylene glycol, ethylene glycol, 1,3-butylene glycol and dipropylene glycol; C₃-C₄ ketones; and C₂-C₄ aldehydes.

The aqueous phase (water and, optionally, the water-miscible organic solvent) may be present in embodiments in an amount ranging from 0.1% to 40% by weight, such as from 0.1% to 20% by weight or from 0.1% to 10% by weight, relative to the total weight of the composition.

Colorants

The cosmetic compositions of exemplary embodiments may incorporate one or more coloring agents, such as at least one colorant, organic or inorganic, which may be chosen from pigments and/or nacres conventionally used in cosmetic compositions.

As used herein, pigments encompass white or colored, mineral or organic particles that are insoluble in an aqueous solution and that color or opacify the resulting film.

The pigments of embodiments may be present in a proportion of from about 0.01% to about 15% by weight, such as from about 0.01% to about 10% by weight or from about 0.02% to about 5% by weight, relative to the total weight of the cosmetic composition. In embodiments, the pigments may be chosen from mineral pigments, such as titanium oxide, zirconium oxide or cerium oxide and also zinc oxide, iron oxide or chromium oxide, ferric blue, manganese violet, ultramarine blue and chromium hydrate.

The pigment of embodiments may be, for example, of sericite/brown iron oxide/titanium dioxide/silica structural type. Such pigments are sold for example under reference COVERLEAF NS or JS by CHEMICALS AND CATALYSTS, which has a contrast ratio of around 30.

The colorant of embodiments may also include a pigment having a structure, for example, of the type of silica microspheres containing iron oxide. An example of a pigment having this structure is that sold by MIYOSHI under reference PC BALL PC-LL-100 P, which is composed of silica microspheres containing yellow iron oxide.

The colorants of embodiments may be chosen from organic pigments, such as carbon black, D & C pigments, lakes based on cochineal carmine, on barium, strontium, calcium or aluminum, or else the diketopyrrolopyrroles (DPP) described in EP-A-542669, EP-A-787730, EP-A-787731, and WO-A-96/08537.

As used herein, “nacres” encompass colored particles of any shape, iridescent or non-iridescent, that are produced, for example, by certain mollusks in their shell or are synthesized, and that exhibit a color effect by optical interference.

The nacres of embodiments may be chosen from nacreous pigments such as titanium mica coated with an iron oxide, mica coated with bismuth oxichloride, titanium mica coated with chromium oxide, titanium mica coated with an organic dye, and nacreous pigments based on bismuth oxichloride. The nacreous pigment of embodiments may also include mica particles having at least two successive layers of metal oxides and/or of organic colorants superposed on the mica surface.

Mention may also be made, as examples of nacres for use in embodiments, of natural mica coated with titanium dioxide, with iron oxide, with natural pigment or with bismuth oxichloride.

Among nacres available on the market mention are TIMICA, FLAMENCO and DUOCHROME (based on Mica), which are sold by ENGELHARD, the TIMIRON nacres sold by MERCK, the PRESTIGE mica-based nacres sold by ECKART, and the synthetic-mica-based SUNSHINE nacres sold by SUN CHEMICAL.

The nacres of exemplary embodiments may possess a yellow, pink, red, bronze, orange, brown, gold and/or copper color or glint.

By way of illustration, nacres that may be used in embodiments include, for example, the golden nacres sold by ENGELHARD under the name Brilliant gold 212G (TIMICA), Gold 222C (Cloisonne), Sparkle gold (TIMICA), Gold 4504 (Chromalite) and Monarch gold 233X (Cloisonne); the bronze nacres sold by MERCK under the name Bronze fine (17384) (Colorona) and Bronze (17353) (Colorona) and by ENGELHARD under the name Super bronze (Cloisonne); the orange nacres sold by ENGELHARD under the name Orange 363C (Cloisonne) and Orange MCR 101 (Cosmica) and by MERCK under the name Passion orange (Colorona) and Matt orange (17449) (Microna); the brown-hued nacres sold by ENGELHARD under the name Nu-antique copper 340XB (Cloisonne) and Brown CL4509 (Chromalite); the copper-glint nacres sold by ENGELHARD under the name Copper 340A (TIMICA); the red-glint nacres sold by MERCK under the name Sienna fine (17386) (Colorona); the yellow-glint nacres sold by ENGELHARD under the name Yellow (4502) (Chromalite); the gold-glint red-hued nacres sold by ENGELHARD under the name Sunstone G012 (Gemtone); the pink nacres sold by ENGELHARD under the name Tan opal G005 (Gemtone); the gold-glint black nacres sold by ENGELHARD under the name Nu antique bronze 240 AB (TIMICA), the blue nacres sold by MERCK under the name Matt blue (17433) (Microna), the silver-glint white nacres sold by MERCK under the name XIRONA Silver, and the green-golden pinkish orangish nacres sold by MERCK under the name Indian summer (XIRONA), and mixtures thereof.

The cosmetic compositions of embodiments may also include water-soluble or fat-soluble dyes in an amount ranging from 0.01% to 10% by weight, such as from 0.01% to 5% by weight, relative to the total weight of the cosmetic composition. The fat-soluble dyes for use in embodiments may include, for example, Sudan Red, DC Red 17, DC Green 6, β-carotene, soya oil, Sudan Brown, DC Yellow 11, DC Violet 2, DC orange 5, and quinoline yellow. The water-soluble dyes of embodiments may include, for example, beetroot juice and methylene blue.

The cosmetic compositions of embodiments may also include at least one material having a specific optical effect.

The specific optical effect of embodiments is different from a simple, conventional hue effect; the specific optical effect of embodiments is a unified and stabilized effect of the kind produced by conventional colorants such as monochromatic pigments, for example. As used herein, “stabilized” signifies absence of an effect of variability of color with the angle of observation or in response to a temperature change.

For example, the material having a specific optical effect in embodiments may be chosen from particles having a metallic glint, goniochromatic coloring agents, diffracting pigments, thermochromic agents, optical brighteners, and also fibers, which may be interference type fibers. Different materials having specific optical effects may be combined in such a way as to produce the simultaneous manifestation of two effects or the manifestation of another effect in embodiments.

The metallic-glint particles of embodiments may be chosen from:
particles of at least one metal and/or of at least one metal derivative;
particles including a single-substance or multi-substance, organic or mineral substrate, at least partly coated with at least one metal-glint layer that includes at least one metal and/or at least one metal derivative; and
mixtures of said particles.

The metals that can be present in these particles of embodiments include, for example, of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and their mixtures or alloys (for example bronzes and brasses) may be sued as the metals in particular embodiments.

As used herein, “metal derivatives” encompass compounds derived from metals, especially oxides, fluorides, chlorides and sulphides.

Such particles may be, in exemplary embodiments, aluminum particles, such as those sold under the names STARBRITE 1200 EAC® by SILBERLINE, and METALURE® by ECKART.

Exemplary embodiments may include metallic powders of copper or of alloy mixtures such as references 2844 sold by RADIUM BRONZE; metal pigments such as aluminum or bronze, such as those sold under the names ROTOSAFE 700 from ECKART; the silica-sheathed aluminum particles sold under the name VISIONAIRE BRIGHT SILVER from ECKART; and the metal alloy particles such as silica-sheathed bronze (copper and zinc alloy) powders sold under the name VISIONAIRE BRIGHT NATURAL gold from ECKART.

The particles of embodiments may be particles including a glass substrate, such as those sold by NIPPON SHEET GLASS under the names MICROGLASS METASHINE.

The goniochromatic colouring agent may be chosen in embodiments from, for example, multilayer interference structures and liquid-crystal colouring agents.

In compositions according to embodiments, symmetrical multilayer interference structures may be used, including, for example, the following structures: Al/SiO₂Al/SiO₂/Al, pigments having this structure being sold by the company DUPONT DE NEMOURS; Cr/MgF₂/Al/MgF₂/Cr, pigments having this structure being sold under the name CHROMAFLAIR by FLEX; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name XIRONA® by MERCK (Darmstadt). By way of example, pigments may be, in embodiments, the pigments of silica/titanium oxide/tin oxide structure sold under the name XIRONA MAGIC by MERCK, the pigments of silica/brown iron oxide structure sold under the name XIRONA INDIAN SUMMER by MERCK; the pigments of silica/titanium oxide/mica/tin oxide structure sold under the name XIRONA CARIBBEAN BLUE by MERCK, and the INFINITE COLORS pigments from the company SHISEIDO. Depending on the thickness and the nature of the various layers, different effects are obtained. Thus, with the Fe₂O₃/SiO₂Al/SiO₂/Fe₂O₃ structure, the color changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

Examples of pigments with a polymeric multilayer structure that may be used in embodiments include those sold by 3M under the name COLOR GLITTER.

Examples of liquid-crystal goniochromatic particles that may be used in embodiments include those sold by CHENIX and the products sold under the name HELICONE® HC by WACKER.

Polyols

According to embodiments, the cosmetic compositions may also include at least one polyol or polyhydric alcohol.

As used herein, the terms “polyhydric alcohol” or “polyol”, encompass any organic molecule containing at least two free hydroxyl groups.

Polyhydric alcohols that may be used in cosmetic compositions according to embodiments include those having, in particular, 2 to 20 carbon atoms, such as 2 to 10 carbon atoms or 2 to 6 carbon atoms.

In exemplary embodiments, the polyol may be chosen, for example, from glycerol; propylene glycol; butylene glycol; pentylene glycol; hexylene glycol; dipropylene glycol; diethylene glycol; sorbitol; hydroxypropylsorbitol, 1,2,6-hexanetriol; glycol ethers, including those glycol ethers having 3 to 16 carbon atoms such as the (C₁-C₄)alkyl ethers of mono-, di- or tripropylene glycol and the (C₁-C₄)alkyl ethers of mono-, di- or triethylene glycol; and mixtures thereof.

Fillers

In exemplary embodiments, the cosmetic compositions may also include at least one filler, of organic or mineral nature, which may allow improved stability with regard to exudation to be imparted.

As used herein, the term “filler” encompasses colorless or white, solid particles of any form, which are in an insoluble and dispersed form in the medium of the composition. Mineral or organic in nature, fillers may give body or rigidity to the composition, and/or softness, and/or a matte effect and/or uniformity to the composition.

The fillers used in the compositions of embodiments may be of lamellar, globular, spherical or fibrous form or in any other form intermediate between these defined forms.

The fillers according to embodiments may or may not be surface-coated; for example, fillers may be surface-treated with silicones, amino acids, fluoro derivatives or any other substance that promotes the dispersion and compatibility of the filler in the composition.

As used herein, the terms “mineral fillers” and “inorganic fillers” are interchangeable.

The mineral fillers that may be used in embodiments may be chosen from talc, mica, silica, trimethyl siloxysilicate, kaolin, bentone, precipitated calcium carbonate, magnesium carbonate, magnesium hydrocarbonate, hydroxyapatite, boron nitride, hollow silica microspheres (Silica Beads from Maprecos), glass or ceramic microcapsules, silica-based fillers, for instance AEROSIL 200 and AEROSIL 300; SUNSPHERE L-31 and SUNSPHERE H-31 sold by Asahi Glass; CHEMICELEN sold by Asahi Chemical; and composites of silica and of titanium dioxide, for instance the TSG series sold by NIPPON SHEET GLASS, and mixtures thereof.

The organic fillers that may be used in embodiments may be chosen from polyamide powder (NYLON® ORGASOL from Atochem); poly-b-alanine powder and polyethylene powder; polytetrafluoroethylene (TEFLON®) powders; lauroyllysine; starch; powders of tetrafluoroethylene polymers; hollow polymer microspheres such as EXPANCEL (NOBEL INDUSTRIE); precipitated calcium carbonate; magnesium carbonate; magnesium hydrocarbonate; metal soaps derived from organic carboxylic acids containing from 8 to 22 carbon atoms including those containing from 12 to 18 carbon atoms, for example zinc stearate, magnesium stearate or lithium stearate, zinc laurate or magnesium myristate, and POLYPORE® L 200 (Chemdal Corporation); silicone resin microbeads (for example TOSPEARL® from Toshiba); and polyurethane powders, such as powders of crosslinked polyurethane including a copolymer that includes trimethylol hexyllactone. In particular embodiments, the organic filler may be a hexamethylene diisocyanate/trimethylol hexyllactone polymer. Such particles are commercially available, for example, under the name PLASTIC POWDER D-400® or PLASTIC POWDER D-800® from the company TOSHIKI, and mixtures thereof.

The fillers of embodiments may be present in a proportion of from 0.001% to 35% or of from 0.5% to 15% of the total weight of the composition.

The filler of embodiments may be, for example, a filler with a mean particle size of less than 100 μm, such as between 1 and 50 μm or between 4 and 20 μm.

According to particular embodiments, the composition includes at least one filler present in a proportion of from 0.01% to 60% of the total weight of the composition, such as from 0.5% to 20% or from 1% to 10% by weight relative to the total weight of the composition.

Additives

The cosmetic compositions according to exemplary embodiments may also comprise any additive commonly used in the field, such as, for example, additives chosen from gelling agents; surfactants such as those described in FR 2834452, which is incorporated herein in its entirety by reference; antioxidants; essential oils; preservatives; perfumes; neutralizing agents; moisturizers; antiseptics; vitamins such as vitamin B3, vitamin E and derivatives thereof; and UV protectants.

According to particular embodiments, the silicone surfactants of non-crosslinked type are included in the compositions.

According to particular embodiments, the may be devoid of ammonium-type surfactants.

Of course, the person skilled in the art will take care to select the optional additive(s) added to the cosmetic composition according to embodiments in such a way that the intrinsic properties of the composition are not, or not substantially, adversely affected by the addition.

According to exemplary embodiments, the silicone polymer of general formula (I) may be chosen from polyglyceryl-3 polydimethylsiloxyethyl dimethicone, lauryl polyglyceryl-3 polydimethylsiloxyethyl dimethicone, polyglyceryl-3 disiloxane dimethicone, and mixtures thereof.

According to exemplary embodiments, the silicone polymer of general formula (I) may be chosen from the silicone polymers sold by SHIN-ETSU under references KF 6100®, KF 6104®, KF 6105®, and mixtures thereof.

According to exemplary embodiments, the cosmetic composition includes polyglyceryl-3 polydimethylsiloxyethyl dimethicone and, as film former, acrylate/stearyl acrylate/dimethicone methacrylate copolymer, such as that sold under reference KP 561® by SHIN-ETSU.

According to exemplary embodiments, the cosmetic composition includes polyglyceryl-3 polydimethylsiloxyethyl dimethicone and at least one wax, which may be chosen from polyethylene waxes, candelilla wax, hydrogenated cocoglyceride wax, and mixtures thereof.

According to exemplary embodiments, the cosmetic composition includes polyglyceryl-3 polydimethylsiloxyethyl dimethicone and at least one hydrocarbon ester containing less than 40 carbon atoms, which may be chosen from stearyl heptanoate, isononyl isononanoate and isotridecyl isononanoate, and mixtures thereof.

In some exemplary embodiments, individual components may belong at one or more different classes of compounds included in the embodiments. For example, isononyl isononanoate may be both a non-volatile oil and a hydrocarbon ester containing less than 40 carbon atoms.

Accordingly, it is not outside the bounds of customary work of the person skilled in the art to adjust the amount of a compound belonging to more than one different class of product to obtain a desired effect.

The cosmetic composition according to exemplary embodiments may be in the form of a lip makeup product, such as a lipstick, or a lip balm.

The examples of compositions below are given by way of illustration and are not to be construed as in any way limiting.

### EXAMPLE 1

### Lipstick

A lipstick is prepared that includes the components set forth in Table I.

TABLE I

| | Percentages |
|---|---|
| | by mass |
| Hydrogenated polyisobutene (Parleam HV from NOF) | 4 |
| Isononyl isononanoate | 12 |
| 2-Octyldodecanol | 4.5 |
| Diisostearyl malate | 33.9 |
| Triglycerides of lauric/palmitic/cetyl/stearic | 4 |
| (50/20/10/10) acids (Softisan 100 ® from Sasol) | |
| N-lauroyl L-lysine | 1 |
| Acrylates/stearyl acrylate/dimethicone methacrylate | 4 |
| copolymer (KP 561 P ® from Shin Etsu) | |
| Dimethicone 6 cSt (KF 96) | 4 |
| Polyglyceryl-3 polydimethylsiloxyethyl dimethicone | 13 |
| (KF6104 ® from Shin Etsu) | |
| Preservative | qs |
| Polyethylene wax (NW 500) | 6.6 |
| Polyethylene wax (MW 400) | 3.8 |
| Hydrophobic pyrogenic silica surface-treated | 2 |
| with dimethylsilane (Aerosil R 972 ® from Degussa) | |
| Pigments | 7.0 |
| Simethicone (Antifoam C ® from DOW CORNING) | 0.2 |
| Total | 100 |

Procedure

An oily phase is prepared by mixing, with heating to approximately 95° C., isononyl isononanoate, 2-octyldodecanol and diisostearyl malate with polyglyceryl-3 polydimethylsiloxyethyl dimethicone and dimethicone oil. The oily phase thus prepared is stirred at approximately 95° C. and fillers (N-lauroyl-L-lysine and pyrogenic silica) are added to the mixture.

The waxes, the pigments in the form of a pigment paste, the hydrogenated polyisobutene and the simethicone are then added to the mixture.

The mixture thus obtained is subsequently poured into a lipstick mold and left to cool until a solid composition is obtained.

Thereafter, the staying power, comfort and gloss of this composition are measured in accordance with the protocols described above. Also measured, using the above-described methods, are the staying power and gloss of two commercial products:
Control A: JELLY PLUMPY sold by Maybelline, containing phenyltrimethicone as silicone polymer;
Control B: AUBE ROUGE GLACÉ sold by Kao and containing the glycerolated silicone of formula below as silicone polymer

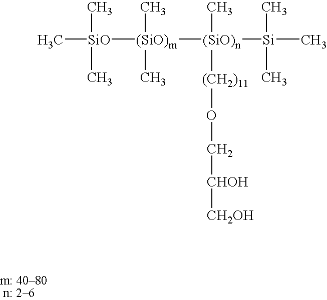

The results obtained are reported in Table II below.

TABLE II

| | Water | Oil | | Comfort | Average |
|---|---|---|---|---|---|
| Formula | resistance | resistance | Transfer | index | gloss |
| Control A | 14.4 | 23.4 | 47.3 | | 42.5 |
| Control B | 7.0 | 8.5 | 42.0 | 85.6 | 48.2 |
| Invention | 5.9 | 7.9 | 32.1 | 99.5 | 48.3 |

In these measurements, the color of the inner surface of the forearm is such that L*=63.9, a*=8.4, b*=13.3, and the color of the paper tissue is such that L*=97.9, a*=0.6 and b*=3.3.

The lipstick of Example 1 has a better staying power and better comfort than the available commercial products tested as Control A and Control B, for an equivalent or equal gloss.

Moreover, the lipstick of Example 1 migrates three to four times less than the Control B.

### EXAMPLES 2 AND 3

### Lipsticks

Lipsticks are prepared including the components set forth in Table III. The percentages are by weight.

TABLE III

| | Example 2 | Example 3 |
|---|---|---|
| Hydrogenated polyisobutene | | 4 |
| (PARLEAM HV from NOF) | | |
| Hydrogenated polyisobutene | 6 | |
| (PARLEAM LITE from NOF) | | |
| Isononyl isononanoate | 8 | 12 |
| 2-Octyldodecanol | 4.5 | 4.5 |
| Diisostearyl malate | 33.9 | 23.9 |
| Polyglyceryl-2 diisostearate | | 5 |
| (COSMOL 42 V from Nishin Oil) | | |
| Polyglyceryl-2 triisostearate | | 4 |
| (COSMOL 43 N from Nishin Oil) | | |
| Sorbitan Sesquioleate | | 1 |
| (COSMOL 82 from Nishin Oil) | | |
| Triglycerides of | 3 | 4 |
| lauric/palmitic/cetyl/stearic | | |
| (50/20/10/10) acids | | |
| (SOFTISAN 100 ® from Sasol) | | |
| N-lauroyl-L-lysine | 1 | 1 |
| Polyvinyl laurate | 3 | |
| Acrylates/stearyl | 2 | 4 |
| acrylate/dimethicone | | |
| methacrylate copolymer | | |
| (KP 561 P ® from Shin Etsu) | | |
| Dimethicone 6 cSt (KF 96) | 5 | 4 |
| Polyglyceryl-3 | 13 | 13 |
| polydimethylsiloxyethyl | | |
| dimethicone | | |
| (KF6104 ® from Shin Etsu) | | |
| Preservative | qs | |
| Polyethylene wax (MW 500) | 5.5 | 6.6 |
| Polyethylene wax (MW 400) | 1.1 | |
| Candelilla wax | 4.8 | |
| Microcrystalline wax | | 3.8 |
| Hydrophobic pyrogenic silica | 2 | 2 |
| surface-treated with | | |
| dimethylsilane | | |
| (AEROSIL R 972 ® from Degussa) | | |
| Pigments | 7.0 | 7 |
| Simethicone | 0.2 | 0.2 |
| (ANTIFOAM C ® from DOW CORNING) | | |
| Total | 100 | 100 |

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

What is claimed is:

1. An anhydrous cosmetic composition comprising, in a physiologically acceptable medium, at least one film former and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (III)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
wherein when R² is a radical of general formula (IIIA):
—C₃H₆—O[CH₂CH(OH)CH₂O]ₙH (IIIA)
in which n is an integer in a range of from 1 to 5, R¹ is not a C₁₂ alkyl radical;
R³ is an organosiloxane group of general formula (IV): 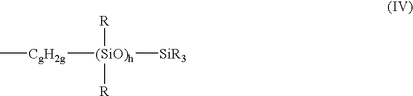
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

2. An anhydrous cosmetic composition comprising, in a physiologically acceptable medium, at least one film former and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
R³ is an organosiloxane group of general formula (IV): 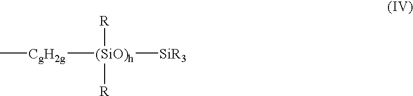
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500; and
said film former is present in a weight ratio, relative to the weight of the silicone polymer of general formula (I), of less than about 5:1.

3. An anhydrous cosmetic comprising, in a physiologically acceptable medium, at least one non-crystalline film former, at least one silicone compound different from the film former, and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
R³ is an organosiloxane group of general formula (IV): 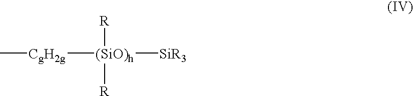
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

4. A cosmetic lip makeup composition comprising, in a physiologically acceptable medium, at least one film former and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
R³ is an organosiloxane group of general formula (IV): 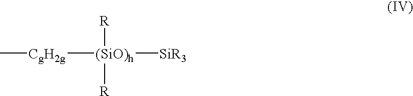
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500; and
wherein said film former is a film former chosen from the group consisting of non-silicone film formers and silicone film formers devoid of acrylic units.

5. A cosmetic composition comprising, in a physiologically acceptable medium, at least one hydrocarbon ester containing less than about 40 carbon atoms, at least one film former and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
R³ is an organosiloxane group of general formula (IV): 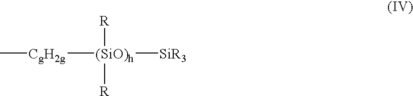
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500; and
wherein said film former is a film former chosen from the group consisting of non-silicone film formers and silicone film formers devoid of acrylic units.

6. The anhydrous cosmetic composition according claim 1, wherein the silicone polymer is a compound of general formula (I) in which R¹ is a C₁ to C₁₀ alkyl radical.

7. The anhydrous cosmetic composition according to claim 1, wherein the silicone polymer is a compound of general formula (I) in which:
a is in a range of from about 1 to about 1.4,
b and c are each independently in a range of from about 0.02 to about 0.04,
R¹ is a C₁ to C₁₀ alkyl radical,
R² is represented by the formula (IIIA):
—C₃H₆O[CH₂CH(OH)CH₂O]ₙH (IIIA)
in which n is in a range of from about 1 to about 5,
R³ is represented by the formula (IVA):
—C₂H₄(CH₃)₂SiO[(CH₃)₂SiO]ₘSi(CH₃)₃ (IVA)
in which m is in a range of from about 3 to about 9.

8. The anhydrous cosmetic composition according to claim 7, wherein the silicone polymer is a compound of general formula (I):
R¹ₐR²_{b}R³_{c}SiO_{(4-a-b-c)/2} (I)
in which:
a is in a range of from about 1 to about 1.4,
b and c are each independently in a range of from about 0.02 to about 0.04, and
R¹ is a methyl radical,
R² is represented by the formula (IIIA) in which n is in a range from about 1 to about 5, and
R³ is represented by the formula (IVA) in which m is in a range from about 3 to about 9.

9. The anhydrous cosmetic composition according to claim 1, wherein said film former chosen from the group consisting of free-radical polymers, polycondensates and polymers of natural origin, polyurethanes, polyesters, polyamides, polyureas and cellulosic polymers.

10. The anhydrous cosmetic composition according to claim 1, wherein said film former is present in a weight ratio, relative to the weight of the silicone polymer of general formula (I), of less than about 5:1.

11. The anhydrous cosmetic composition according to claim 1, wherein said film former is a non-crystalline film former.

12. The anhydrous cosmetic composition according to claim 1, wherein said film former is a non-silicone film former.

13. The anhydrous cosmetic composition according to claim 1, wherein said film former is a silicone film former devoid of acrylic units

14. The anhydrous cosmetic composition according to claim 1, wherein said composition further comprises at least one hydrocarbon ester containing less than about 40 carbon atoms.

15. The anhydrous cosmetic composition according to claim 14, wherein said hydrocarbon ester is a monoester.

16. The anhydrous cosmetic composition according to claim 14, wherein said hydrocarbon ester corresponds to the formula RCOOR′ in which RCOO is a fatty acid residue containing about 2 to about 28 carbon atoms and R′ is a hydrocarbon chain containing from 1 to about 28 carbon atoms.

17. The anhydrous cosmetic composition according to claim 14, wherein said hydrocarbon ester contains less than about 22 carbon atoms.

18. The anhydrous cosmetic composition according to claim 14, wherein said hydrocarbon ester is selected from synthetic esters; hydroxylated esters; esters of neopentanoic acid; esters of isononanoic acid; esters of isopropyl alcohol; cetyl octanoate, tridecyl octanoate, 2-ethylhexyl 4-diheptanoate and palmitate, alkyl benzoate, polyethylene glycol diheptanoate, propylene glycol di-2-ethylhexanoate and mixtures thereof.

19. The anhydrous cosmetic composition according to claim 18, wherein said hydrocarbon ester is selected from purcellin oil, isopropyl myristate, ethyl palmitate, octyl stearate, isostearyl lactate, octyl hydroxystearate, diisopropyl adipate, fatty alcohol heptanoates, octanoates and decanoates, isodecyl neopentanoate, isotridecyl neopentanoate, isostearyl neopentanoate, octyldodecyl neopentanoate, isononyl isononanoate, octyl isononanoate, isodecyl isononanoate, isotridecyl isononanoate, isostearyl isononanoate, isopropyl myristate, isopropyl palmitate, isopropyl stearate or isostearate, cetyl octanoate, tridecyl octanoate, 2-ethylhexyl 4-diheptanoate and palmitate, alkyl benzoate, polyethylene glycol diheptanoate, propylene glycol di-2-ethylhexanoate and mixtures thereof.

20. The anhydrous cosmetic composition according to claim 1, further comprising at least one hydrocarbon oil chosen from the group consisting of hydrogenated polyisobutene, isostearyl heptanoate, isononyl isononanoate, dipentaerythritol tetrahydroxystearate/tetraisostearate, 2-octyldodecanol, isotridecyl isononanoate, diisostearyl malate, and mixtures thereof.

21. The anhydrous cosmetic composition according to claim 1, further comprising at least one silicone compound.

22. The anhydrous cosmetic composition according to claim 21, wherein said silicone compound is chosen from the group consisting of silicone oils, silicone gums, silicone resins, silicone elastomers, and mixtures thereof.

23. The anhydrous cosmetic composition according to claim 22, wherein said silicone polymer is an acrylate/stearyl acrylate/silicone methacrylate copolymer.

24. The anhydrous cosmetic composition according to claim 22, wherein said silicone oil is chosen from the group consisting of:
linear and cyclic silicone oils, and
silicone oils including non-volatile polydimethylsiloxanes (PDMS), polydimethylsiloxanes containing terminal and/or pendent C₂-C₂₄ alkyl or alkoxy groups, phenyl silicones, and
mixtures thereof.

25. The anhydrous cosmetic composition of claim 1, wherein the composition is in cast form.

26. The anhydrous cosmetic composition of claim 1, further comprising at least one colorant.

27. The anhydrous cosmetic composition of claim 1, further comprising at least one compound chosen from the group consisting of waxes, pasty fatty substances, and mixtures thereof.

28. The anhydrous cosmetic composition of claim 1, further comprising at least one filler.

29. The anhydrous cosmetic composition of claim 1, wherein the composition is a skin and/or lip care and/or makeup composition.

30. A method of preparing the anhydrous cosmetic composition according to claim 2, wherein the method comprises:
combining, in a physiologically acceptable medium, at least one film former and at least one silicone polymer
wherein the silicone polymer is of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
R³ is an organosiloxane group of general formula (IV): 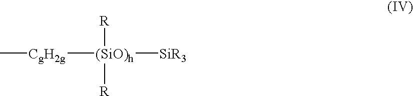
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500; and
said film former is present in a weight ratio, relative to the weight of the silicone polymer of general formula (I), of less than about 5:1.

31. The method according to claim 30, wherein the combining further comprises combining, in the physiologically acceptable medium, the film former, the silicone polymer of general formula (I) and at least one silicone compound.

32. A method for preparing the anhydrous cosmetic composition according to claim 2, wherein the method comprises:
combining, in a physiologically acceptable medium, at least one film former and at least one silicone polymer of general formule (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50, and
combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
wherein when R² is a radical of general formula (IIIA):
—C₃H₆—O[CH₂CH(OH)CH₂O]ₙH (IIIA)
in which n is an integer in a range of from 1 to 5, R¹ is not a C₁₂ alkyl radical;
R³ is an organosiloxane group of general formula (IV): 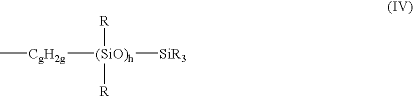
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

33. A method according to claim 32, wherein the combining further comprises combining, in the physiologically acceptable medium, the film former, the silicone polymer of general formula (I) and at least one silicone compound.

34. A method of preparing the anhydrous cosmetic composition according to claim 9, wherein the method comprises:
combining, in a physiologically acceptable medium, at least one film former and at least one silicone polymer of general formula (I).

35. The method according to claim 32, wherein the combining further comprises combining, in the physiologically acceptable medium, the film former, the silicone polymer of general formula (I) and at least one silicone compound.
